SEQUENCE LISTING (659 sequences)

SEQ ID NO: 1 - RNA, 25 nt, Artificial Sequence
  Description of Artificial Sequence: Synthetic oligonucleotide
gccauggacuugaucuugaucaauu

SEQ ID NO: 2 - RNA, 25 nt, Artificial Sequence
  Description of Artificial Sequence: Synthetic oligonucleotide
aauugaucaagaucaaguccauggc

SEQ ID NO: 3 - RNA, 25 nt, Artificial Sequence
  Description of Artificial Sequence: Synthetic oligonucleotide
ccuaccucuuguaucugaugcugaa

SEQ ID NO: 4 - RNA, 25 nt, Artificial Sequence
  Description of Artificial Sequence: Synthetic oligonucleotide
uucagcaucagauacaagagguagg

SEQ ID NO: 5 - RNA, 25 nt, Artificial Sequence
  Description of Artificial Sequence: Synthetic oligonucleotide
ccggcaugaaguaccugauauucua

SEQ ID NO: 6 - RNA, 25 nt, Artificial Sequence
  Description of Artificial Sequence: Synthetic oligonucleotide
uagaauaucagguacuucaugccgg

SEQ ID NO: 7 - RNA, 25 nt, Artificial Sequence
  Description of Artificial Sequence: Synthetic oligonucleotide
aaggacgugugagaaggcuugugaa

SEQ ID NO: 8 - RNA, 25 nt, Artificial Sequence
  Description of Artificial Sequence: Synthetic oligonucleotide
uucacaagccuucucacacguccuu

SEQ ID NO: 9 - RNA, 25 nt, Artificial Sequence
  Description of Artificial Sequence: Synthetic oligonucleotide
cauaacuuugcugucaucaacauca

SEQ ID NO: 10 - RNA, 25 nt, Artificial Sequence
  Description of Artificial Sequence: Synthetic oligonucleotide
ugauguugaugacagcaaaguuaug

SEQ ID NO: 11 - RNA, 25 nt, Artificial Sequence
  Description of Artificial Sequence: Synthetic oligonucleotide
gcaacuugacuucggugcuacuuaa

SEQ ID NO: 12 - RNA, 25 nt, Artificial Sequence
  Description of Artificial Sequence: Synthetic oligonucleotide
uuaaguagcaccgaagucaaguugc

SEQ ID NO: 13 - RNA, 25 nt, Artificial Sequence
  Description of Artificial Sequence: Synthetic oligonucleotide
uggacaauauuggauggcuauucua

SEQ ID NO: 14 - RNA, 25 nt, Artificial Sequence
  Description of Artificial Sequence: Synthetic oligonucleotide
uagaauagccauccaauauugucca

SEQ ID NO: 15 - RNA, 25 nt, Artificial Sequence
  Description of Artificial Sequence: Synthetic oligonucleotide
caggagaacuggaaguucuuuguaa

SEQ ID NO: 16 - RNA, 25 nt, Artificial Sequence
  Description of Artificial Sequence: Synthetic oligonucleotide
uuacaaagaacuuccaguucuccug

SEQ ID NO: 17 - RNA, 25 nt, Artificial Sequence
  Description of Artificial Sequence: Synthetic oligonucleotide
caucaaucucuuaggagcaugugaa

SEQ ID NO: 18 - RNA, 25 nt, Artificial Sequence
  Description of Artificial Sequence: Synthetic oligonucleotide
uucacaugcuccuaagagauugaug

SEQ ID NO: 19 - RNA, 25 nt, Artificial Sequence
  Description of Artificial Sequence: Synthetic oligonucleotide
gaagccuuaugagaggccaucauuu

SEQ ID NO: 20 - RNA, 25 nt, Artificial Sequence
  Description of Artificial Sequence: Synthetic oligonucleotide
aaaugauggccucucauaaggcuuc

SEQ ID NO: 21 - RNA, 25 nt, Artificial Sequence
  Description of Artificial Sequence: Synthetic oligonucleotide
ccaggauccgcuggaaguuacucaa

SEQ ID NO: 22 - RNA, 25 nt, Artificial Sequence
  Description of Artificial Sequence: Synthetic oligonucleotide
uugaguaacuuccagcggauccugg

SEQ ID NO: 23 - RNA, 25 nt, Artificial Sequence
  Description of Artificial Sequence: Synthetic oligonucleotide
cgaggagaggcaaucaggauacgaa

SEQ ID NO: 24 - RNA, 25 nt, Artificial Sequence
  Description of Artificial Sequence: Synthetic oligonucleotide
uucguauccugauugccucuccucg

SEQ ID NO: 25 - RNA, 25 nt, Artificial Sequence
  Description of Artificial Sequence: Synthetic oligonucleotide
gaugcgucaacaagcuuccuuccua

SEQ ID NO: 26 - RNA, 25 nt, Artificial Sequence
  Description of Artificial Sequence: Synthetic oligonucleotide
uaggaaggaagcuuguugacgcauc

SEQ ID NO: 27 - RNA, 25 nt, Artificial Sequence
  Description of Artificial Sequence: Synthetic oligonucleotide
agcuuccuuccuaccagcuacuuua

SEQ ID NO: 28 - RNA, 25 nt, Artificial Sequence
  Description of Artificial Sequence: Synthetic oligonucleotide
uaaaguagcugguaggaaggaagcu

SEQ ID NO: 29 - RNA, 25 nt, Artificial Sequence
  Description of Artificial Sequence: Synthetic oligonucleotide
gacaagggagauaacgugaacauau

SEQ ID NO: 30 - RNA, 25 nt, Artificial Sequence
  Description of Artificial Sequence: Synthetic oligonucleotide
auauguucacguuaucucccuuguc

SEQ ID NO: 31 - RNA, 25 nt, Artificial Sequence
  Description of Artificial Sequence: Synthetic oligonucleotide
caggcugauaguccggagaugugaa

SEQ ID NO: 32 - RNA, 25 nt, Artificial Sequence
  Description of Artificial Sequence: Synthetic oligonucleotide
uucacaucuccggacuaucagccug

SEQ ID NO: 33 - RNA, 24 nt, Artificial Sequence
  Description of Artificial Sequence: Synthetic oligonucleotide
caaccaucucuguacugcuguaug

SEQ ID NO: 34 - RNA, 24 nt, Artificial Sequence
  Description of Artificial Sequence: Synthetic oligonucleotide
cauacagcaguacagagaugguug

SEQ ID NO: 35 - RNA, 25 nt, Artificial Sequence
  Description of Artificial Sequence: Synthetic oligonucleotide
caucucuguacugcuuguaugaaca

SEQ ID NO: 36 - RNA, 25 nt, Artificial Sequence
  Description of Artificial Sequence: Synthetic oligonucleotide
uguucauacaagcaguacagagaug

SEQ ID NO: 37 - RNA, 25 nt, Artificial Sequence
  Description of Artificial Sequence: Synthetic oligonucleotide
gcacacguuuggcagaacuuguaaa

SEQ ID NO: 38 - RNA, 25 nt, Artificial Sequence
  Description of Artificial Sequence: Synthetic oligonucleotide
uuuacaaguucugccaaacgugugc

SEQ ID NO: 39 - RNA, 25 nt, Artificial Sequence
  Description of Artificial Sequence: Synthetic oligonucleotide
aguggacaagagggaugcaagucuu

SEQ ID NO: 40 - RNA, 25 nt, Artificial Sequence
  Description of Artificial Sequence: Synthetic oligonucleotide
aagacuugcaucccucuuguccacu

SEQ ID NO: 41 - RNA, 25 nt, Artificial Sequence
  Description of Artificial Sequence: Synthetic oligonucleotide
aagagggaugcaagucuuauguguu

SEQ ID NO: 42 - RNA, 25 nt, Artificial Sequence
  Description of Artificial Sequence: Synthetic oligonucleotide
aacacauaagacuugcaucccucuu

SEQ ID NO: 43 - RNA, 25 nt, Artificial Sequence
  Description of Artificial Sequence: Synthetic oligonucleotide
gcaaugaagcaugccacccugguuu

SEQ ID NO: 44 - RNA, 25 nt, Artificial Sequence
  Description of Artificial Sequence: Synthetic oligonucleotide
aaaccaggguggcaugcuucauugc

SEQ ID NO: 45 - RNA, 25 nt, Artificial Sequence
  Description of Artificial Sequence: Synthetic oligonucleotide
caaagauaguggauuugccagauca

SEQ ID NO: 46 - RNA, 25 nt, Artificial Sequence
  Description of Artificial Sequence: Synthetic oligonucleotide
ugaucuggcaaauccacuaucuuug

SEQ ID NO: 47 - RNA, 25 nt, Artificial Sequence
  Description of Artificial Sequence: Synthetic oligonucleotide
gauaguggauuugccagaucauaua

SEQ ID NO: 48 - RNA, 25 nt, Artificial Sequence
  Description of Artificial Sequence: Synthetic oligonucleotide
uauaugaucuggcaaauccacuauc

SEQ ID NO: 49 - RNA, 25 nt, Artificial Sequence
  Description of Artificial Sequence: Synthetic oligonucleotide
ggacauaacuuugcugucaucaaca

SEQ ID NO: 50 - RNA, 25 nt, Artificial Sequence
  Description of Artificial Sequence: Synthetic oligonucleotide
uguugaugacagcaaaguuaugucc

SEQ ID NO: 51 - RNA, 25 nt, Artificial Sequence
  Description of Artificial Sequence: Synthetic oligonucleotide
cguuaaucacuaugaggcuuggcaa

SEQ ID NO: 52 - RNA, 25 nt, Artificial Sequence
  Description of Artificial Sequence: Synthetic oligonucleotide
uugccaagccucauagugauuaacg

SEQ ID NO: 53 - RNA, 25 nt, Artificial Sequence
  Description of Artificial Sequence: Synthetic oligonucleotide
acuaugaggcuuggcaacauauuca

SEQ ID NO: 54 - RNA, 25 nt, Artificial Sequence
  Description of Artificial Sequence: Synthetic oligonucleotide
ugaauauguugccaagccucauagu

SEQ ID NO: 55 - RNA, 25 nt, Artificial Sequence
  Description of Artificial Sequence: Synthetic oligonucleotide
ccaagaggucuaaaucuccugccua

SEQ ID NO: 56 - RNA, 25 nt, Artificial Sequence
  Description of Artificial Sequence: Synthetic oligonucleotide
uaggcaggagauuuagaccucuugg

SEQ ID NO: 57 - RNA, 25 nt, Artificial Sequence
  Description of Artificial Sequence: Synthetic oligonucleotide
caagaggucuaaaucuccugccuaa

SEQ ID NO: 58 - RNA, 25 nt, Artificial Sequence
  Description of Artificial Sequence: Synthetic oligonucleotide
uuaggcaggagauuuagaccucuug

SEQ ID NO: 59 - RNA, 25 nt, Artificial Sequence
  Description of Artificial Sequence: Synthetic oligonucleotide
agcagaauauuaaaguuccaggcaa

SEQ ID NO: 60 - RNA, 25 nt, Artificial Sequence
  Description of Artificial Sequence: Synthetic oligonucleotide
uugccuggaacuuuaauauucugcu

SEQ ID NO: 61 - RNA, 25 nt, Artificial Sequence
  Description of Artificial Sequence: Synthetic oligonucleotide
caggcaacuugacuucggugcuacu

SEQ ID NO: 62 - RNA, 25 nt, Artificial Sequence
  Description of Artificial Sequence: Synthetic oligonucleotide
aguagcaccgaagucaaguugccug

SEQ ID NO: 63 - RNA, 25 nt, Artificial Sequence
  Description of Artificial Sequence: Synthetic oligonucleotide
gaagaucucacugcuuggacccuua

SEQ ID NO: 64 - RNA, 25 nt, Artificial Sequence
  Description of Artificial Sequence: Synthetic oligonucleotide
uaaggguccaagcagugagaucuuc

SEQ ID NO: 65 - RNA, 25 nt, Artificial Sequence
  Description of Artificial Sequence: Synthetic oligonucleotide
cccuuagugacauucuuccuccuca

SEQ ID NO: 66 - RNA, 25 nt, Artificial Sequence
  Description of Artificial Sequence: Synthetic oligonucleotide
ugaggaggaagaaugucacuaaggg

SEQ ID NO: 67 - RNA, 25 nt, Artificial Sequence
  Description of Artificial Sequence: Synthetic oligonucleotide
ccuuagugacauucuuccuccucaa

SEQ ID NO: 68 - RNA, 25 nt, Artificial Sequence
  Description of Artificial Sequence: Synthetic oligonucleotide
uugaggaggaagaaugucacuaagg

SEQ ID NO: 69 - RNA, 25 nt, Artificial Sequence
  Description of Artificial Sequence: Synthetic oligonucleotide
aggagaacuggaaguucuuuguaaa

SEQ ID NO: 70 - RNA, 25 nt, Artificial Sequence
  Description of Artificial Sequence: Synthetic oligonucleotide
uuuacaaagaacuuccaguucuccu

SEQ ID NO: 71 - RNA, 25 nt, Artificial Sequence
  Description of Artificial Sequence: Synthetic oligonucleotide
gaggcuacuuguaccuggccauuga

SEQ ID NO: 72 - RNA, 25 nt, Artificial Sequence
  Description of Artificial Sequence: Synthetic oligonucleotide
ucaauggccagguacaaguagccuc

SEQ ID NO: 73 - RNA, 25 nt, Artificial Sequence
  Description of Artificial Sequence: Synthetic oligonucleotide
gaaaccuucuggacuuccuucgcaa

SEQ ID NO: 74 - RNA, 25 nt, Artificial Sequence
  Description of Artificial Sequence: Synthetic oligonucleotide
uugcgaaggaaguccagaagguuuc

SEQ ID NO: 75 - RNA, 25 nt, Artificial Sequence
  Description of Artificial Sequence: Synthetic oligonucleotide
ucgagucacugaauuacagugugua

SEQ ID NO: 76 - RNA, 25 nt, Artificial Sequence
  Description of Artificial Sequence: Synthetic oligonucleotide
uacacacuguaauucagugacucga

SEQ ID NO: 77 - RNA, 25 nt, Artificial Sequence
  Description of Artificial Sequence: Synthetic oligonucleotide
gcgggaugacuugugcagaacucua

SEQ ID NO: 78 - RNA, 25 nt, Artificial Sequence
  Description of Artificial Sequence: Synthetic oligonucleotide
uagaguucugcacaagucaucccgc

SEQ ID NO: 79 - RNA, 25 nt, Artificial Sequence
  Description of Artificial Sequence: Synthetic oligonucleotide
cccugaacugugaugaugaggugua

SEQ ID NO: 80 - RNA, 25 nt, Artificial Sequence
  Description of Artificial Sequence: Synthetic oligonucleotide
uacaccucaucaucacaguucaggg

SEQ ID NO: 81 - RNA, 25 nt, Artificial Sequence
  Description of Artificial Sequence: Synthetic oligonucleotide
gaggagcgaaagaccuacgugaaua

SEQ ID NO: 82 - RNA, 25 nt, Artificial Sequence
  Description of Artificial Sequence: Synthetic oligonucleotide
uauucacguaggucuuucgcuccuc

SEQ ID NO: 83 - RNA, 25 nt, Artificial Sequence
  Description of Artificial Sequence: Synthetic oligonucleotide
ggacuugaucuugaucaauucccua

SEQ ID NO: 84 - RNA, 25 nt, Artificial Sequence
  Description of Artificial Sequence: Synthetic oligonucleotide
uagggaauugaucaagaucaagucc

SEQ ID NO: 85 - RNA, 25 nt, Artificial Sequence
  Description of Artificial Sequence: Synthetic oligonucleotide
ugaucuugaucaauucccuaccucu

SEQ ID NO: 86 - RNA, 25 nt, Artificial Sequence
  Description of Artificial Sequence: Synthetic oligonucleotide
agagguagggaauugaucaagauca

SEQ ID NO: 87 - RNA, 25 nt, Artificial Sequence
  Description of Artificial Sequence: Synthetic oligonucleotide
caauucccuaccucuuguaucugau

SEQ ID NO: 88 - RNA, 25 nt, Artificial Sequence
  Description of Artificial Sequence: Synthetic oligonucleotide
aucagauacaagagguagggaauug

SEQ ID NO: 89 - RNA, 25 nt, Artificial Sequence
  Description of Artificial Sequence: Synthetic oligonucleotide
ggauccgcuggaaguuacucaagau

SEQ ID NO: 90 - RNA, 25 nt, Artificial Sequence
  Description of Artificial Sequence: Synthetic oligonucleotide
aucuugaguaacuuccagcggaucc

SEQ ID NO: 91 - RNA, 25 nt, Artificial Sequence
  Description of Artificial Sequence: Synthetic oligonucleotide
aggcaaucaggauacgaaccaugaa

SEQ ID NO: 92 - RNA, 25 nt, Artificial Sequence
  Description of Artificial Sequence: Synthetic oligonucleotide
uucaugguucguauccugauugccu

SEQ ID NO: 93 - RNA, 25 nt, Artificial Sequence
  Description of Artificial Sequence: Synthetic oligonucleotide
ggagauaacgugaacauaucuuuca

SEQ ID NO: 94 - RNA, 25 nt, Artificial Sequence
  Description of Artificial Sequence: Synthetic oligonucleotide
ugaaagauauguucacguuaucucc

SEQ ID NO: 95 - RNA, 25 nt, Artificial Sequence
  Description of Artificial Sequence: Synthetic oligonucleotide
gccagguauauaggaggaaaccucu

SEQ ID NO: 96 - RNA, 25 nt, Artificial Sequence
  Description of Artificial Sequence: Synthetic oligonucleotide
agagguuuccuccuauauaccuggc

SEQ ID NO: 97 - RNA, 25 nt, Artificial Sequence
  Description of Artificial Sequence: Synthetic oligonucleotide
ccagguauauaggaggaaaccucuu

SEQ ID NO: 98 - RNA, 25 nt, Artificial Sequence
  Description of Artificial Sequence: Synthetic oligonucleotide
aagagguuuccuccuauauaccugg

SEQ ID NO: 99 - RNA, 25 nt, Artificial Sequence
  Description of Artificial Sequence: Synthetic oligonucleotide
ugucugccaugaagauacuggagaa

SEQ ID NO: 100 - RNA, 25 nt, Artificial Sequence
  Description of Artificial Sequence: Synthetic oligonucleotide
uucuccaguaucuucauggcagaca

SEQ ID NO: 101 - RNA, 25 nt, Artificial Sequence
  Description of Artificial Sequence: Synthetic oligonucleotide
cacguuuggcagaacuuguaaagaa

SEQ ID NO: 102 - RNA, 25 nt, Artificial Sequence
  Description of Artificial Sequence: Synthetic oligonucleotide
uucuuuacaaguucugccaaacgug

SEQ ID NO: 103 - RNA, 25 nt, Artificial Sequence
  Description of Artificial Sequence: Synthetic oligonucleotide
uggacaagagggaugcaagucuuau

SEQ ID NO: 104 - RNA, 25 nt, Artificial Sequence
  Description of Artificial Sequence: Synthetic oligonucleotide
auaagacuugcaucccucuugucca

SEQ ID NO: 105 - RNA, 25 nt, Artificial Sequence
  Description of Artificial Sequence: Synthetic oligonucleotide
gagaugugugaucgcuuccaaggau

SEQ ID NO: 106 - RNA, 25 nt, Artificial Sequence
  Description of Artificial Sequence: Synthetic oligonucleotide
auccuuggaagcgaucacacaucuc

SEQ ID NO: 107 - RNA, 25 nt, Artificial Sequence
  Description of Artificial Sequence: Synthetic oligonucleotide
gaucgcuuccaaggaugucucugcu

SEQ ID NO: 108 - RNA, 25 nt, Artificial Sequence
  Description of Artificial Sequence: Synthetic oligonucleotide
agcagagacauccuuggaagcgauc

SEQ ID NO: 109 - RNA, 25 nt, Artificial Sequence
  Description of Artificial Sequence: Synthetic oligonucleotide
caaacgugauugacacuggacauaa

SEQ ID NO: 110 - RNA, 25 nt, Artificial Sequence
  Description of Artificial Sequence: Synthetic oligonucleotide
uuauguccagugucaaucacguuug

SEQ ID NO: 111 - RNA, 25 nt, Artificial Sequence
  Description of Artificial Sequence: Synthetic oligonucleotide
acacuggacauaacuuugcugucau

SEQ ID NO: 112 - RNA, 25 nt, Artificial Sequence
  Description of Artificial Sequence: Synthetic oligonucleotide
augacagcaaaguuauguccagugu

SEQ ID NO: 113 - RNA, 25 nt, Artificial Sequence
  Description of Artificial Sequence: Synthetic oligonucleotide
ucaucaacaucagcucugagccuua

SEQ ID NO: 114 - RNA, 25 nt, Artificial Sequence
  Description of Artificial Sequence: Synthetic oligonucleotide
uaaggcucagagcugauguugauga

SEQ ID NO: 115 - RNA, 25 nt, Artificial Sequence
  Description of Artificial Sequence: Synthetic oligonucleotide
ucaacaucagcucugagccuuacuu

SEQ ID NO: 116 - RNA, 25 nt, Artificial Sequence
  Description of Artificial Sequence: Synthetic oligonucleotide
aaguaaggcucagagcugauguuga

SEQ ID NO: 117 - RNA, 25 nt, Artificial Sequence
  Description of Artificial Sequence: Synthetic oligonucleotide
caacaucagcucugagccuuacuuu

SEQ ID NO: 118 - RNA, 25 nt, Artificial Sequence
  Description of Artificial Sequence: Synthetic oligonucleotide
aaaguaaggcucagagcugauguug

SEQ ID NO: 119 - RNA, 25 nt, Artificial Sequence
  Description of Artificial Sequence: Synthetic oligonucleotide
agaagcuucuauacaaacccguuaa

SEQ ID NO: 120 - RNA, 25 nt, Artificial Sequence
  Description of Artificial Sequence: Synthetic oligonucleotide
uuaacggguuuguauagaagcuucu

SEQ ID NO: 121 - RNA, 25 nt, Artificial Sequence
  Description of Artificial Sequence: Synthetic oligonucleotide
gaagcuucuauacaaacccguuaau

SEQ ID NO: 122 - RNA, 25 nt, Artificial Sequence
  Description of Artificial Sequence: Synthetic oligonucleotide
auuaacggguuuguauagaagcuuc

SEQ ID NO: 123 - RNA, 25 nt, Artificial Sequence
  Description of Artificial Sequence: Synthetic oligonucleotide
ccguuaaucacuaugaggcuuggca

SEQ ID NO: 124 - RNA, 25 nt, Artificial Sequence
  Description of Artificial Sequence: Synthetic oligonucleotide
ugccaagccucauagugauuaacgg

SEQ ID NO: 125 - RNA, 25 nt, Artificial Sequence
  Description of Artificial Sequence: Synthetic oligonucleotide
gaccacucuaaauuugaccuggcaa

SEQ ID NO: 126 - RNA, 25 nt, Artificial Sequence
  Description of Artificial Sequence: Synthetic oligonucleotide
uugccaggucaaauuuagagugguc

SEQ ID NO: 127 - RNA, 25 nt, Artificial Sequence
  Description of Artificial Sequence: Synthetic oligonucleotide
gacuucggugcuacuuaacaacuua

SEQ ID NO: 128 - RNA, 25 nt, Artificial Sequence
  Description of Artificial Sequence: Synthetic oligonucleotide
uaaguuguuaaguagcaccgaaguc

SEQ ID NO: 129 - RNA, 25 nt, Artificial Sequence
  Description of Artificial Sequence: Synthetic oligonucleotide
acacacuccucggcugugauuucuu

SEQ ID NO: 130 - RNA, 25 nt, Artificial Sequence
  Description of Artificial Sequence: Synthetic oligonucleotide
aagaaaucacagccgaggagugugu

SEQ ID NO: 131 - RNA, 25 nt, Artificial Sequence
  Description of Artificial Sequence: Synthetic oligonucleotide
cacguugaugugaagauaaagaaug

SEQ ID NO: 132 - RNA, 25 nt, Artificial Sequence
  Description of Artificial Sequence: Synthetic oligonucleotide
cauucuuuaucuucacaucaacgug

SEQ ID NO: 133 - RNA, 25 nt, Artificial Sequence
  Description of Artificial Sequence: Synthetic oligonucleotide
gaagauaaagaaugccaccaucauu

SEQ ID NO: 134 - RNA, 25 nt, Artificial Sequence
  Description of Artificial Sequence: Synthetic oligonucleotide
aaugaugguggcauucuuuaucuuc

SEQ ID NO: 135 - RNA, 25 nt, Artificial Sequence
  Description of Artificial Sequence: Synthetic oligonucleotide
cagagaacaacauagggucaagcaa

SEQ ID NO: 136 - RNA, 25 nt, Artificial Sequence
  Description of Artificial Sequence: Synthetic oligonucleotide
uugcuugacccuauguuguucucug

SEQ ID NO: 137 - RNA, 25 nt, Artificial Sequence
  Description of Artificial Sequence: Synthetic oligonucleotide
gaagaugcugcuuauagccauccuu

SEQ ID NO: 138 - RNA, 25 nt, Artificial Sequence
  Description of Artificial Sequence: Synthetic oligonucleotide
aaggauggcuauaagcagcaucuuc

SEQ ID NO: 139 - RNA, 25 nt, Artificial Sequence
  Description of Artificial Sequence: Synthetic oligonucleotide
uagccauccuuggcucugcuggaau

SEQ ID NO: 140 - RNA, 25 nt, Artificial Sequence
  Description of Artificial Sequence: Synthetic oligonucleotide
auuccagcagagccaaggauggcua

SEQ ID NO: 141 - RNA, 25 nt, Artificial Sequence
  Description of Artificial Sequence: Synthetic oligonucleotide
ucaacucagggacucuggcccuaaa

SEQ ID NO: 142 - RNA, 25 nt, Artificial Sequence
  Description of Artificial Sequence: Synthetic oligonucleotide
uuuagggccagagucccugaguuga

SEQ ID NO: 143 - RNA, 25 nt, Artificial Sequence
  Description of Artificial Sequence: Synthetic oligonucleotide
acucuggcccuaaacaggaagguca

SEQ ID NO: 144 - RNA, 25 nt, Artificial Sequence
  Description of Artificial Sequence: Synthetic oligonucleotide
ugaccuuccuguuuagggccagagu

SEQ ID NO: 145 - RNA, 25 nt, Artificial Sequence
  Description of Artificial Sequence: Synthetic oligonucleotide
acuuugcaggagaacuggaaguucu

SEQ ID NO: 146 - RNA, 25 nt, Artificial Sequence
  Description of Artificial Sequence: Synthetic oligonucleotide
agaacuuccaguucuccugcaaagu

SEQ ID NO: 147 - RNA, 25 nt, Artificial Sequence
  Description of Artificial Sequence: Synthetic oligonucleotide
gcaggagaacuggaaguucuuugua

SEQ ID NO: 148 - RNA, 25 nt, Artificial Sequence
  Description of Artificial Sequence: Synthetic oligonucleotide
uacaaagaacuuccaguucuccugc

SEQ ID NO: 149 - RNA, 25 nt, Artificial Sequence
  Description of Artificial Sequence: Synthetic oligonucleotide
uggaaguucuuuguaaacuuggaca

SEQ ID NO: 150 - RNA, 25 nt, Artificial Sequence
  Description of Artificial Sequence: Synthetic oligonucleotide
uguccaaguuuacaaagaacuucca

SEQ ID NO: 151 - RNA, 25 nt, Artificial Sequence
  Description of Artificial Sequence: Synthetic oligonucleotide
ggaaaccuucuggacuuccuucgca

SEQ ID NO: 152 - RNA, 25 nt, Artificial Sequence
  Description of Artificial Sequence: Synthetic oligonucleotide
ugcgaaggaaguccagaagguuucc

SEQ ID NO: 153 - RNA, 25 nt, Artificial Sequence
  Description of Artificial Sequence: Synthetic oligonucleotide
gacccagcauuugccauugccaaua

SEQ ID NO: 154 - RNA, 25 nt, Artificial Sequence
  Description of Artificial Sequence: Synthetic oligonucleotide
uauuggcaauggcaaaugcuggguc

SEQ ID NO: 155 - RNA, 25 nt, Artificial Sequence
  Description of Artificial Sequence: Synthetic oligonucleotide
ccgaggucaagagguguacgugaaa

SEQ ID NO: 156 - RNA, 25 nt, Artificial Sequence
  Description of Artificial Sequence: Synthetic oligonucleotide
uuucacguacaccucuugaccucgg

SEQ ID NO: 157 - RNA, 25 nt, Artificial Sequence
  Description of Artificial Sequence: Synthetic oligonucleotide
ugguguguuacuaugggagauuguu

SEQ ID NO: 158 - RNA, 25 nt, Artificial Sequence
  Description of Artificial Sequence: Synthetic oligonucleotide
aacaaucucccauaguaacacacca

SEQ ID NO: 159 - RNA, 25 nt, Artificial Sequence
  Description of Artificial Sequence: Synthetic oligonucleotide
gguguguuacuaugggagauuguua

SEQ ID NO: 160 - RNA, 25 nt, Artificial Sequence
  Description of Artificial Sequence: Synthetic oligonucleotide
uaacaaucucccauaguaacacacc

SEQ ID NO: 161 - RNA, 25 nt, Artificial Sequence
  Description of Artificial Sequence: Synthetic oligonucleotide
aagaccuacgugaauaccacgcuuu

SEQ ID NO: 162 - RNA, 25 nt, Artificial Sequence
  Description of Artificial Sequence: Synthetic oligonucleotide
aaagcgugguauucacguaggucuu

SEQ ID NO: 163 - RNA, 25 nt, Artificial Sequence
  Description of Artificial Sequence: Synthetic oligonucleotide
gaccuacgugaauaccacgcuuuau

SEQ ID NO: 164 - RNA, 25 nt, Artificial Sequence
  Description of Artificial Sequence: Synthetic oligonucleotide
auaaagcgugguauucacguagguc

SEQ ID NO: 165 - RNA, 25 nt, Artificial Sequence
  Description of Artificial Sequence: Synthetic oligonucleotide
ccacgcuuuaugagaaguuuacuua

SEQ ID NO: 166 - RNA, 25 nt, Artificial Sequence
  Description of Artificial Sequence: Synthetic oligonucleotide
uaaguaaacuucucauaaagcgugg

SEQ ID NO: 167 - RNA, 25 nt, Artificial Sequence
  Description of Artificial Sequence: Synthetic oligonucleotide
caggcugauuguucggagaugugaa

SEQ ID NO: 168 - RNA, 25 nt, Artificial Sequence
  Description of Artificial Sequence: Synthetic oligonucleotide
uucacaucuccgaacaaucagccug

SEQ ID NO: 169 - RNA, 25 nt, Artificial Sequence
  Description of Artificial Sequence: Synthetic oligonucleotide
cguccuuguacuacuugcaagaaca

SEQ ID NO: 170 - RNA, 25 nt, Artificial Sequence
  Description of Artificial Sequence: Synthetic oligonucleotide
uguucuugcaaguaguacaaggacg

SEQ ID NO: 171 - RNA, 25 nt, Artificial Sequence
  Description of Artificial Sequence: Synthetic oligonucleotide
gaaagcuugugagccgcacacauuu

SEQ ID NO: 172 - RNA, 25 nt, Artificial Sequence
  Description of Artificial Sequence: Synthetic oligonucleotide
aaaugugugcggcucacaagcuuuc

SEQ ID NO: 173 - RNA, 25 nt, Artificial Sequence
  Description of Artificial Sequence: Synthetic oligonucleotide
cagaaggaugcaagucuuauguguu

SEQ ID NO: 174 - RNA, 25 nt, Artificial Sequence
  Description of Artificial Sequence: Synthetic oligonucleotide
aacacauaagacuugcauccuucug

SEQ ID NO: 175 - RNA, 25 nt, Artificial Sequence
  Description of Artificial Sequence: Synthetic oligonucleotide
caggccaaggaugacuccacagaua

SEQ ID NO: 176 - RNA, 25 nt, Artificial Sequence
  Description of Artificial Sequence: Synthetic oligonucleotide
uaucuguggagucauccuuggccug

SEQ ID NO: 177 - RNA, 25 nt, Artificial Sequence
  Description of Artificial Sequence: Synthetic oligonucleotide
cacagauagaggauuugccagauca

SEQ ID NO: 178 - RNA, 25 nt, Artificial Sequence
  Description of Artificial Sequence: Synthetic oligonucleotide
ugaucuggcaaauccucuaucugug

SEQ ID NO: 179 - RNA, 25 nt, Artificial Sequence
  Description of Artificial Sequence: Synthetic oligonucleotide
uggguggccacuaccuacuagugaa

SEQ ID NO: 180 - RNA, 25 nt, Artificial Sequence
  Description of Artificial Sequence: Synthetic oligonucleotide
uucacuaguagguaguggccaccca

SEQ ID NO: 181 - RNA, 25 nt, Artificial Sequence
  Description of Artificial Sequence: Synthetic oligonucleotide
caagaggucucagucuccugccaaa

SEQ ID NO: 182 - RNA, 25 nt, Artificial Sequence
  Description of Artificial Sequence: Synthetic oligonucleotide
uuuggcaggagacugagaccucuug

SEQ ID NO: 183 - RNA, 25 nt, Artificial Sequence
  Description of Artificial Sequence: Synthetic oligonucleotide
gcgaucccugcaaacaacaagugau

SEQ ID NO: 184 - RNA, 25 nt, Artificial Sequence
  Description of Artificial Sequence: Synthetic oligonucleotide
aucacuuguuguuugcagggaucgc

SEQ ID NO: 185 - RNA, 25 nt, Artificial Sequence
  Description of Artificial Sequence: Synthetic oligonucleotide
agcagaacaucaaagugccugggaa

SEQ ID NO: 186 - RNA, 25 nt, Artificial Sequence
  Description of Artificial Sequence: Synthetic oligonucleotide
uucccaggcacuuugauguucugcu

SEQ ID NO: 187 - RNA, 25 nt, Artificial Sequence
  Description of Artificial Sequence: Synthetic oligonucleotide
aaggugccauggaccugaucuugau

SEQ ID NO: 188 - RNA, 25 nt, Artificial Sequence
  Description of Artificial Sequence: Synthetic oligonucleotide
aucaagaucagguccauggcaccuu

SEQ ID NO: 189 - RNA, 25 nt, Artificial Sequence
  Description of Artificial Sequence: Synthetic oligonucleotide
gccauggaccugaucuugaucaauu

SEQ ID NO: 190 - RNA, 25 nt, Artificial Sequence
  Description of Artificial Sequence: Synthetic oligonucleotide
aauugaucaagaucagguccauggc

SEQ ID NO: 191 - RNA, 25 nt, Artificial Sequence
  Description of Artificial Sequence: Synthetic oligonucleotide
ccuaccucuugugucugaugccgaa

SEQ ID NO: 192 - RNA, 25 nt, Artificial Sequence
  Description of Artificial Sequence: Synthetic oligonucleotide
uucggcaucagacacaagagguagg

SEQ ID NO: 193 - RNA, 25 nt, Artificial Sequence
  Description of Artificial Sequence: Synthetic oligonucleotide
caucaccauaggaagggacuuugaa

SEQ ID NO: 194 - RNA, 25 nt, Artificial Sequence
  Description of Artificial Sequence: Synthetic oligonucleotide
uucaaagucccuuccuauggugaug

SEQ ID NO: 195 - RNA, 25 nt, Artificial Sequence
  Description of Artificial Sequence: Synthetic oligonucleotide
ccaagauccacuggagguuacucaa

SEQ ID NO: 196 - RNA, 25 nt, Artificial Sequence
  Description of Artificial Sequence: Synthetic oligonucleotide
uugaguaaccuccaguggaucuugg

SEQ ID NO: 197 - RNA, 25 nt, Artificial Sequence
  Description of Artificial Sequence: Synthetic oligonucleotide
ggccaguaagauuaauggugcuuau

SEQ ID NO: 198 - RNA, 25 nt, Artificial Sequence
  Description of Artificial Sequence: Synthetic oligonucleotide
auaagcaccauuaaucuuacuggcc

SEQ ID NO: 199 - RNA, 25 nt, Artificial Sequence
  Description of Artificial Sequence: Synthetic oligonucleotide
gaugcgucaacaagcguccuuccua

SEQ ID NO: 200 - RNA, 25 nt, Artificial Sequence
  Description of Artificial Sequence: Synthetic oligonucleotide
uaggaaggacgcuuguugacgcauc

SEQ ID NO: 201 - RNA, 25 nt, Artificial Sequence
  Description of Artificial Sequence: Synthetic oligonucleotide
agcguccuuccuaccugcuacuuua

SEQ ID NO: 202 - RNA, 25 nt, Artificial Sequence
  Description of Artificial Sequence: Synthetic oligonucleotide
uaaaguagcagguaggaaggacgcu

SEQ ID NO: 203 - RNA, 25 nt, Artificial Sequence
  Description of Artificial Sequence: Synthetic oligonucleotide
ccagguacauaggaggaaaccuguu

SEQ ID NO: 204 - RNA, 25 nt, Artificial Sequence
  Description of Artificial Sequence: Synthetic oligonucleotide
aacagguuuccuccuauguaccugg

SEQ ID NO: 205 - RNA, 25 nt, Artificial Sequence
  Description of Artificial Sequence: Synthetic oligonucleotide
cgacuguagccguccuuguacuacu

SEQ ID NO: 206 - RNA, 25 nt, Artificial Sequence
  Description of Artificial Sequence: Synthetic oligonucleotide
aguaguacaaggacggcuacagucg

SEQ ID NO: 207 - RNA, 25 nt, Artificial Sequence
  Description of Artificial Sequence: Synthetic oligonucleotide
gagaacaugugagaaagcuugugag

SEQ ID NO: 208 - RNA, 25 nt, Artificial Sequence
  Description of Artificial Sequence: Synthetic oligonucleotide
cucacaagcuuucucacauguucuc

SEQ ID NO: 209 - RNA, 25 nt, Artificial Sequence
  Description of Artificial Sequence: Synthetic oligonucleotide
gaaagcuugugagccgcacacauuu

SEQ ID NO: 210 - RNA, 25 nt, Artificial Sequence
  Description of Artificial Sequence: Synthetic oligonucleotide
aaaugugugcggcucacaagcuuuc

SEQ ID NO: 211 - RNA, 25 nt, Artificial Sequence
  Description of Artificial Sequence: Synthetic oligonucleotide
cgcacacauuuggcaggaccuguaa

SEQ ID NO: 212 - RNA, 25 nt, Artificial Sequence
  Description of Artificial Sequence: Synthetic oligonucleotide
uuacagguccugccaaaugugugcg

SEQ ID NO: 213 - RNA, 25 nt, Artificial Sequence
  Description of Artificial Sequence: Synthetic oligonucleotide
gcacacauuuggcaggaccuguaaa

SEQ ID NO: 214 - RNA, 25 nt, Artificial Sequence
  Description of Artificial Sequence: Synthetic oligonucleotide
uuuacagguccugccaaaugugugc

SEQ ID NO: 215 - RNA, 25 nt, Artificial Sequence
  Description of Artificial Sequence: Synthetic oligonucleotide
aguggaccagaaggaugcaagucuu

SEQ ID NO: 216 - RNA, 25 nt, Artificial Sequence
  Description of Artificial Sequence: Synthetic oligonucleotide
aagacuugcauccuucugguccacu

SEQ ID NO: 217 - RNA, 25 nt, Artificial Sequence
  Description of Artificial Sequence: Synthetic oligonucleotide
gacuguaagcucaggugccacugua

SEQ ID NO: 218 - RNA, 25 nt, Artificial Sequence
  Description of Artificial Sequence: Synthetic oligonucleotide
uacaguggcaccugagcuuacaguc

SEQ ID NO: 219 - RNA, 25 nt, Artificial Sequence
  Description of Artificial Sequence: Synthetic oligonucleotide
caaccgagucuuaccuccugacuca

SEQ ID NO: 220 - RNA, 25 nt, Artificial Sequence
  Description of Artificial Sequence: Synthetic oligonucleotide
ugagucaggagguaagacuagguug

SEQ ID NO: 221 - RNA, 25 nt, Artificial Sequence
  Description of Artificial Sequence: Synthetic oligonucleotide
ccugucaaucaggccuggaaauaca

SEQ ID NO: 222 - RNA, 25 nt, Artificial Sequence
  Description of Artificial Sequence: Synthetic oligonucleotide
uguauuuccaggccugauugacagg

SEQ ID NO: 223 - RNA, 25 nt, Artificial Sequence
  Description of Artificial Sequence: Synthetic oligonucleotide
caaucaggccuggaaauacauugaa

SEQ ID NO: 224 - RNA, 25 nt, Artificial Sequence
  Description of Artificial Sequence: Synthetic oligonucleotide
uucaauguauuuccaggccugauug

SEQ ID NO: 225 - RNA, 25 nt, Artificial Sequence
  Description of Artificial Sequence: Synthetic oligonucleotide
cacagcuaugguuucuuggacaaua

SEQ ID NO: 226 - RNA, 25 nt, Artificial Sequence
  Description of Artificial Sequence: Synthetic oligonucleotide
uauuguccaagaaaccauagcugug

SEQ ID NO: 227 - RNA, 25 nt, Artificial Sequence
  Description of Artificial Sequence: Synthetic oligonucleotide
gaccagcacauugaugugaagauca

SEQ ID NO: 228 - RNA, 25 nt, Artificial Sequence
  Description of Artificial Sequence: Synthetic oligonucleotide
ugaucuucacaucaaugugcugguc

SEQ ID NO: 229 - RNA, 25 nt, Artificial Sequence
  Description of Artificial Sequence: Synthetic oligonucleotide
cacauugaugugaagaucaagaaug

SEQ ID NO: 230 - RNA, 25 nt, Artificial Sequence
  Description of Artificial Sequence: Synthetic oligonucleotide
cauucuugaucuucacaucaaugug

SEQ ID NO: 231 - RNA, 25 nt, Artificial Sequence
  Description of Artificial Sequence: Synthetic oligonucleotide
ccuagagccagagacuacauaccau

SEQ ID NO: 232 - RNA, 25 nt, Artificial Sequence
  Description of Artificial Sequence: Synthetic oligonucleotide
augguauguagucucuggcucuagg

SEQ ID NO: 233 - RNA, 25 nt, Artificial Sequence
  Description of Artificial Sequence: Synthetic oligonucleotide
aaacaauccggaucccacaauuuau

SEQ ID NO: 234 - RNA, 25 nt, Artificial Sequence
  Description of Artificial Sequence: Synthetic oligonucleotide
auaaauugugggauccggauuguuu

SEQ ID NO: 235 - RNA, 25 nt, Artificial Sequence
  Description of Artificial Sequence: Synthetic oligonucleotide
ggaaugacaucaaguuucaagacgu

SEQ ID NO: 236 - RNA, 25 nt, Artificial Sequence
  Description of Artificial Sequence: Synthetic oligonucleotide
acgucuugaaacuugaugucauucc

SEQ ID NO: 237 - RNA, 25 nt, Artificial Sequence
  Description of Artificial Sequence: Synthetic oligonucleotide
ccgccaucaagaggaugaaagagua

SEQ ID NO: 238 - RNA, 25 nt, Artificial Sequence
  Description of Artificial Sequence: Synthetic oligonucleotide
uacucuuucauccucuugauggcgg

SEQ ID NO: 239 - RNA, 25 nt, Artificial Sequence
  Description of Artificial Sequence: Synthetic oligonucleotide
gaggaugaaagaguaugccuccaaa

SEQ ID NO: 240 - RNA, 25 nt, Artificial Sequence
  Description of Artificial Sequence: Synthetic oligonucleotide
uuuggaggcauacucuuucauaauc

SEQ ID NO: 241 - RNA, 25 nt, Artificial Sequence
  Description of Artificial Sequence: Synthetic oligonucleotide
gcaggagaacuggagguucuuugua

SEQ ID NO: 242 - RNA, 25 nt, Artificial Sequence
  Description of Artificial Sequence: Synthetic oligonucleotide
uacaaagaaccuccaguucuccugc

SEQ ID NO: 243 - RNA, 25 nt, Artificial Sequence
  Description of Artificial Sequence: Synthetic oligonucleotide
caggagaacuggagguucuuuguaa

SEQ ID NO: 244 - RNA, 25 nt, Artificial Sequence
  Description of Artificial Sequence: Synthetic oligonucleotide
uuacaaagaaccuccaguucuccug

SEQ ID NO: 245 - RNA, 25 nt, Artificial Sequence
  Description of Artificial Sequence: Synthetic oligonucleotide
aggagaacuggagguucuuuguaaa

SEQ ID NO: 246 - RNA, 25 nt, Artificial Sequence
  Description of Artificial Sequence: Synthetic oligonucleotide
uuuacaaagaaccuccaguucuccu

SEQ ID NO: 247 - RNA, 25 nt, Artificial Sequence
  Description of Artificial Sequence: Synthetic oligonucleotide
caucaaucucuugggagcaugugaa

SEQ ID NO: 248 - RNA, 25 nt, Artificial Sequence
  Description of Artificial Sequence: Synthetic oligonucleotide
uucacaugcucccaagagauugaug

SEQ ID NO: 249 - RNA, 25 nt, Artificial Sequence
  Description of Artificial Sequence: Synthetic oligonucleotide
caccgaggcuauuuguaccuagcua

SEQ ID NO: 250 - RNA, 25 nt, Artificial Sequence
  Description of Artificial Sequence: Synthetic oligonucleotide
uagcuagguacaaauagccucggug

SEQ ID NO: 251 - RNA, 25 nt, Artificial Sequence
  Description of Artificial Sequence: Synthetic oligonucleotide
ccgaggcuauuuguaccuagcuauu

SEQ ID NO: 252 - RNA, 25 nt, Artificial Sequence
  Description of Artificial Sequence: Synthetic oligonucleotide
aauagcuagguacaaauagccucgg

SEQ ID NO: 253 - RNA, 25 nt, Artificial Sequence
  Description of Artificial Sequence: Synthetic oligonucleotide
gaggcuauuuguaccuagcuauuga

SEQ ID NO: 254 - RNA, 25 nt, Artificial Sequence
  Description of Artificial Sequence: Synthetic oligonucleotide
ucaauagcuagguacaaauagccuc

SEQ ID NO: 255 - RNA, 25 nt, Artificial Sequence
  Description of Artificial Sequence: Synthetic oligonucleotide
gauugucacgaggucaagaagugua

SEQ ID NO: 256 - RNA, 25 nt, Artificial Sequence
  Description of Artificial Sequence: Synthetic oligonucleotide
uacacuucuugaccucgugacaauc

SEQ ID NO: 257 - RNA, 25 nt, Artificial Sequence
  Description of Artificial Sequence: Synthetic oligonucleotide
cacgaggucaagaaguguaugugaa

SEQ ID NO: 258 - RNA, 25 nt, Artificial Sequence
  Description of Artificial Sequence: Synthetic oligonucleotide
uucacauacacuucuugaccucgug

SEQ ID NO: 259 - RNA, 25 nt, Artificial Sequence
  Description of Artificial Sequence: Synthetic oligonucleotide
ccagugcguuggauggcaaucgaau

SEQ ID NO: 260 - RNA, 25 nt, Artificial Sequence
  Description of Artificial Sequence: Synthetic oligonucleotide
auucgauugccauccaacgcacugg

SEQ ID NO: 261 - RNA, 25 nt, Artificial Sequence
  Description of Artificial Sequence: Synthetic oligonucleotide
cacacuguaugagaaguuuaccuau

SEQ ID NO: 262 - RNA, 25 nt, Artificial Sequence
  Description of Artificial Sequence: Synthetic oligonucleotide
auagguaaacuucucauacagugug

SEQ ID NO: 263 - RNA, 25 nt, Artificial Sequence
  Description of Artificial Sequence: Synthetic oligonucleotide
ugaucuugaucaauucccuaccucu

SEQ ID NO: 264 - RNA, 25 nt, Artificial Sequence
  Description of Artificial Sequence: Synthetic oligonucleotide
agagguagggaauugaucaagauca

SEQ ID NO: 265 - RNA, 25 nt, Artificial Sequence
  Description of Artificial Sequence: Synthetic oligonucleotide
ccaucaccauaggaagggacuuuga

SEQ ID NO: 266 - RNA, 25 nt, Artificial Sequence
  Description of Artificial Sequence: Synthetic oligonucleotide
ucaaagucccuuccuauggugaugg

SEQ ID NO: 267 - RNA, 25 nt, Artificial Sequence
  Description of Artificial Sequence: Synthetic oligonucleotide
caucaccauaggaagggacuuugaa

SEQ ID NO: 268 - RNA, 25 nt, Artificial Sequence
  Description of Artificial Sequence: Synthetic oligonucleotide
uucaaagucccuuccuauggugaug

SEQ ID NO: 269 - RNA, 25 nt, Artificial Sequence
  Description of Artificial Sequence: Synthetic oligonucleotide
cccugaacugugaugaugaggugua

SEQ ID NO: 270 - RNA, 25 nt, Artificial Sequence
  Description of Artificial Sequence: Synthetic oligonucleotide
uacaccucaucaucacaguucaggg

SEQ ID NO: 271 - RNA, 25 nt, Artificial Sequence
  Description of Artificial Sequence: Synthetic oligonucleotide
cauuuagagacugugcagauguaua

SEQ ID NO: 272 - RNA, 25 nt, Artificial Sequence
  Description of Artificial Sequence: Synthetic oligonucleotide
uauacaucugcacagucucuaaaug

SEQ ID NO: 273 - RNA, 25 nt, Artificial Sequence
  Description of Artificial Sequence: Synthetic oligonucleotide
acaacaucgugaagauggaagucua

SEQ ID NO: 274 - RNA, 25 nt, Artificial Sequence
  Description of Artificial Sequence: Synthetic oligonucleotide
uagacuuccaucuucacgauguugu

SEQ ID NO: 275 - RNA, 25 nt, Artificial Sequence
  Description of Artificial Sequence: Synthetic oligonucleotide
gauuuccaaagaggcuggaaggaau

SEQ ID NO: 276 - RNA, 25 nt, Artificial Sequence
  Description of Artificial Sequence: Synthetic oligonucleotide
auuccuuccagccucuuuggaaauc

SEQ ID NO: 277 - RNA, 25 nt, Artificial Sequence
  Description of Artificial Sequence: Synthetic oligonucleotide
aagaauugaguuaauggacugggaa

SEQ ID NO: 278 - RNA, 25 nt, Artificial Sequence
  Description of Artificial Sequence: Synthetic oligonucleotide
uucccaguccauuaacucaauucuu

SEQ ID NO: 279 - RNA, 25 nt, Artificial Sequence
  Description of Artificial Sequence: Synthetic oligonucleotide
cagccugaucuuacacggugcugau

SEQ ID NO: 280 - RNA, 25 nt, Artificial Sequence
  Description of Artificial Sequence: Synthetic oligonucleotide
aucagcaccguguaagaucaggcug

SEQ ID NO: 281 - RNA, 25 nt, Artificial Sequence
  Description of Artificial Sequence: Synthetic oligonucleotide
cccuccaaucuaaauggaauguucu

SEQ ID NO: 282 - RNA, 25 nt, Artificial Sequence
  Description of Artificial Sequence: Synthetic oligonucleotide
agaacauuccauuuagauuggaggg

SEQ ID NO: 283 - RNA, 25 nt, Artificial Sequence
  Description of Artificial Sequence: Synthetic oligonucleotide
ccuccaaucuaaauggaauguucua

SEQ ID NO: 284 - RNA, 25 nt, Artificial Sequence
  Description of Artificial Sequence: Synthetic oligonucleotide
uagaacauuccauuuagauuggagg

SEQ ID NO: 285 - RNA, 25 nt, Artificial Sequence
  Description of Artificial Sequence: Synthetic oligonucleotide
caguuacuccuuacguuccacaacu

SEQ ID NO: 286 - RNA, 25 nt, Artificial Sequence
  Description of Artificial Sequence: Synthetic oligonucleotide
aguuguggaacguaaggaguaacug

SEQ ID NO: 287 - RNA, 25 nt, Artificial Sequence
  Description of Artificial Sequence: Synthetic oligonucleotide
ccacaacuaugaugauucgaccuuu

SEQ ID NO: 288 - RNA, 25 nt, Artificial Sequence
  Description of Artificial Sequence: Synthetic oligonucleotide
aaaggucgaaucaucauaguugugg

SEQ ID NO: 289 - RNA, 25 nt, Artificial Sequence
  Description of Artificial Sequence: Synthetic oligonucleotide
cacaacuaugaugauucgaccuuua

SEQ ID NO: 290 - RNA, 25 nt, Artificial Sequence
  Description of Artificial Sequence: Synthetic oligonucleotide
uaaaggucgaaucaucauaguugug

SEQ ID NO: 291 - RNA, 25 nt, Artificial Sequence
  Description of Artificial Sequence: Synthetic oligonucleotide
ggagaagauauaaccggauucaaca

SEQ ID NO: 292 - RNA, 25 nt, Artificial Sequence
  Description of Artificial Sequence: Synthetic oligonucleotide
uguugaauccgguuauaucuucucc

SEQ ID NO: 293 - RNA, 25 nt, Artificial Sequence
  Description of Artificial Sequence: Synthetic oligonucleotide
caacaugggcaaugugccuacacuu

SEQ ID NO: 294 - RNA, 25 nt, Artificial Sequence
  Description of Artificial Sequence: Synthetic oligonucleotide
aaguguaggcacauugcccauguug

SEQ ID NO: 295 - RNA, 25 nt, Artificial Sequence
  Description of Artificial Sequence: Synthetic oligonucleotide
caugggcaaugugccuacacuuuca

SEQ ID NO: 296 - RNA, 25 nt, Artificial Sequence
  Description of Artificial Sequence: Synthetic oligonucleotide
ugaaaguguaggcacauugcccaug

SEQ ID NO: 297 - RNA, 25 nt, Artificial Sequence
  Description of Artificial Sequence: Synthetic oligonucleotide
ccuacacuuucauucuuccagaaca

SEQ ID NO: 298 - RNA, 25 nt, Artificial Sequence
  Description of Artificial Sequence: Synthetic oligonucleotide
uguucuggaagaaugaaaguguagg

SEQ ID NO: 299 - RNA, 25 nt, Artificial Sequence
  Description of Artificial Sequence: Synthetic oligonucleotide
cagcagaaugcaguucagaaccaca

SEQ ID NO: 300 - RNA, 25 nt, Artificial Sequence
  Description of Artificial Sequence: Synthetic oligonucleotide
ugugguucugaacugcauucugcug

SEQ ID NO: 301 - RNA, 25 nt, Artificial Sequence
  Description of Artificial Sequence: Synthetic oligonucleotide
ccuucaaggcuugguuacucgucaa

SEQ ID NO: 302 - RNA, 25 nt, Artificial Sequence
  Description of Artificial Sequence: Synthetic oligonucleotide
uugacgaguaaccaagccuugaagg

SEQ ID NO: 303 - RNA, 25 nt, Artificial Sequence
  Description of Artificial Sequence: Synthetic oligonucleotide
caguaugacagauuccacauaggaa

SEQ ID NO: 304 - RNA, 25 nt, Artificial Sequence
  Description of Artificial Sequence: Synthetic oligonucleotide
uuccuauguggaaucugucauacug

SEQ ID NO: 305 - RNA, 25 nt, Artificial Sequence
  Description of Artificial Sequence: Synthetic oligonucleotide
caggaggauggugguuugaugcuug

SEQ ID NO: 306 - RNA, 25 nt, Artificial Sequence
  Description of Artificial Sequence: Synthetic oligonucleotide
caagcaucaaaccaccauccuccug

SEQ ID NO: 307 - RNA, 25 nt, Artificial Sequence
  Description of Artificial Sequence: Synthetic oligonucleotide
gauauaaccggauucaacaugggca

SEQ ID NO: 308 - RNA, 25 nt, Artificial Sequence
  Description of Artificial Sequence: Synthetic oligonucleotide
ugcccauguugaauccgguuauauc

SEQ ID NO: 309 - RNA, 25 nt, Artificial Sequence
  Description of Artificial Sequence: Synthetic oligonucleotide
ucaacaugggcaaugugccuacacu

SEQ ID NO: 310 - RNA, 25 nt, Artificial Sequence
  Description of Artificial Sequence: Synthetic oligonucleotide
aguguaggcacauugcccauguuga

SEQ ID NO: 311 - RNA, 25 nt, Artificial Sequence
  Description of Artificial Sequence: Synthetic oligonucleotide
cagauguugagacccagguacuaaa

SEQ ID NO: 312 - RNA, 25 nt, Artificial Sequence
  Description of Artificial Sequence: Synthetic oligonucleotide
uuuaguaccugggucucaacaucug

SEQ ID NO: 313 - RNA, 25 nt, Artificial Sequence
  Description of Artificial Sequence: Synthetic oligonucleotide
gacagauuccacauaggaaaugaaa

SEQ ID NO: 314 - RNA, 25 nt, Artificial Sequence
  Description of Artificial Sequence: Synthetic oligonucleotide
uuucauuuccuauguggaaucuguc

SEQ ID NO: 315 - RNA, 25 nt, Artificial Sequence
  Description of Artificial Sequence: Synthetic oligonucleotide
ugaaugggauaaaguggcacuacuu

SEQ ID NO: 316 - RNA, 25 nt, Artificial Sequence
  Description of Artificial Sequence: Synthetic oligonucleotide
aaguagugccacuuuaucccauuca

SEQ ID NO: 317 - RNA, 25 nt, Artificial Sequence
  Description of Artificial Sequence: Synthetic oligonucleotide
ggcacuacuucaaagggcccaguua

SEQ ID NO: 318 - RNA, 25 nt, Artificial Sequence
  Description of Artificial Sequence: Synthetic oligonucleotide
uaacugggcccuuugaaguagugcc

SEQ ID NO: 319 - RNA, 25 nt, Artificial Sequence
  Description of Artificial Sequence: Synthetic oligonucleotide
cgugagaguacgacagaccaguaca

SEQ ID NO: 320 - RNA, 25 nt, Artificial Sequence
  Description of Artificial Sequence: Synthetic oligonucleotide
uguacuggucugucguacucucacg

SEQ ID NO: 321 - RNA, 25 nt, Artificial Sequence
  Description of Artificial Sequence: Synthetic oligonucleotide
gagaguacgacagaccaguacaaca

SEQ ID NO: 322 - RNA, 25 nt, Artificial Sequence
  Description of Artificial Sequence: Synthetic oligonucleotide
uguuguacuggucugucguacucuc

SEQ ID NO: 323 - RNA, 25 nt, Artificial Sequence
  Description of Artificial Sequence: Synthetic oligonucleotide
cagaccaguacaacacaaacgcucu

SEQ ID NO: 324 - RNA, 25 nt, Artificial Sequence
  Description of Artificial Sequence: Synthetic oligonucleotide
agagcguuuguguuguacuggucug

SEQ ID NO: 325 - RNA, 25 nt, Artificial Sequence
  Description of Artificial Sequence: Synthetic oligonucleotide
uccacacguggaaccggauuucucu

SEQ ID NO: 326 - RNA, 25 nt, Artificial Sequence
  Description of Artificial Sequence: Synthetic oligonucleotide
agagaaauccgguuccacgugugga

SEQ ID NO: 327 - RNA, 25 nt, Artificial Sequence
  Description of Artificial Sequence: Synthetic oligonucleotide
ccacacguggaaccggauuucucuu

SEQ ID NO: 328 - RNA, 25 nt, Artificial Sequence
  Description of Artificial Sequence: Synthetic oligonucleotide
aagagaaauccgguuccacgugugg

SEQ ID NO: 329 - RNA, 25 nt, Artificial Sequence
  Description of Artificial Sequence: Synthetic oligonucleotide
caacaucuggaacaugugauggaaa

SEQ ID NO: 330 - RNA, 25 nt, Artificial Sequence
  Description of Artificial Sequence: Synthetic oligonucleotide
uuuccaucacauguuccagauguug

SEQ ID NO: 331 - RNA, 25 nt, Artificial Sequence
  Description of Artificial Sequence: Synthetic oligonucleotide
ggcccagauacagcagaaugcaguu

SEQ ID NO: 332 - RNA, 25 nt, Artificial Sequence
  Description of Artificial Sequence: Synthetic oligonucleotide
aacugcauucugcuguaucugggcc

SEQ ID NO: 333 - RNA, 25 nt, Artificial Sequence
  Description of Artificial Sequence: Synthetic oligonucleotide
ccagauacagcagaaugcaguucag

SEQ ID NO: 334 - RNA, 25 nt, Artificial Sequence
  Description of Artificial Sequence: Synthetic oligonucleotide
cugaacugcauucugcuguaucugg

SEQ ID NO: 335 - RNA, 25 nt, Artificial Sequence
  Description of Artificial Sequence: Synthetic oligonucleotide
agauacagcagaaugcaguucagaa

SEQ ID NO: 336 - RNA, 25 nt, Artificial Sequence
  Description of Artificial Sequence: Synthetic oligonucleotide
uucugaacugcauucugcuguaucu

SEQ ID NO: 337 - RNA, 25 nt, Artificial Sequence
  Description of Artificial Sequence: Synthetic oligonucleotide
gaaugcaguucagaaccacacggcu

SEQ ID NO: 338 - RNA, 25 nt, Artificial Sequence
  Description of Artificial Sequence: Synthetic oligonucleotide
agccgugugguucugaacugcauuc

SEQ ID NO: 339 - RNA, 25 nt, Artificial Sequence
  Description of Artificial Sequence: Synthetic oligonucleotide
gguacuaaaucaaacuucucgacuu

SEQ ID NO: 340 - RNA, 25 nt, Artificial Sequence
  Description of Artificial Sequence: Synthetic oligonucleotide
aagucgagaaguuugauuuaguacc

SEQ ID NO: 341 - RNA, 25 nt, Artificial Sequence
  Description of Artificial Sequence: Synthetic oligonucleotide
gacuugagauacagcugcuggagaa

SEQ ID NO: 342 - RNA, 25 nt, Artificial Sequence
  Description of Artificial Sequence: Synthetic oligonucleotide
uucuccagcagcuguaucucaaguc

SEQ ID NO: 343 - RNA, 25 nt, Artificial Sequence
  Description of Artificial Sequence: Synthetic oligonucleotide
gaaccuucaaggcuugguuacucgu

SEQ ID NO: 344 - RNA, 25 nt, Artificial Sequence
  Description of Artificial Sequence: Synthetic oligonucleotide
acgaguaaccaagccuugaagguuc

SEQ ID NO: 345 - RNA, 25 nt, Artificial Sequence
  Description of Artificial Sequence: Synthetic oligonucleotide
accuucaaggcuugguuacucguca

SEQ ID NO: 346 - RNA, 25 nt, Artificial Sequence
  Description of Artificial Sequence: Synthetic oligonucleotide
ugacgaguaaccaagccuugaaggu

SEQ ID NO: 347 - RNA, 25 nt, Artificial Sequence
  Description of Artificial Sequence: Synthetic oligonucleotide
caaggcuugguuacucgucaaacau

SEQ ID NO: 348 - RNA, 25 nt, Artificial Sequence
  Description of Artificial Sequence: Synthetic oligonucleotide
auguuugacgaguaaccaagccuug

SEQ ID NO: 349 - RNA, 25 nt, Artificial Sequence
  Description of Artificial Sequence: Synthetic oligonucleotide
aggcuugguuacucgucaaacauau

SEQ ID NO: 350 - RNA, 25 nt, Artificial Sequence
  Description of Artificial Sequence: Synthetic oligonucleotide
auauguuugacgaguaaccaagccu

SEQ ID NO: 351 - RNA, 25 nt, Artificial Sequence
  Description of Artificial Sequence: Synthetic oligonucleotide
gcuugguuacucgucaaacauauau

SEQ ID NO: 352 - RNA, 25 nt, Artificial Sequence
  Description of Artificial Sequence: Synthetic oligonucleotide
auauauguuugacgaguaaccaagc

SEQ ID NO: 353 - RNA, 25 nt, Artificial Sequence
  Description of Artificial Sequence: Synthetic oligonucleotide
uggacacaguccacaaccuugucaa

SEQ ID NO: 354 - RNA, 25 nt, Artificial Sequence
  Description of Artificial Sequence: Synthetic oligonucleotide
uugacaagguuguggacugugucca

SEQ ID NO: 355 - RNA, 25 nt, Artificial Sequence
  Description of Artificial Sequence: Synthetic oligonucleotide
cacaguccacaaccuugucaaucuu

SEQ ID NO: 356 - RNA, 25 nt, Artificial Sequence
  Description of Artificial Sequence: Synthetic oligonucleotide
aagauugacaagguuguggacugug

SEQ ID NO: 357 - RNA, 25 nt, Artificial Sequence
  Description of Artificial Sequence: Synthetic oligonucleotide
caguccacaaccuugucaaucuuug

SEQ ID NO: 358 - RNA, 25 nt, Artificial Sequence
  Description of Artificial Sequence: Synthetic oligonucleotide
caaagauugacaagguuguggacug

SEQ ID NO: 359 - RNA, 25 nt, Artificial Sequence
  Description of Artificial Sequence: Synthetic oligonucleotide
ccacaaccuugucaaucuuugcacu

SEQ ID NO: 360 - RNA, 25 nt, Artificial Sequence
  Description of Artificial Sequence: Synthetic oligonucleotide
agugcaaagauugacaagguugugg

SEQ ID NO: 361 - RNA, 25 nt, Artificial Sequence
  Description of Artificial Sequence: Synthetic oligonucleotide
gaagagaaaccauuuagagacugug

SEQ ID NO: 362 - RNA, 25 nt, Artificial Sequence
  Description of Artificial Sequence: Synthetic oligonucleotide
cacagucucuaaaugguuucucuuc

SEQ ID NO: 363 - RNA, 25 nt, Artificial Sequence
  Description of Artificial Sequence: Synthetic oligonucleotide
accauuuagagacugugcagaugua

SEQ ID NO: 364 - RNA, 25 nt, Artificial Sequence
  Description of Artificial Sequence: Synthetic oligonucleotide
uacaucugcacagucucuaaauggu

SEQ ID NO: 365 - RNA, 25 nt, Artificial Sequence
  Description of Artificial Sequence: Synthetic oligonucleotide
uagagacugugcagauguauaucaa

SEQ ID NO: 366 - RNA, 25 nt, Artificial Sequence
  Description of Artificial Sequence: Synthetic oligonucleotide
uugauauacaucugcacagucucua

SEQ ID NO: 367 - RNA, 25 nt, Artificial Sequence
  Description of Artificial Sequence: Synthetic oligonucleotide
gauggaagucuagauuuccaaagag

SEQ ID NO: 368 - RNA, 25 nt, Artificial Sequence
  Description of Artificial Sequence: Synthetic oligonucleotide
cucuuuggaaaucuagacuuccauc

SEQ ID NO: 369 - RNA, 25 nt, Artificial Sequence
  Description of Artificial Sequence: Synthetic oligonucleotide
ucagaggcaguacaugcuaagaauu

SEQ ID NO: 370 - RNA, 25 nt, Artificial Sequence
  Description of Artificial Sequence: Synthetic oligonucleotide
aauucuuagcauguacugccucuga

SEQ ID NO: 371 - RNA, 25 nt, Artificial Sequence
  Description of Artificial Sequence: Synthetic oligonucleotide
cgagccuauucacaguaugacagau

SEQ ID NO: 372 - RNA, 25 nt, Artificial Sequence
  Description of Artificial Sequence: Synthetic oligonucleotide
aucugucauacugugaauaggcucg

SEQ ID NO: 373 - RNA, 25 nt, Artificial Sequence
  Description of Artificial Sequence: Synthetic oligonucleotide
ugacagauuccacauaggaaaugaa

SEQ ID NO: 374 - RNA, 25 nt, Artificial Sequence
  Description of Artificial Sequence: Synthetic oligonucleotide
uucauuuccuauguggaaucuguca

SEQ ID NO: 375 - RNA, 25 nt, Artificial Sequence
  Description of Artificial Sequence: Synthetic oligonucleotide
acacggugcugauuucagcacuaaa

SEQ ID NO: 376 - RNA, 25 nt, Artificial Sequence
  Description of Artificial Sequence: Synthetic oligonucleotide
uuuagugcugaaaucagcaccgugu

SEQ ID NO: 377 - RNA, 25 nt, Artificial Sequence
  Description of Artificial Sequence: Synthetic oligonucleotide
cacggugcugauuucagcacuaaag

SEQ ID NO: 378 - RNA, 25 nt, Artificial Sequence
  Description of Artificial Sequence: Synthetic oligonucleotide
cuuuagugcugaaaucagcaccgug

SEQ ID NO: 379 - RNA, 25 nt, Artificial Sequence
  Description of Artificial Sequence: Synthetic oligonucleotide
cggugcugauuucagcacuaaagau

SEQ ID NO: 380 - RNA, 25 nt, Artificial Sequence
  Description of Artificial Sequence: Synthetic oligonucleotide
aucuuuagugcugaaaucagcaccg

SEQ ID NO: 381 - RNA, 25 nt, Artificial Sequence
  Description of Artificial Sequence: Synthetic oligonucleotide
gaugcugauaaugacaacuguaugu

SEQ ID NO: 382 - RNA, 25 nt, Artificial Sequence
  Description of Artificial Sequence: Synthetic oligonucleotide
acauacaguugucauuaucagcauc

SEQ ID NO: 383 - RNA, 25 nt, Artificial Sequence
  Description of Artificial Sequence: Synthetic oligonucleotide
gcugauaaugacaacuguaugugca

SEQ ID NO: 384 - RNA, 25 nt, Artificial Sequence
  Description of Artificial Sequence: Synthetic oligonucleotide
ugcacauacaguugucauuaucagc

SEQ ID NO: 385 - RNA, 25 nt, Artificial Sequence
  Description of Artificial Sequence: Synthetic oligonucleotide
uggaauguucuauacugcgggacaa

SEQ ID NO: 386 - RNA, 25 nt, Artificial Sequence
  Description of Artificial Sequence: Synthetic oligonucleotide
uugucccgcaguauagaacauucca

SEQ ID NO: 387 - RNA, 25 nt, Artificial Sequence
  Description of Artificial Sequence: Synthetic oligonucleotide
ugaaugggauaaaguggcacuacuu

SEQ ID NO: 388 - RNA, 25 nt, Artificial Sequence
  Description of Artificial Sequence: Synthetic oligonucleotide
aaguagugccacuuuaucccauuca

SEQ ID NO: 389 - RNA, 25 nt, Artificial Sequence
  Description of Artificial Sequence: Synthetic oligonucleotide
caacuuaguagagcuaccaacaaca

SEQ ID NO: 390 - RNA, 25 nt, Artificial Sequence
  Description of Artificial Sequence: Synthetic oligonucleotide
uguuguugguagcucuacuaaguug

SEQ ID NO: 391 - RNA, 25 nt, Artificial Sequence
  Description of Artificial Sequence: Synthetic oligonucleotide
cauuucgagacugugcagauguaua

SEQ ID NO: 392 - RNA, 25 nt, Artificial Sequence
  Description of Artificial Sequence: Synthetic oligonucleotide
uauacaucugcacagucucgaaaug

SEQ ID NO: 393 - RNA, 25 nt, Artificial Sequence
  Description of Artificial Sequence: Synthetic oligonucleotide
gggaagauggaagccuggauuucca

SEQ ID NO: 394 - RNA, 25 nt, Artificial Sequence
  Description of Artificial Sequence: Synthetic oligonucleotide
uggaaauccaggcuuccaucuuccc

SEQ ID NO: 395 - RNA, 25 nt, Artificial Sequence
  Description of Artificial Sequence: Synthetic oligonucleotide
ccucuggugaauauuggcucgggaa

SEQ ID NO: 396 - RNA, 25 nt, Artificial Sequence
  Description of Artificial Sequence: Synthetic oligonucleotide
uucccgagccaauauucaccagagg

SEQ ID NO: 397 - RNA, 25 nt, Artificial Sequence
  Description of Artificial Sequence: Synthetic oligonucleotide
gaggauugagcugauggacugggaa

SEQ ID NO: 398 - RNA, 25 nt, Artificial Sequence
  Description of Artificial Sequence: Synthetic oligonucleotide
uucccaguccaucagcucaauccuc

SEQ ID NO: 399 - RNA, 25 nt, Artificial Sequence
  Description of Artificial Sequence: Synthetic oligonucleotide
cgacagauuccacauaggaaaugaa

SEQ ID NO: 400 - RNA, 25 nt, Artificial Sequence
  Description of Artificial Sequence: Synthetic oligonucleotide
uucauuuccuauguggaaucugucg

SEQ ID NO: 401 - RNA, 25 nt, Artificial Sequence
  Description of Artificial Sequence: Synthetic oligonucleotide
gcaaacagagcagcuugaucuuaca

SEQ ID NO: 402 - RNA, 25 nt, Artificial Sequence
  Description of Artificial Sequence: Synthetic oligonucleotide
uguaagaucaagcugcucuguuugc

SEQ ID NO: 403 - RNA, 25 nt, Artificial Sequence
  Description of Artificial Sequence: Synthetic oligonucleotide
cagcuugaucuuacacggugcugau

SEQ ID NO: 404 - RNA, 25 nt, Artificial Sequence
  Description of Artificial Sequence: Synthetic oligonucleotide
aucagcaccguguaagaucaagcug

SEQ ID NO: 405 - RNA, 25 nt, Artificial Sequence
  Description of Artificial Sequence: Synthetic oligonucleotide
ccuuccaaucuaaauggaauguucu

SEQ ID NO: 406 - RNA, 25 nt, Artificial Sequence
  Description of Artificial Sequence: Synthetic oligonucleotide
agaacauuccauuuagauuggaagg

SEQ ID NO: 407 - RNA, 25 nt, Artificial Sequence
  Description of Artificial Sequence: Synthetic oligonucleotide
ggcacuacuucaaagggcccaguua

SEQ ID NO: 408 - RNA, 25 nt, Artificial Sequence
  Description of Artificial Sequence: Synthetic oligonucleotide
uaacugggcccuuugaaguacugcc

SEQ ID NO: 409 - RNA, 25 nt, Artificial Sequence
  Description of Artificial Sequence: Synthetic oligonucleotide
caacaugggcaaugugccuacacuu

SEQ ID NO: 410 - RNA, 25 nt, Artificial Sequence
  Description of Artificial Sequence: Synthetic oligonucleotide
aaguguaggcacauugcccauguug

SEQ ID NO: 411 - RNA, 25 nt, Artificial Sequence
  Description of Artificial Sequence: Synthetic oligonucleotide
caugggcaaugugccuacacuuuca

SEQ ID NO: 412 - RNA, 25 nt, Artificial Sequence
  Description of Artificial Sequence: Synthetic oligonucleotide
ugaaaguguaggcacauugcccaug

SEQ ID NO: 413 - RNA, 25 nt, Artificial Sequence
  Description of Artificial Sequence: Synthetic oligonucleotide
ccuacacuuucauucuuccagaaca

SEQ ID NO: 414 - RNA, 25 nt, Artificial Sequence
  Description of Artificial Sequence: Synthetic oligonucleotide
uguucuggaagaaugaaaguguagg

SEQ ID NO: 415 - RNA, 25 nt, Artificial Sequence
  Description of Artificial Sequence: Synthetic oligonucleotide
ccagauacaacagaaugcuguucaa

SEQ ID NO: 416 - RNA, 25 nt, Artificial Sequence
  Description of Artificial Sequence: Synthetic oligonucleotide
uugaacagcauucuguuguaucugg

SEQ ID NO: 417 - RNA, 25 nt, Artificial Sequence
  Description of Artificial Sequence: Synthetic oligonucleotide
cagauguugagacccagguacuaaa

SEQ ID NO: 418 - RNA, 25 nt, Artificial Sequence
  Description of Artificial Sequence: Synthetic oligonucleotide
uuuaguaccugggucucaacaucug

SEQ ID NO: 419 - RNA, 25 nt, Artificial Sequence
  Description of Artificial Sequence: Synthetic oligonucleotide
gguacuaaaucaaacaucccgacuu

SEQ ID NO: 420 - RNA, 25 nt, Artificial Sequence
  Description of Artificial Sequence: Synthetic oligonucleotide
aagucgggauguuugauuuaguacc

SEQ ID NO: 421 - RNA, 25 nt, Artificial Sequence
  Description of Artificial Sequence: Synthetic oligonucleotide
caucccgacuugaaauacaacugcu

SEQ ID NO: 422 - RNA, 25 nt, Artificial Sequence
  Description of Artificial Sequence: Synthetic oligonucleotide
agcaguuguauuucaagucgggaug

SEQ ID NO: 423 - RNA, 25 nt, Artificial Sequence
  Description of Artificial Sequence: Synthetic oligonucleotide
gacuugaaauacaacugcuagagaa

SEQ ID NO: 424 - RNA, 25 nt, Artificial Sequence
  Description of Artificial Sequence: Synthetic oligonucleotide
uucucuagcaguuguauuucaaguc

SEQ ID NO: 425 - RNA, 25 nt, Artificial Sequence
  Description of Artificial Sequence: Synthetic oligonucleotide
cauacaagcuagagaagcaacuucu

SEQ ID NO: 426 - RNA, 25 nt, Artificial Sequence
  Description of Artificial Sequence: Synthetic oligonucleotide
agaaguugcuucucuagcuuguaug

SEQ ID NO: 427 - RNA, 25 nt, Artificial Sequence
  Description of Artificial Sequence: Synthetic oligonucleotide
gcaacuucuccaacagacaaaugaa

SEQ ID NO: 428 - RNA, 25 nt, Artificial Sequence
  Description of Artificial Sequence: Synthetic oligonucleotide
uucauuugucuguuggagaaguugc

SEQ ID NO: 429 - RNA, 25 nt, Artificial Sequence
  Description of Artificial Sequence: Synthetic oligonucleotide
uggagcucauggacacaguucauaa

SEQ ID NO: 430 - RNA, 25 nt, Artificial Sequence
  Description of Artificial Sequence: Synthetic oligonucleotide
uuaugaacuguguccaugagcucca

SEQ ID NO: 431 - RNA, 25 nt, Artificial Sequence
  Description of Artificial Sequence: Synthetic oligonucleotide
caguacgacagauuccacauaggaa

SEQ ID NO: 432 - RNA, 25 nt, Artificial Sequence
  Description of Artificial Sequence: Synthetic oligonucleotide
uuccuauguggaaucugucguacug

SEQ ID NO: 433 - RNA, 25 nt, Artificial Sequence
  Description of Artificial Sequence: Synthetic oligonucleotide
caacaugggcaaugugccuacacuu

SEQ ID NO: 434 - RNA, 25 nt, Artificial Sequence
  Description of Artificial Sequence: Synthetic oligonucleotide
aaguguaggcacauugcccauguug

SEQ ID NO: 435 - RNA, 25 nt, Artificial Sequence
  Description of Artificial Sequence: Synthetic oligonucleotide
caugggcaaugugccuacacuuuca

SEQ ID NO: 436 - RNA, 25 nt, Artificial Sequence
  Description of Artificial Sequence: Synthetic oligonucleotide
ugaaaguguaggcacauugcccaug

SEQ ID NO: 437 - RNA, 25 nt, Artificial Sequence
  Description of Artificial Sequence: Synthetic oligonucleotide
ccuacacuuucauucuuccagaaca

SEQ ID NO: 438 - RNA, 25 nt, Artificial Sequence
  Description of Artificial Sequence: Synthetic oligonucleotide
uguucuggaagaaugaaaguguagg

SEQ ID NO: 439 - RNA, 25 nt, Artificial Sequence
  Description of Artificial Sequence: Synthetic oligonucleotide
ggagaagauauaaccggauucaaca

SEQ ID NO: 440 - RNA, 25 nt, Artificial Sequence
  Description of Artificial Sequence: Synthetic oligonucleotide
uguugaauccgguuauaucuucucc

SEQ ID NO: 441 - RNA, 25 nt, Artificial Sequence
  Description of Artificial Sequence: Synthetic oligonucleotide
gauauaaccggauucaacaugggca

SEQ ID NO: 442 - RNA, 25 nt, Artificial Sequence
  Description of Artificial Sequence: Synthetic oligonucleotide
ugcccauguugaauccgguuauauc

SEQ ID NO: 443 - RNA, 25 nt, Artificial Sequence
  Description of Artificial Sequence: Synthetic oligonucleotide
ucaacaugggcaaugugccuacacu

SEQ ID NO: 444 - RNA, 25 nt, Artificial Sequence
  Description of Artificial Sequence: Synthetic oligonucleotide
aguguaggcacauugcccauguuga

SEQ ID NO: 445 - RNA, 25 nt, Artificial Sequence
  Description of Artificial Sequence: Synthetic oligonucleotide
cagauguugagacccagguacuaaa

SEQ ID NO: 446 - RNA, 25 nt, Artificial Sequence
  Description of Artificial Sequence: Synthetic oligonucleotide
uuuaguaccugggucucaacaucug

SEQ ID NO: 447 - RNA, 25 nt, Artificial Sequence
  Description of Artificial Sequence: Synthetic oligonucleotide
gacagauuccacauaggaaaugaaa

SEQ ID NO: 448 - RNA, 25 nt, Artificial Sequence
  Description of Artificial Sequence: Synthetic oligonucleotide
uuucauuuccuauguggaaucuguc

SEQ ID NO: 449 - RNA, 25 nt, Artificial Sequence
  Description of Artificial Sequence: Synthetic oligonucleotide
ugaaugggauaaaguggcacuacuu

SEQ ID NO: 450 - RNA, 25 nt, Artificial Sequence
  Description of Artificial Sequence: Synthetic oligonucleotide
aaguagugccacuuuaucccauuca

SEQ ID NO: 451 - RNA, 25 nt, Artificial Sequence
  Description of Artificial Sequence: Synthetic oligonucleotide
ugaaugggauaaaguggcacuacuu

SEQ ID NO: 452 - RNA, 25 nt, Artificial Sequence
  Description of Artificial Sequence: Synthetic oligonucleotide
aaguagugccacuuuaucccauuca

SEQ ID NO: 453 - RNA, 25 nt, Artificial Sequence
  Description of Artificial Sequence: Synthetic oligonucleotide
ggcacuacuucaaagggcccaguua

SEQ ID NO: 454 - RNA, 25 nt, Artificial Sequence
  Description of Artificial Sequence: Synthetic oligonucleotide
uaacugggcccuuugaaguagugcc

SEQ ID NO: 455 - RNA, 25 nt, Artificial Sequence
  Description of Artificial Sequence: Synthetic oligonucleotide
ccacuguugcuaaagaagaacaaau

SEQ ID NO: 456 - RNA, 25 nt, Artificial Sequence
  Description of Artificial Sequence: Synthetic oligonucleotide
auuuguucuucuuuagcaacagugg

SEQ ID NO: 457 - RNA, 25 nt, Artificial Sequence
  Description of Artificial Sequence: Synthetic oligonucleotide
cagcuucagagacugugcugaagua

SEQ ID NO: 458 - RNA, 25 nt, Artificial Sequence
  Description of Artificial Sequence: Synthetic oligonucleotide
uacuucagcacagucucugaagcug

SEQ ID NO: 459 - RNA, 25 nt, Artificial Sequence
  Description of Artificial Sequence: Synthetic oligonucleotide
ggacacaccacaaauggcaucuaca

SEQ ID NO: 460 - RNA, 25 nt, Artificial Sequence
  Description of Artificial Sequence: Synthetic oligonucleotide
uguagaugccauuuguggugugucc

SEQ ID NO: 461 - RNA, 25 nt, Artificial Sequence
  Description of Artificial Sequence: Synthetic oligonucleotide
caucuacacguuaacauucccuaau

SEQ ID NO: 462 - RNA, 25 nt, Artificial Sequence
  Description of Artificial Sequence: Synthetic oligonucleotide
auuagggaauguuaacguguagaug

SEQ ID NO: 463 - RNA, 25 nt, Artificial Sequence
  Description of Artificial Sequence: Synthetic oligonucleotide
uggaggaggcggguggacaauuauu

SEQ ID NO: 464 - RNA, 25 nt, Artificial Sequence
  Description of Artificial Sequence: Synthetic oligonucleotide
aauaauuguccacccgccuccucca

SEQ ID NO: 465 - RNA, 25 nt, Artificial Sequence
  Description of Artificial Sequence: Synthetic oligonucleotide
gguggacaauuauucagcgacguga

SEQ ID NO: 466 - RNA, 25 nt, Artificial Sequence
  Description of Artificial Sequence: Synthetic oligonucleotide
ucacgucgcugaauaauuguccacc

SEQ ID NO: 467 - RNA, 25 nt, Artificial Sequence
  Description of Artificial Sequence: Synthetic oligonucleotide
cgcaacugacuaaucagcaacgcua

SEQ ID NO: 468 - RNA, 25 nt, Artificial Sequence
  Description of Artificial Sequence: Synthetic oligonucleotide
uagcguugcugauuagucaguugcg

SEQ ID NO: 469 - RNA, 25 nt, Artificial Sequence
  Description of Artificial Sequence: Synthetic oligonucleotide
cagcaucagccaaccaggaaaugau

SEQ ID NO: 470 - RNA, 25 nt, Artificial Sequence
  Description of Artificial Sequence: Synthetic oligonucleotide
aucauuuccugguuggcugaugcug

SEQ ID NO: 471 - RNA, 25 nt, Artificial Sequence
  Description of Artificial Sequence: Synthetic oligonucleotide
ccuuccaacuugaacggaauguacu

SEQ ID NO: 472 - RNA, 25 nt, Artificial Sequence
  Description of Artificial Sequence: Synthetic oligonucleotide
aguacauuccguucaaguuggaagg

SEQ ID NO: 473 - RNA, 25 nt, Artificial Sequence
  Description of Artificial Sequence: Synthetic oligonucleotide
cagaacacaaauaaguucaacggca

SEQ ID NO: 474 - RNA, 25 nt, Artificial Sequence
  Description of Artificial Sequence: Synthetic oligonucleotide
ugccguugaacuuauuuguguucug

SEQ ID NO: 475 - RNA, 25 nt, Artificial Sequence
  Description of Artificial Sequence: Synthetic oligonucleotide
gaucuugucuuggccgcagccuaua

SEQ ID NO: 476 - RNA, 25 nt, Artificial Sequence
  Description of Artificial Sequence: Synthetic oligonucleotide
uauaggcugcggccaagacaagauc

SEQ ID NO: 477 - RNA, 25 nt, Artificial Sequence
  Description of Artificial Sequence: Synthetic oligonucleotide
ccgcagccuauaacaacuuucggaa

SEQ ID NO: 478 - RNA, 25 nt, Artificial Sequence
  Description of Artificial Sequence: Synthetic oligonucleotide
uuccgaaaguuguuauaggcugcgg

SEQ ID NO: 479 - RNA, 25 nt, Artificial Sequence
  Description of Artificial Sequence: Synthetic oligonucleotide
caagugcuggagaacaucauggaaa

SEQ ID NO: 480 - RNA, 25 nt, Artificial Sequence
  Description of Artificial Sequence: Synthetic oligonucleotide
uuuccaugauguucuccagcacuug

SEQ ID NO: 481 - RNA, 25 nt, Artificial Sequence
  Description of Artificial Sequence: Synthetic oligonucleotide
ggacaacaugaagaaagaaauggua

SEQ ID NO: 482 - RNA, 25 nt, Artificial Sequence
  Description of Artificial Sequence: Synthetic oligonucleotide
uaccauuucuuucuucauguugucc

SEQ ID NO: 483 - RNA, 25 nt, Artificial Sequence
  Description of Artificial Sequence: Synthetic oligonucleotide
ccuguugaaccaaacagcugagcaa

SEQ ID NO: 484 - RNA, 25 nt, Artificial Sequence
  Description of Artificial Sequence: Synthetic oligonucleotide
uugcucagcuguuugguucaacagg

SEQ ID NO: 485 - RNA, 25 nt, Artificial Sequence
  Description of Artificial Sequence: Synthetic oligonucleotide
uaacugauguggaagcccaaguauu

SEQ ID NO: 486 - RNA, 25 nt, Artificial Sequence
  Description of Artificial Sequence: Synthetic oligonucleotide
aauacuugggcuuccacaucaguua

SEQ ID NO: 487 - RNA, 25 nt, Artificial Sequence
  Description of Artificial Sequence: Synthetic oligonucleotide
ccacgagacuugaacuucagcucuu

SEQ ID NO: 488 - RNA, 25 nt, Artificial Sequence
  Description of Artificial Sequence: Synthetic oligonucleotide
aagagcugaaguucaagucucgugg

SEQ ID NO: 489 - RNA, 25 nt, Artificial Sequence
  Description of Artificial Sequence: Synthetic oligonucleotide
accacaaauggcaucuacacguuaa

SEQ ID NO: 490 - RNA, 25 nt, Artificial Sequence
  Description of Artificial Sequence: Synthetic oligonucleotide
uuaacguguagaugccauuuguggu

SEQ ID NO: 491 - RNA, 25 nt, Artificial Sequence
  Description of Artificial Sequence: Synthetic oligonucleotide
cacguuaacauucccuaauucuaca

SEQ ID NO: 492 - RNA, 25 nt, Artificial Sequence
  Description of Artificial Sequence: Synthetic oligonucleotide
uguagaauuagggaauguuaacgug

SEQ ID NO: 493 - RNA, 25 nt, Artificial Sequence
  Description of Artificial Sequence: Synthetic oligonucleotide
gggauuugguaacccuucaggagaa

SEQ ID NO: 494 - RNA, 25 nt, Artificial Sequence
  Description of Artificial Sequence: Synthetic oligonucleotide
uucuccugaaggguuaccaaauccc

SEQ ID NO: 495 - RNA, 25 nt, Artificial Sequence
  Description of Artificial Sequence: Synthetic oligonucleotide
gaugcaugugguccuuccaacuuga

SEQ ID NO: 496 - RNA, 25 nt, Artificial Sequence
  Description of Artificial Sequence: Synthetic oligonucleotide
ucaaguuggaaggaccacaugcauc

SEQ ID NO: 497 - RNA, 25 nt, Artificial Sequence
  Description of Artificial Sequence: Synthetic oligonucleotide
cggcauuaaaugguacuacuggaaa

SEQ ID NO: 498 - RNA, 25 nt, Artificial Sequence
  Description of Artificial Sequence: Synthetic oligonucleotide
uuuccaguaguaccauuuaaugccg

SEQ ID NO: 499 - RNA, 25 nt, Artificial Sequence
  Description of Artificial Sequence: Synthetic oligonucleotide
ucuggacguguguuugcccucaagu

SEQ ID NO: 500 - RNA, 25 nt, Artificial Sequence
  Description of Artificial Sequence: Synthetic oligonucleotide
acuugagggcaaacacacguccaga

SEQ ID NO: 501 - RNA, 25 nt, Artificial Sequence
  Description of Artificial Sequence: Synthetic oligonucleotide
uggacguguguuugcccucaaguuu

SEQ ID NO: 502 - RNA, 25 nt, Artificial Sequence
  Description of Artificial Sequence: Synthetic oligonucleotide
aaacuugagggcaaacacacgucca

SEQ ID NO: 503 - RNA, 25 nt, Artificial Sequence
  Description of Artificial Sequence: Synthetic oligonucleotide
ggacguguguuugcccucaaguuug

SEQ ID NO: 504 - RNA, 25 nt, Artificial Sequence
  Description of Artificial Sequence: Synthetic oligonucleotide
caaacuugagggcaaauauaugucc

SEQ ID NO: 505 - RNA, 25 nt, Artificial Sequence
  Description of Artificial Sequence: Synthetic oligonucleotide
acugaagaaagaauguggcagauug

SEQ ID NO: 506 - RNA, 25 nt, Artificial Sequence
  Description of Artificial Sequence: Synthetic oligonucleotide
caaucugccacauucuuucuucagu

SEQ ID NO: 507 - RNA, 25 nt, Artificial Sequence
  Description of Artificial Sequence: Synthetic oligonucleotide
gaagaaagaauguggcagauuguuu

SEQ ID NO: 508 - RNA, 25 nt, Artificial Sequence
  Description of Artificial Sequence: Synthetic oligonucleotide
aaacaaucugccacauucuuucuuc

SEQ ID NO: 509 - RNA, 25 nt, Artificial Sequence
  Description of Artificial Sequence: Synthetic oligonucleotide
ugaucuugucuuggccgcagccuau

SEQ ID NO: 510 - RNA, 25 nt, Artificial Sequence
  Description of Artificial Sequence: Synthetic oligonucleotide
auaggcugcggccaagacaagauca

SEQ ID NO: 511 - RNA, 25 nt, Artificial Sequence
  Description of Artificial Sequence: Synthetic oligonucleotide
gccgcagccuauaacaacuuucgga

SEQ ID NO: 512 - RNA, 25 nt, Artificial Sequence
  Description of Artificial Sequence: Synthetic oligonucleotide
uccgaaaguuguuauaggcugcggc

SEQ ID NO: 513 - RNA, 25 nt, Artificial Sequence
  Description of Artificial Sequence: Synthetic oligonucleotide
ccuauaacaacuuucggaagagcau

SEQ ID NO: 514 - RNA, 25 nt, Artificial Sequence
  Description of Artificial Sequence: Synthetic oligonucleotide
augcucuuccgaaaguuguuauagg

SEQ ID NO: 515 - RNA, 25 nt, Artificial Sequence
  Description of Artificial Sequence: Synthetic oligonucleotide
caguggcuaaugaagcuugagaauu

SEQ ID NO: 516 - RNA, 25 nt, Artificial Sequence
  Description of Artificial Sequence: Synthetic oligonucleotide
aauucucaagcuucauuagccacug

SEQ ID NO: 517 - RNA, 25 nt, Artificial Sequence
  Description of Artificial Sequence: Synthetic oligonucleotide
aguggcuaaugaagcuugagaauua

SEQ ID NO: 518 - RNA, 25 nt, Artificial Sequence
  Description of Artificial Sequence: Synthetic oligonucleotide
uaauucucaagcuucauuagccacu

SEQ ID NO: 519 - RNA, 25 nt, Artificial Sequence
  Description of Artificial Sequence: Synthetic oligonucleotide
aaccagacggcugugaugauagaaa

SEQ ID NO: 520 - RNA, 25 nt, Artificial Sequence
  Description of Artificial Sequence: Synthetic oligonucleotide
uuucuaucaucacagccgucugguu

SEQ ID NO: 521 - RNA, 25 nt, Artificial Sequence
  Description of Artificial Sequence: Synthetic oligonucleotide
ccagacggcugugaugauagaaaua

SEQ ID NO: 522 - RNA, 25 nt, Artificial Sequence
  Description of Artificial Sequence: Synthetic oligonucleotide
uauuucuaucaucacagccgucugg

SEQ ID NO: 523 - RNA, 25 nt, Artificial Sequence
  Description of Artificial Sequence: Synthetic oligonucleotide
acagcugagcaaacgcggaaguuaa

SEQ ID NO: 524 - RNA, 25 nt, Artificial Sequence
  Description of Artificial Sequence: Synthetic oligonucleotide
uuaacuuccgcguuugcucagcugu

SEQ ID NO: 525 - RNA, 25 nt, Artificial Sequence
  Description of Artificial Sequence: Synthetic oligonucleotide
aacgcggaaguuaacugauguggaa

SEQ ID NO: 526 - RNA, 25 nt, Artificial Sequence
  Description of Artificial Sequence: Synthetic oligonucleotide
uuccacaucaguuaacuuccgcguu

SEQ ID NO: 527 - RNA, 25 nt, Artificial Sequence
  Description of Artificial Sequence: Synthetic oligonucleotide
ggaaguuaacugauguggaagccca

SEQ ID NO: 528 - RNA, 25 nt, Artificial Sequence
  Description of Artificial Sequence: Synthetic oligonucleotide
ugggcuuccacaucaguuaacuucc

SEQ ID NO: 529 - RNA, 25 nt, Artificial Sequence
  Description of Artificial Sequence: Synthetic oligonucleotide
gaaguuaacugauguggaagcccaa

SEQ ID NO: 530 - RNA, 25 nt, Artificial Sequence
  Description of Artificial Sequence: Synthetic oligonucleotide
uugggcuuccacaucaguuaacuuc

SEQ ID NO: 531 - RNA, 25 nt, Artificial Sequence
  Description of Artificial Sequence: Synthetic oligonucleotide
acugauguggaagcccaaguauuaa

SEQ ID NO: 532 - RNA, 25 nt, Artificial Sequence
  Description of Artificial Sequence: Synthetic oligonucleotide
uuaauacuugggcuuccacaucagu

SEQ ID NO: 533 - RNA, 25 nt, Artificial Sequence
  Description of Artificial Sequence: Synthetic oligonucleotide
aguauuaaaucagaccacgagacuu

SEQ ID NO: 534 - RNA, 25 nt, Artificial Sequence
  Description of Artificial Sequence: Synthetic oligonucleotide
aagucucguggucugauuuaauacu

SEQ ID NO: 535 - RNA, 25 nt, Artificial Sequence
  Description of Artificial Sequence: Synthetic oligonucleotide
ggaacacucccucucgacaaacaaa

SEQ ID NO: 536 - RNA, 25 nt, Artificial Sequence
  Description of Artificial Sequence: Synthetic oligonucleotide
uuuguuugucgagagggaguguucc

SEQ ID NO: 537 - RNA, 25 nt, Artificial Sequence
  Description of Artificial Sequence: Synthetic oligonucleotide
uggaccagaccagugaaauaaacaa

SEQ ID NO: 538 - RNA, 25 nt, Artificial Sequence
  Description of Artificial Sequence: Synthetic oligonucleotide
uuguuuauuucacuggucuggucca

SEQ ID NO: 539 - RNA, 25 nt, Artificial Sequence
  Description of Artificial Sequence: Synthetic oligonucleotide
cccacuguugcuaaagaagaacaaa

SEQ ID NO: 540 - RNA, 25 nt, Artificial Sequence
  Description of Artificial Sequence: Synthetic oligonucleotide
uuuguucuucuuuagcaacaguggg

SEQ ID NO: 541 - RNA, 25 nt, Artificial Sequence
  Description of Artificial Sequence: Synthetic oligonucleotide
gcuaaagaagaacaaaucagcuuca

SEQ ID NO: 542 - RNA, 25 nt, Artificial Sequence
  Description of Artificial Sequence: Synthetic oligonucleotide
ugaagcugauuuguucuucuuuagc

SEQ ID NO: 543 - RNA, 25 nt, Artificial Sequence
  Description of Artificial Sequence: Synthetic oligonucleotide
caccacaaauggcaucuacacguua

SEQ ID NO: 544 - RNA, 25 nt, Artificial Sequence
  Description of Artificial Sequence: Synthetic oligonucleotide
uaacguguagaugccauuuguggug

SEQ ID NO: 545 - RNA, 25 nt, Artificial Sequence
  Description of Artificial Sequence: Synthetic oligonucleotide
caaauggcaucuacacguuaacauu

SEQ ID NO: 546 - RNA, 25 nt, Artificial Sequence
  Description of Artificial Sequence: Synthetic oligonucleotide
aauguuaacguguagaugccauuug

SEQ ID NO: 547 - RNA, 25 nt, Artificial Sequence
  Description of Artificial Sequence: Synthetic oligonucleotide
gaucaaggccuacugugacauggaa

SEQ ID NO: 548 - RNA, 25 nt, Artificial Sequence
  Description of Artificial Sequence: Synthetic oligonucleotide
uuccaugucacaguaggccuugauc

SEQ ID NO: 549 - RNA, 25 nt, Artificial Sequence
  Description of Artificial Sequence: Synthetic oligonucleotide
gaggaggcggguggacaauuauuca

SEQ ID NO: 550 - RNA, 25 nt, Artificial Sequence
  Description of Artificial Sequence: Synthetic oligonucleotide
ugaauaauuguccacccgccuccuc

SEQ ID NO: 551 - RNA, 25 nt, Artificial Sequence
  Description of Artificial Sequence: Synthetic oligonucleotide
gacgugaggauggcagcguugauuu

SEQ ID NO: 552 - RNA, 25 nt, Artificial Sequence
  Description of Artificial Sequence: Synthetic oligonucleotide
aaaucaacgcugccauccucacguc

SEQ ID NO: 553 - RNA, 25 nt, Artificial Sequence
  Description of Artificial Sequence: Synthetic oligonucleotide
ggaaaugaguuuguuucgcaacuga

SEQ ID NO: 554 - RNA, 25 nt, Artificial Sequence
  Description of Artificial Sequence: Synthetic oligonucleotide
ucaguugcgaaacaaacucauuucc

SEQ ID NO: 555 - RNA, 25 nt, Artificial Sequence
  Description of Artificial Sequence: Synthetic oligonucleotide
gaaaugaguuuguuucgcaacugac

SEQ ID NO: 556 - RNA, 25 nt, Artificial Sequence
  Description of Artificial Sequence: Synthetic oligonucleotide
gucaguugcgaaacaaacucauuuc

SEQ ID NO: 557 - RNA, 25 nt, Artificial Sequence
  Description of Artificial Sequence: Synthetic oligonucleotide
gaaugaggcuuacucauuguaugaa

SEQ ID NO: 558 - RNA, 25 nt, Artificial Sequence
  Description of Artificial Sequence: Synthetic oligonucleotide
uucauacaaugaguaagccucauuc

SEQ ID NO: 559 - RNA, 25 nt, Artificial Sequence
  Description of Artificial Sequence: Synthetic oligonucleotide
gaggcuuacucauuguaugaacauu

SEQ ID NO: 560 - RNA, 25 nt, Artificial Sequence
  Description of Artificial Sequence: Synthetic oligonucleotide
aauguucauacaaugaguaagccuc

SEQ ID NO: 561 - RNA, 25 nt, Artificial Sequence
  Description of Artificial Sequence: Synthetic oligonucleotide
acaaaggauggagacaacgacaaau

SEQ ID NO: 562 - RNA, 25 nt, Artificial Sequence
  Description of Artificial Sequence: Synthetic oligonucleotide
auuugucguugucuccauccuuugu

SEQ ID NO: 563 - RNA, 25 nt, Artificial Sequence
  Description of Artificial Sequence: Synthetic oligonucleotide
aggauggagacaacgacaaauguau

SEQ ID NO: 564 - RNA, 25 nt, Artificial Sequence
  Description of Artificial Sequence: Synthetic oligonucleotide
auacauuugucguugucuccauccu

SEQ ID NO: 565 - RNA, 25 nt, Artificial Sequence
  Description of Artificial Sequence: Synthetic oligonucleotide
gagacaacgacaaauguauuugcaa

SEQ ID NO: 566 - RNA, 25 nt, Artificial Sequence
  Description of Artificial Sequence: Synthetic oligonucleotide
uugcaaauacauuugucguugucuc

SEQ ID NO: 567 - RNA, 25 nt, Artificial Sequence
  Description of Artificial Sequence: Synthetic oligonucleotide
caacuugaacggaauguacuaucca

SEQ ID NO: 568 - RNA, 25 nt, Artificial Sequence
  Description of Artificial Sequence: Synthetic oligonucleotide
uggauaguacauuccguucaaguug

SEQ ID NO: 569-588 - skipped (20 entries)

SEQ ID NO: 589 - RNA, 25 nt, Artificial Sequence
  Description of Artificial Sequence: Synthetic oligonucleotide
gaacacaaauaaguucaacggcauu

SEQ ID NO: 590 - RNA, 25 nt, Artificial Sequence
  Description of Artificial Sequence: Synthetic oligonucleotide
aaugccguugaacuuauuuguguuc

SEQ ID NO: 591 - RNA, 25 nt, Artificial Sequence
  Description of Artificial Sequence: Synthetic oligonucleotide
gguacuacuggaaaggcucaggcua

SEQ ID NO: 592 - RNA, 25 nt, Artificial Sequence
  Description of Artificial Sequence: Synthetic oligonucleotide
uagccugagccuuuccaguaguacc

SEQ ID NO: 593 - RNA, 25 nt, Artificial Sequence
  Description of Artificial Sequence: Synthetic oligonucleotide
uacuacuggaaaggcucaggcuauu

SEQ ID NO: 594 - RNA, 25 nt, Artificial Sequence
  Description of Artificial Sequence: Synthetic oligonucleotide
aauagccugagccuuuccaguagua

SEQ ID NO: 595 - RNA, 25 nt, Artificial Sequence
  Description of Artificial Sequence: Synthetic oligonucleotide
uggaaaggcucaggcuauucgcuca

SEQ ID NO: 596 - RNA, 25 nt, Artificial Sequence
  Description of Artificial Sequence: Synthetic oligonucleotide
ugagcgaauagccugagccuuucca

SEQ ID NO: 597 - RNA, 25 nt, Artificial Sequence
  Description of Artificial Sequence: Synthetic oligonucleotide
cacaaccaugaugauccgaccagca

SEQ ID NO: 598 - RNA, 25 nt, Artificial Sequence
  Description of Artificial Sequence: Synthetic oligonucleotide
ugcuggucggaucaucaugguugug

SEQ ID NO: 599 - RNA, 25 nt, Artificial Sequence
  Description of Artificial Sequence: Synthetic oligonucleotide
aagacuuaagcccagugcacugaaa

SEQ ID NO: 600 - RNA, 25 nt, Artificial Sequence
  Description of Artificial Sequence: Synthetic oligonucleotide
uuucagugcacugggcuuaagucuu

SEQ ID NO: 601 - RNA, 25 nt, Artificial Sequence
  Description of Artificial Sequence: Synthetic oligonucleotide
ccacaugcuccagauuagagccugu

SEQ ID NO: 602 - RNA, 25 nt, Artificial Sequence
  Description of Artificial Sequence: Synthetic oligonucleotide
acaggcucuaaucuggagcaugugg

SEQ ID NO: 603 - RNA, 25 nt, Artificial Sequence
  Description of Artificial Sequence: Synthetic oligonucleotide
cacaugcuccagauuagagccugua

SEQ ID NO: 604 - RNA, 25 nt, Artificial Sequence
  Description of Artificial Sequence: Synthetic oligonucleotide
uacaggcucuaaucuggagcaugug

SEQ ID NO: 605 - RNA, 25 nt, Artificial Sequence
  Description of Artificial Sequence: Synthetic oligonucleotide
caugcuccagauuagagccuguaaa

SEQ ID NO: 606 - RNA, 25 nt, Artificial Sequence
  Description of Artificial Sequence: Synthetic oligonucleotide
uuuacaggcucuaaucuggagcaug

SEQ ID NO: 607 - RNA, 25 nt, Artificial Sequence
  Description of Artificial Sequence: Synthetic oligonucleotide
uccagauuagagccuguaaacuuua

SEQ ID NO: 608 - RNA, 25 nt, Artificial Sequence
  Description of Artificial Sequence: Synthetic oligonucleotide
uaaaguuuacaggcucuaaucugga

SEQ ID NO: 609 - RNA, 25 nt, Artificial Sequence
  Description of Artificial Sequence: Synthetic oligonucleotide
gcagcuucuccaacauucuauuucu

SEQ ID NO: 610 - RNA, 25 nt, Artificial Sequence
  Description of Artificial Sequence: Synthetic oligonucleotide
agaaauagaauguuggagaagcugc

SEQ ID NO: 611 - RNA, 25 nt, Artificial Sequence
  Description of Artificial Sequence: Synthetic oligonucleotide
cggucaacaacucgcuccuucagaa

SEQ ID NO: 612 - RNA, 25 nt, Artificial Sequence
  Description of Artificial Sequence: Synthetic oligonucleotide
uucugaaggagcgaguuguugaccg

SEQ ID NO: 613 - RNA, 25 nt, Artificial Sequence
  Description of Artificial Sequence: Synthetic oligonucleotide
ccgucaacagcuugcugaccaugau

SEQ ID NO: 614 - RNA, 25 nt, Artificial Sequence
  Description of Artificial Sequence: Synthetic oligonucleotide
aucauggucagcaagcuguugacgg

SEQ ID NO: 615 - RNA, 25 nt, Artificial Sequence
  Description of Artificial Sequence: Synthetic oligonucleotide
gagaagauggcaguguggacuucca

SEQ ID NO: 616 - RNA, 25 nt, Artificial Sequence
  Description of Artificial Sequence: Synthetic oligonucleotide
uggaaguccacacugccaucuucuc

SEQ ID NO: 617 - RNA, 25 nt, Artificial Sequence
  Description of Artificial Sequence: Synthetic oligonucleotide
ggcaaugaguuugucucccagcuga

SEQ ID NO: 618 - RNA, 25 nt, Artificial Sequence
  Description of Artificial Sequence: Synthetic oligonucleotide
ucagcugggagacaaacucauugcc

SEQ ID NO: 619 - RNA, 25 nt, Artificial Sequence
  Description of Artificial Sequence: Synthetic oligonucleotide
gcuacgugcuuaagauccagcugaa

SEQ ID NO: 620 - RNA, 25 nt, Artificial Sequence
  Description of Artificial Sequence: Synthetic oligonucleotide
uucagcuggaucuuaagcacguagc

SEQ ID NO: 621 - RNA, 25 nt, Artificial Sequence
  Description of Artificial Sequence: Synthetic oligonucleotide
gcguaagcgacauacuagugaagau

SEQ ID NO: 622 - RNA, 25 nt, Artificial Sequence
  Description of Artificial Sequence: Synthetic oligonucleotide
aucuucacuaguaugucgcuuacgc

SEQ ID NO: 623 - RNA, 25 nt, Artificial Sequence
  Description of Artificial Sequence: Synthetic oligonucleotide
uagcaucagccaaccaggaagugau

SEQ ID NO: 624 - RNA, 25 nt, Artificial Sequence
  Description of Artificial Sequence: Synthetic oligonucleotide
aucacuuccugguuggcugaugcua

SEQ ID NO: 625 - RNA, 25 nt, Artificial Sequence
  Description of Artificial Sequence: Synthetic oligonucleotide
aaugacaaaugcaucugcaaguguu

SEQ ID NO: 626 - RNA, 25 nt, Artificial Sequence
  Description of Artificial Sequence: Synthetic oligonucleotide
aacacuugcagaugcauuugucauu

SEQ ID NO: 627 - RNA, 25 nt, Artificial Sequence
  Description of Artificial Sequence: Synthetic oligonucleotide
ccuuccaacuugaauggacaguacu

SEQ ID NO: 628 - RNA, 25 nt, Artificial Sequence
  Description of Artificial Sequence: Synthetic oligonucleotide
aguacuguccauucaaguuggaagg

SEQ ID NO: 629 - RNA, 25 nt, Artificial Sequence
  Description of Artificial Sequence: Synthetic oligonucleotide
cagcuucuccaacauucuauuucua

SEQ ID NO: 630 - RNA, 25 nt, Artificial Sequence
  Description of Artificial Sequence: Synthetic oligonucleotide
uagaaauagaauguuggagaagcug

SEQ ID NO: 631 - RNA, 25 nt, Artificial Sequence
  Description of Artificial Sequence: Synthetic oligonucleotide
cagcaugaccuaauggagaccguca

SEQ ID NO: 632 - RNA, 25 nt, Artificial Sequence
  Description of Artificial Sequence: Synthetic oligonucleotide
ugacggucuccauuaggucaugcug

SEQ ID NO: 633 - RNA, 25 nt, Artificial Sequence
  Description of Artificial Sequence: Synthetic oligonucleotide
caagagcucgguugcuauccguaaa

SEQ ID NO: 634 - RNA, 25 nt, Artificial Sequence
  Description of Artificial Sequence: Synthetic oligonucleotide
uuuacggauagcaaccgagcucuug

SEQ ID NO: 635 - RNA, 25 nt, Artificial Sequence
  Description of Artificial Sequence: Synthetic oligonucleotide
gacgcaugugguccuuccaacuuga

SEQ ID NO: 636 - RNA, 25 nt, Artificial Sequence
  Description of Artificial Sequence: Synthetic oligonucleotide
ucaaguuggaaggaccacaugcguc

SEQ ID NO: 637 - RNA, 25 nt, Artificial Sequence
  Description of Artificial Sequence: Synthetic oligonucleotide
gagaucaaggccuacugugacaugg

SEQ ID NO: 638 - RNA, 25 nt, Artificial Sequence
  Description of Artificial Sequence: Synthetic oligonucleotide
ccaugucacaguaggccuugaucuc

SEQ ID NO: 639 - RNA, 25 nt, Artificial Sequence
  Description of Artificial Sequence: Synthetic oligonucleotide
agaucaaggccuacugugacaugga

SEQ ID NO: 640 - RNA, 25 nt, Artificial Sequence
  Description of Artificial Sequence: Synthetic oligonucleotide
uccaugucacaguaggccuugaucu

SEQ ID NO: 641 - RNA, 25 nt, Artificial Sequence
  Description of Artificial Sequence: Synthetic oligonucleotide
ucgcucaaggccacaaccaugauga

SEQ ID NO: 642 - RNA, 25 nt, Artificial Sequence
  Description of Artificial Sequence: Synthetic oligonucleotide
ucaucaugguuguggccuugagcga

SEQ ID NO: 643 - RNA, 25 nt, Artificial Sequence
  Description of Artificial Sequence: Synthetic oligonucleotide
cgcucaaggccacaaccaugaugau

SEQ ID NO: 644 - RNA, 25 nt, Artificial Sequence
  Description of Artificial Sequence: Synthetic oligonucleotide
aucaucaugguuguggccuugagcg

SEQ ID NO: 645 - RNA, 25 nt, Artificial Sequence
  Description of Artificial Sequence: Synthetic oligonucleotide
gcucaaggccacaaccaugaugauc

SEQ ID NO: 646 - RNA, 25 nt, Artificial Sequence
  Description of Artificial Sequence: Synthetic oligonucleotide
gaucaucaugguuguggccuugagc

SEQ ID NO: 647 - RNA, 25 nt, Artificial Sequence
  Description of Artificial Sequence: Synthetic oligonucleotide
cucaaggccacaaccaugaugaucc

SEQ ID NO: 648 - RNA, 25 nt, Artificial Sequence
  Description of Artificial Sequence: Synthetic oligonucleotide
ggaucaucaugguuguggccuugag

SEQ ID NO: 649 - DNA, 4817 bp, Homo sapiens
agtttcccgcctatgagaggatacccctattgtttctgaaaatgctgaccgggacccacacttccaacaaaaattcctctgcccctacagcagcagcaaaagcagcagcagaagcaacagcaacagataagtgttttgatgaattgcgagatggatagggcttgagtgcccccagccctgctgataccaaatgcctttaagatacagcctttcccatcctaatctacaaaggaaacaggaaaaaggaacttaaaactccctgtgctcagacagaaatgagactgttacagcctgcttctgtgctgttccttcttgcctctaacttgtaaacaagacgtagtaggacgatgctaatggaaagtcacaaaccgctgggtttttgaaaggatccttgggacctcatgcacatttgtggaaactggatggagagatttggggaagcatggactctttagccagcttagttctctgtggagtcagcttgctcctttctggaactgtggaaggtgccatggacttgatcttgatcaattccctacctcttgtatctgatgctgaaacatctctcacctgcattgcctctgggtggcgcccccatgagcccatcaccataggaagggactttgaagccttaatgaaccagcaccaggatccgctggaagttactcaagatgtgaccagagaatgggctaaaaaagttgtttggaagagagaaaaggctagtaagatcaatggtgcttatttctgtgaagggcgagttcgaggagaggcaatcaggatacgaaccatgaagatgcgtcaacaagcttccttcctaccagctactttaactatgactgtggacaagggagataacgtgaacatatctttcaaaaaggtattgattaaagaagaagatgcagtgatttacaaaaatggttccttcatccattcagtgccccggcatgaagtacctgatattctagaagtacacctgcctcatgctcagccccaggatgctggagtgtactcggccaggtatataggaggaaacctcttcacctcggccttcaccaggctgatagtccggagatgtgaagcccagaagtggggacctgaatgcaaccatctctgtactgcttgtatgaacaatggtgtctgccatgaagatactggagaatgcatttgccctcctgggtttatgggaaggacgtgtgagaaggcttgtgaactgcacacgtttggcagaacttgtaaagaaaggtgcagtggacaagagggatgcaagtcttatgtgttctgtctccctgacccctatgggtgttcctgtgccacaggctggaagggtctgcagtgcaatgaagcatgccaccctggtttttacgggccagattgtaagcttaggtgcagctgcaacaatggggagatgtgtgatcgcttccaaggatgtctctgctctccaggatggcaggggctccagtgtgagagagaaggcataccgaggatgaccccaaagatagtggatttgccagatcatatagaagtaaacagtggtaaatttaatcccatttgcaaagcttctggctggccgctacctactaatgaagaaatgaccctggtgaagccggatgggacagtgctccatccaaaagactttaaccatacggatcatttctcagtagccatattcaccatccaccggatcctcccccctgactcaggagtttgggtctgcagtgtgaacacagtggctgggatggtggaaaagcccttcaacatttctgttaaagttcttccaaagcccctgaatgccccaaacgtgattgacactggacataactttgctgtcatcaacatcagctctgagccttactttggggatggaccaatcaaatccaagaagcttctatacaaacccgttaatcactatgaggcttggcaacatattcaagtgacaaatgagattgttacactcaactatttggaacctcggacagaatatgaactctgtgtgcaactggtccgtcgtggagagggtggggaagggcatcctggacctgtgagacgcttcacaacagcttctatcggactccctcctccaagaggtctaaatctcctgcctaaaagtcagaccactctaaatttgacctggcaaccaatatttccaagctcggaagatgacttttatgttgaagtggagagaaggtctgtgcaaaaaagtgatcagcagaatattaaagttccaggcaacttgacttcggtgctacttaacaacttacatcccagggagcagtacgtggtccgagctagagtcaacaccaaggcccagggggaatggagtgaagatctcactgcttggacccttagtgacattcttcctcctcaaccagaaaacatcaagatttccaacattacacactcctcagctgtgatttcttggacaatattggatggctattctatttcttctattactatccgttacaaggttcaaggcaagaatgaagaccagcacgttgatgtgaagataaagaatgccaccatcactcagtatcagctcaagggcctagagcctgaaacagcataccaggtggacatttttgcagagaacaacatagggtcaagcaacccagccttttctcatgaactggtgaccctcccagaatctcaagcaccagcggacctcggaggggggaagatgctgcttatagccatccttggctctgctggaatgacctgcctgactgtgctgttggcctttctgatcatattgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttacggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttccttcgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtatgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataccacgctttatgagaagtttacttatgcaggaattgactgttctgctgaagaagcggcctaggacagaacatctgtataccctctgtttccctttcactggcatgggagacccttgacacctgctgagaaaacatgcctctgccaaaggatgtgatatataagtgtacatatgtgctgtacacctgggaccttcaccactgtagatcccatgcatggatctatgtagtatgctctgactctaataggactgtatatactgttttaagaatgggctgaaatcagaatgcctgtttgtggtttcatatgcaataatatatttttttaaaaatgtggacttcataggaaggcgtgagtacaattagtataatgcataactcattgttgtcctagatattttgatatttacctttatgttgaatgctattaaatgttttcctgtgtcaaagtaaaatattgttaataaacctaacaatgaccctgatagtacaggttaagtgagagaactatatgaattctaacaagtcataggttaatatttaagacactgaaaaatctaagtgatataaatcagattcttctctctcaattttatccctcacctgtagcagccagtcccgtttcatttagtcatgtgaccactctgtcttgtgtttccacagcctgcaagtcagtccaggatgctaacatctaaaaatagacttaaatctcattgcttacaagcctaagaatctttagagaagtatacataagtttaggataaaataatgggattttcttttcttttctctggtaatattgacttgtatattttaagaaataacagaaagcctgggtgacatttgggagacatgtgacatttatatattgaattaatatccctacatgtattgcacattgtaaaaagttttagttttgatgagttgtgagtttaccttgtatactgtaggcacactttgcactgatatatcatgagtgaataaatgtcttgcctactcacgtctcaaaaaaaaaaaaaaaaaaaaaaaaaaaaaaaaaaaaaaaaaaaaaaaaaaaaaaaaaaa

SEQ ID NO: 650 - DNA, 4654 bp, Mus musculus
gagcaggagccggagcaggagcagaagataagccttggatgaagggcaagatggatagggctcgctctgccccaagccctgctgataccaagtgcctttaagatacagcctttcccatcctaatctgcaaaggaaacaggaaaaaggaacttaaccctccctgtgctcagacagaaatgagactgttaccgcctgcttctgtggtgtttctccttgccgccaacttgtaaacaagagcgagtggaccatgcgagcgggaagtcgcaaagttgtgagttgttgaaagcttcccagggactcatgctcatctgtggacgctggatggggagatctggggaagtatggactctttagccggcttagttctctgtggagtcagcttgctcctttatggagtagtagaaggcgccatggacctgatcttgatcaattccctacctcttgtgtctgatgccgaaacatccctcacctgcattgcctctgggtggcacccccatgagcccatcaccataggaagggactttgaagccttaatgaaccagcaccaagatccactggaggttactcaagatgtgaccagagaatgggcgaaaaaagttgtttggaagagagaaaaggccagtaagattaatggtgcttatttctgtgaaggtcgagttcgaggacaggctataaggatacggaccatgaagatgcgtcaacaagcatccttcctacctgctactttaactatgaccgtggacaggggagataatgtgaacatatctttcaaaaaggtgttaattaaagaagaagatgcagtgatttacaaaaatggctccttcatccactcagtgccccggcatgaagtacctgatattttagaagttcacttgccgcatgctcagccccaggatgctggtgtgtactcggccaggtacataggaggaaacctgttcacctcagccttcaccaggctgattgttcggagatgtgaagctcagaagtgggggcccgactgtagccgtccttgtactacttgcaagaacaatggagtctgccatgaagataccggggaatgcatttgccctcctgggtttatggggagaacatgtgagaaagcttgtgagccgcacacatttggcaggacctgtaaagaaaggtgtagtggaccagaaggatgcaagtcttatgtgttctgtctcccagacccttacgggtgttcctgtgccacaggctggagggggttgcagtgcaatgaagcatgcccatctggttactacggaccagactgtaagctcaggtgccactgtaccaatgaagagatatgtgatcggttccaaggatgcctctgctctcaaggatggcaagggctgcagtgtgagaaagaaggcaggccaaggatgactccacagatagaggatttgccagatcacattgaagtaaacagtggaaaatttaaccccatctgcaaagcctctgggtggccactacctactagtgaagaaatgaccctagtgaagccagatgggacagtgctccaaccaaatgacttcaactatacagatcgtttctcagtggccatattcactgtcaaccgagtcttacctcctgactcaggagtctgggtctgcagtgtgaacacagtggctgggatggtggaaaagcctttcaacatttccgtcaaagttcttccagagcccctgcacgccccaaatgtgattgacactggacataactttgctatcatcaatatcagctctgagccttactttggggatggacccatcaaatccaagaagcttttctataaacctgtcaatcaggcctggaaatacattgaagtgacgaatgagattttcactctcaactacttggagccgcggactgactacgagctgtgtgtgcagctggcccgtcctggagagggtggagaagggcatcctgggcctgtgagacgatttacaacagcgtctatcggactccctcctccaagaggtctcagtctcctgccaaaaagccagacagctctaaatttgacttggcaaccgatatttacaaactcagaagatgaattttatgtggaagtcgagaggcgatccctgcaaacaacaagtgatcagcagaacatcaaagtgcctgggaacctgacctcggtgctactgagcaacttagtccccagggagcagtacacagtccgagctagagtcaacaccaaggcgcagggggagtggagtgaagaactcagggcctggacccttagtgacattctccctcctcaaccagaaaacatcaagatctccaacatcactgactccacagctatggtttcttggacaatagtggatggctattcgatttcttccatcatcatccggtataaggttcagggcaaaaatgaagaccagcacattgatgtgaagatcaagaatgctaccgttactcagtaccagctcaagggcctagagccagagactacataccatgtggatatttttgctgagaacaacataggatcaagcaacccagccttttctcatgaactgaggacgcttccacattccccagcctctgcagacctcggagggggaaagatgctactcatagccatccttgggtcggctggaatgacttgcatcaccgtgctgttggcgtttctgattatgttgcaactgaagagagcaaatgtccaaaggagaatggctcaggcattccagaacgtgagagaagaaccagctgtgcagtttaactcaggaactctggcccttaacaggaaggccaaaaacaatccggatcccacaatttatcctgtgcttgactggaatgacatcaagtttcaagacgtgatcggagagggcaactttggccaggttctgaaggcacgcatcaagaaggatgggttacggatggatgccgccatcaagaggatgaaagagtatgcctccaaagatgatcacagggacttcgcaggagaactggaggttctttgtaaacttggacaccatccaaacatcattaatctcttgggagcatgtgaacaccgaggctatttgtacctagctattgagtatgccccgcatggaaacctcctggacttcctgcgtaagagcagagtgctagagacagaccctgcttttgccatcgccaacagtacagcttccacactgtcctcccaacagcttcttcattttgctgcagatgtggcccgggggatggactacttgagccagaaacagtttatccacagggacctggctgccagaaacattttagttggtgaaaactacatagccaaaatagcagattttggattgtcacgaggtcaagaagtgtatgtgaaaaagacaatgggaaggctcccagtgcgttggatggcaatcgaatcactgaactatagtgtctatacaaccaacagtgatgtctggtcctatggtgtattgctctgggagattgttagcttaggaggcaccccctactgcggcatgacgtgcgcggagctctatgagaagctaccccagggctacaggctggagaagcccctgaactgtgatgatgaggtgtatgatctaatgagacagtgctggagggagaagccttatgagagaccatcatttgcccagatattggtgtccttaaacaggatgctggaagaacggaagacatacgtgaacaccacactgtatgagaagtttacctatgcaggaattgactgctctgcggaagaagcagcctagagcagaactcttcatgtacaacggccatttctcctcactggcgcgagagcgccttgacacctgtaccaagcaagccacccactgccaagagatgtgatatataagtgtatatattgtgctgtgtttgggaccctcctcatacagctcgtgcggatctgcagtgtgttctgactctaatgtgactgtatatactgctcggagtaagaatgtgctaagatcagaatgcctgttcgtggtttcatataatatatttttctaaaagcatagattgcacaggaaggtatgagtacaaatactgtaatgcataacttgttattgtcctagatgtgtttgatatttttcctttacaactgaatgctataaaagtgttttgctgtgtacacataagatactgttcgttaaaataagcattcccttgacagcacaggaagaaaagcgagggaaatgtatggattatattaaatgtgggttactacacaagaggccgaacattccaagtagcagaagagagggtctctcaactctgctcctcacctgcagaagccagtttgtttggccatgtgacaattgtcctgtgtttttatagcacccaaatcattctaaaatatgaacatctaaaaactttgctaggagactaagaacctttggagagatagatataagtacggtcaaaaaacaaaactgtgggacttacatttattttctatagtaatctgttgtacattttaagaagtaaaactaggaatttaggagtgatgtgtgacatttctgacatggagttaccatccccacatgtatcacatactgtcatattcccacatgtatcacacatgtattgtaaaattttgtagttttgatcacttgtgaatttactgttgatgtggtagccacctgctgcaatggttcctcttgtaggtgaataaatgtcttgtctacccaca

SEQ ID NO: 651 - DNA, 4338 bp, Homo sapiens
ggggcacactcatgcattcctgtcaagtcatcttgtgaaaggctgcctgcttccagcttggcttggatgtgcaaccttaataaaactcactgaggtctgggagaaaatagcagatctgcagcagatagggtagaggaaagggtctagaatatgtacacgcagctgactcaggcaggctccatgctgaacggtcacacagagaggaaacaataaatctcagctactatgcaataaatatctcaagttttaacgaagaaaaacatcattgcagtgaaataaaaaattttaaaattttagaacaaagctaacaaatggctagttttctatgattcttcttcaaacgctttctttgagggggaaagagtcaaacaaacaagcagttttacctgaaataaagaactagttttagaggtcagaagaaaggagcaagttttgcgagaggcacggaaggagtgtgctggcagtacaatgacagttttcctttcctttgctttcctcgctgccattctgactcacatagggtgcagcaatcagcgccgaagtccagaaaacagtgggagaagatataaccggattcaacatgggcaatgtgcctacactttcattcttccagaacacgatggcaactgtcgtgagagtacgacagaccagtacaacacaaacgctctgcagagagatgctccacacgtggaaccggatttctcttcccagaaacttcaacatctggaacatgtgatggaaaattatactcagtggctgcaaaaacttgagaattacattgtggaaaacatgaagtcggagatggcccagatacagcagaatgcagttcagaaccacacggctaccatgctggagataggaaccagcctcctctctcagactgcagagcagaccagaaagctgacagatgttgagacccaggtactaaatcaaacttctcgacttgagatacagctgctggagaattcattatccacctacaagctagagaagcaacttcttcaacagacaaatgaaatcttgaagatccatgaaaaaaacagtttattagaacataaaatcttagaaatggaaggaaaacacaaggaagagttggacaccttaaaggaagagaaagagaaccttcaaggcttggttactcgtcaaacatatataatccaggagctggaaaagcaattaaacagagctaccaccaacaacagtgtccttcagaagcagcaactggagctgatggacacagtccacaaccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttgaaagcgcaatgtcagaagcgattatgaaagcaacaaagaaatccggagaagctgccaggtgagaaactgtttgaaaacttcagaagcaaacaatattgtctcccttccagcaataagtggtagttatgtgaagtcaccaaggttcttgaccgtgaatctggagccgtttgagttcacaagagtctctacttggggtgacagtgctcacgtggctcgactatagaaaactccactgactgtcgggctttaaaaagggaagaaactgctgagcttgctgtgcttcaaactactactggaccttattttggaactatggtagccagatgataaatatggttaatttcatgtaaaacagaaaaaaagagtgaaaaagagaatatacatgaagaatagaaacaagcctgccataatcctttggaaaagatgtattataccagtgaaaaggtgttatatctatgcaaacctactaacaaattatactgttgcacaattttgataaaaatttagaacagcattgtcctctgagttggttaaatgttaatggatttcagaagcctaattccagtatcatacttactagttgatttctgcttacccatcttcaaatgaaaattccatttttgtaagccataatgaactgtagtacatggacaataagtgtgtggtagaaacaaactccattactctgatttttgatacagttttcagaaaaagaaatgaacataatcaagtaaggatgtatgtggtgaaaacttaccacccccatactatggttttcatttactctaaaaactgattgaatgatatataaatatatttatagcctgagtaaagttaaaagaatgtaaaatatatcatcaagttcttaaaataatatacatgcatttaatatttcctttgatattatacaggaaagcaatattttggagtatgttaagttgaagtaaaagcaagtactctggagcagttcattttacagtatctacttgcatgtgtatacatacatgtaacttcattattttaaaaatatttttagaactccaatactcaccctgttatgtcttgctaatttaaattttgctaattaactgaaacatgcttaccagattcacactgttccagtgtctataaaagaaacactttgaagtctataaaaaataaaataattataaatatcattgtacatagcatgtttatatctgcaaaaaacctaatagctaattaatctggaatatgcaacattgtccttaattgatgcaaataacacaaatgctcaaagaaatctactatatcccttaatgaaatacatcattcttcatatatttctccttcagtccattcccttaggcaatttttaatttttaaaaattattatcaggggagaaaaattggcaaaactattatatgtaagggaaatatatacaaaaagaaaattaatcatagtcacctgactaagaaattctgactgctagttgccataaataactcaatggaaatattcctatgggataatgtattttaagtgaatttttggggtgcttgaagttactgcattattttatcaagaagtcttctctgcctgtaagtgtccaaggttatgacagtaaacagtttttattaaaacatgagtcactatgggatgagaaaattgaaataaagctactgggcctcctctcataaaagagacagttgttggcaaggtagcaataccagtttcaaacttggtgacttgatccactatgccttaatggtttcctccatttgagaaaataaagctattcacattgttaagaaaaatactttttaaagtttaccatcaagtcttttttatatttatgtgtctgtattctacccctttttgccttacaagtgatatttgcaggtattataccatttttctattcttggtggcttcttcatagcaggtaagcctctccttctaaaaacttctcaactgttttcatttaagggaaagaaaatgagtattttgtccttttgtgttcctacagacactttcttaaaccagtttttggataaagaatactatttccaaactcatattacaaaaacaaaataaaataataaaaaaagaaagcatgatatttactgttttgttgtctgggtttgagaaatgaaatattgtttccaattatttataataaatcagtataaaatgttttatgattgttatgtgtattatgtaatacgtacatgtttatggcaatttaacatgtgtattcttttaattgtttcagaataggataattaggtattcgaattttgtctttaaaattcatgtggtttctatgcaaagttcttcatatcatcacaacattatttgatttaaataaaattgaaagtaatatttgtgcaa

SEQ ID NO: 652 - DNA, 4078 bp, Mus musculus
ggaaaggggctagaatatgtactcgcagctgacgcgggcaggctccacgctgaacggttacacagagaggaaacaataaatctaagctactattgcaataaatatctcaagttttaacgaaggaaactatcattacagttaaaattttttaaagtaacgcttttttagaacaaagctaacaaatggctagttttctgtggatcttcttcaaacgctttctttaacggggaaagagtcaaacaagcagttttacctgaaataaagaactagtttaaaggtcagaagagaagagcaagctttgcaggaggcacggaaggcaagcgctggcagtacaatgacagttttcctttcctttgcattcttcgctgccattctgactcacatagggtgcagcaaccagcgccgaaatccagaaaacggagggagaagatataaccggattcaacatgggcaatgtgcctacactttcattcttccagaacacgacgggaactgccgtgagagtgcgacagagcagtacaacaccaacgctctgcaaagggatgctccacacgtggagccggatttctcttcccagaaacttcagcatctggagcatgtgatggaaaattatactcagtggctgcaaaaacttgagaattacattgtggaaaatatgaagtcggagatggcccagatacaacagaatgctgttcaaaaccacacggccaccatgcttgagataggaaccagtctcttatctcagactgcagagcagacccgaaagctgacagatgttgagacccaggtactaaatcaaacatcccgacttgaaatacaactgctagagaattcattatcaacatacaagctagagaagcaacttctccaacagacaaatgaaattctgaagattcacgaaaaaaacagtttactagagcacaaaatcttagaaatggagggaaaacacaaagaagaattggacaccttgaaggaggagaaagaaaaccttcaaggcttggtttctcgtcagacattcatcatccaggagttggagaagcaacttagtagagctaccaacaacaacagcatcctgcagaagcaacaactggagctcatggacacagttcataaccttatcagcctttgcactaaagaaggtgttttgctaaagggaggaaaaagagaagaagagaaaccatttcgagactgtgcagatgtatatcaagctggttttaataaaagtggaatctacactatttattttaataatatgccagaacccaaaaaggtattttgcaatatggatgtgaatgggggaggttggacagtaatacaacaccgggaagatggaagcctggatttccagaggggctggaaggagtataaaatgggttttgggaatccctctggtgaatattggcttgggaacgagttcatttttgcaataaccagtcagaggcagtacatgctgaggattgagctgatggactgggaagggaaccgagcctactcacagtacgacagattccacataggaaatgaaaagcagaactataggttatatttaaaaggtcacacagggacagcaggcaaacagagcagcttgatcttacacggtgccgatttcagcacgaaggatgctgataacgacaactgtatgtgcaaatgcgctctcatgctaacaggaggttggtggttcgatgcctgtggcccttccaatctaaatggaatgttctacactgcgggacaaaatcatggaaaactgaatgggataaagtggcactacttcaaagggcccagttactccttacgttccaccaccatgatgatccggcccttggacttttgaaggtgctctgccagtattagaaagctgcaaagaaagctgggcatgttcccagatgagaagctagtcagaggcttcagaaacaaccaacattgtctccattccagcagcaagtggttatgtcatgtcacctgggtttggagccttctgaggtcaacagaatcgccacttgggtccagagaatgccactcacaatcatgtttaaaagggaagaaacttctcagcttgctgcacttcaaagtgctactggatcacattctgaacttataacatcctgatgctgaatgcaacttgtttcatgtaaaagcaaaagaagaagaaacagcaaatgggaacaggctttccagaatctgttgaagatggattgtggaggtgacctggtatcactgtaggaaatcctgctaacaatacatcactgcccaaaagagacataaagaaaagttttgtctactgagttggctaaaagttagtggagttcacctgcccatttccagtatcatatttactagctgatttcaggtttcctgtgttcaaatgtaaactctgttcttgtaagccatgatacaatatagtacatggaggataagagttgggggtagaaggtgcctaaagactcttgagtttctggggattcagttttcaaaagatataaaatataatcaagaatggataaaacaggtgaaaatcacactcatgctacagtgttcctttacatgaaatttgattaactgatccacaagaatgtttagagcctgagtatatataaagactggaagtgttatcacccagttctcaaaacaataagcaggcagttaacattctcattgacagtatgtaggagagcaatatgtggagtacttgagttggaacagcccattgtacagatcttgcatgtatttgcatatgtatggcattattatttttaaagtgttcgtaggccttcaattcttcatacagatttttcatgctaatttaatttttgttaattaactgcaatgtacttactaaatatatcctactccagttttttatgagttatactttaaagtctacaaataatagaagaattttaaatatcattgtacataatatcttatacctgtccatgctaaactcaataattgtttagtctggaatatatgatgctgtccacaactgatgactataaatatgattgtttaaagacagttaccatactattgattaaatatattactctgcatagtttttctcctccaggatctgtttcttcaagcaatttctaccttgtaaaataatggtagtagagaaaattgacataactccttgtacaaaagaattatagaaaaaattacagtcatttgactaggaagtttctgattgttagctgctataagtgccttagttaagatgcccctgtgttataatatgtagtaaatgaagttttggacacaggattctgtgataacctgatgtgactgcagtattctatcaagttctctttgttgttaaatgttcaaggttatagtagaaaaaaaacattcaatcaaacacaatttgccatgaaaggagagaactaaatgtaggcaccagttctgttttctcagagaaggagaagactttctgggacgtacatgtaccaaaatataaatcttgataaccgcagccacaaagccttagtgactttcctctacctggtaagacagagctcttcatgcttttaagaaaagattctgaatgcttcccaccacatctttcttatatttatatgtgttcataaagtactattttgccttacaagaggtatgtgccgacattacaggatttttctactatagtgactccttcacagctttcttaagcctagccctctaaaagcttccttctcatttagatgaaagaaaatgagtatttttgtgattctggtgattgtggtggttgttgttgttgttgttgttgttcccacagatgttcgaaaactcatcttgggtaaattgtttttcaatccacattacaaaaataaagcgaaacaaggagaaaaaaaagcatggaatttactgatttgttatgtgggtttgaaaaataagatattgttttcagttatttataataaagcagtataatgtgtacattgtataatgccaacatgtgtgtagcaatttgatacgcatagctttttgcatttaattaatgcagggcagaaaaattagataactcgaactttgtcttgaagtttctatttcaataaaagctgtgtcatttctatgaaaa

SEQ ID NO: 653 - DNA, 5270 bp, Homo sapiens
aaagtgattgattcggatactgacactgtaggatctggggagagaggaacaaaggaccgtgaaagctgctctgtaaaagctgacacagccctcccaagtgagcaggactgttcttcccactgcaatctgacagtttactgcatgcctggagagaacacagcagtaaaaaccaggtttgctactggaaaaagaggaaagagaagactttcattgacggacccagccatggcagcgtagcagccctgcgttttagacggcagcagctcgggactctggacgtgtgtttgccctcaagtttgctaagctgctggtttattactgaagaaagaatgtggcagattgttttctttactctgagctgtgatcttgtcttggccgcagcctataacaactttcggaagagcatggacagcataggaaagaagcaatatcaggtccagcatgggtcctgcagctacactttcctcctgccagagatggacaactgccgctcttcctccagcccctacgtgtccaatgctgtgcagagggacgcgccgctcgaatacgatgactcggtgcagaggctgcaagtgctggagaacatcatggaaaacaacactcagtggctaatgaagcttgagaattatatccaggacaacatgaagaaagaaatggtagagatacagcagaatgcagtacagaaccagacggctgtgatgatagaaatagggacaaacctgttgaaccaaacagcggagcaaacgcggaagttaactgatgtggaagcccaagtattaaatcagaccacgagacttgaacttcagctcttggaacactccctctcgacaaacaaattggaaaaacagattttggaccagaccagtgaaataaacaaattgcaagataagaacagtttcctagaaaagaaggtgctagctatggaagacaagcacatcatccaactacagtcaataaaagaagagaaagatcagctacaggtgttagtatccaagcaaaattccatcattgaagaactagaaaaaaaaatagtgactgccacggtgaataattcagttcttcagaagcagcaacatgatctcatggagacagttaataacttactgactatgatgtccacatcaaactcagctaaggaccccactgttgctaaagaagaacaaatcagcttcagagactgtgctgaagtattcaaatcaggacacaccacgaatggcatctacacgttaacattccctaattctacagaagagatcaaggcctactgtgacatggaagctggaggaggcgggtggacaattattcagcgacgtgaggatggcagcgttgattttcagaggacttggaaagaatataaagtgggatttggtaacccttcaggagaatattggctgggaaatgagtttgtttcgcaactgactaatcagcaacgctatgtgcttaaaatacaccttaaagactgggaagggaatgaggcttactcattgtatgaacatttctatctctcaagtgaagaactcaattataggattcaccttaaaggacttacagggacagccggcaaaataagcagcatcagccaaccaggaaatgattttagcacaaaggatggagacaacgacaaatgtatttgcaaatgttcacaaatgctaacaggaggctggtggtttgatgcatgtggtccttccaacttgaacggaatgtactatccacagaggcagaacacaaataagttcaacggcattaaatggtactactggaaaggctcaggctattcgctcaaggccacaaccatgatgatccgaccagcagatttctaaacatcccagtccacctgaggaactgtctcgaactattttcaaagacttaagcccagtgcactgaaagtcacggctgcgcactgtgtcctcttccaccacagagggcgtgtgctcggtgctgacgggacccacatgctccagattagagcctgtaaactttatcacttaaacttgcatcacttaacggaccaaagcaagaccctaaacatccataattgtgattagacagaacacctatgcaaagatgaacccgaggctgagaatcagactgacagtttacagacgctgctgtcacaaccaagaatgttatgtgcaagtttatcagtaaataactggaaaacagaacacttatgttatacaatacagatcatcttggaactgcattcttctgagcactgtttatacactgtgtaaatacccatatgtcctgaattcaccatcactatcacaattaaaaggaagaaaaaaactctctaagccataaaaagacatattcagggatattctgagaaggggttactagaagtttaatatttggaaaaacagttagtgcatttttactccatctcttaggtgctttaaatttttatttcaaaaacagcgtatttacatttatgttgacagcttagttataagttaatgctcaaatacgtatttcaaatttatatggtagaaacttccagaatctctgaaattatcaacagaaacgtgccattttagtttatatgcagaccgtactatttttttctgcctgattgttaaatatgaaggtatttttagtaattaaatataacttattaggggatatgcctatgtttaacttttatgataatatttacaattttataatttgtttccaaaagacctaattgtgccttgtgataaggaaacttcttacttttaatgatgaggaaaattatacatttcattctatgacaaagaaactttactatcttctcactattctaaaacagaggtctgttttctttcctagtaagatatatttttatagaactagactacaatttaatttctggttgagaaaagccttctatttaagaaatttacaaagctatatgtctcaagattcacccttaaatttacttaaggaaaaaaataattgacactagtaagtttttttatgtcaatcagcaaactgaaaaaaaaaaaagggtttcaaagtgcaaaaacaaaatctgatgttcataatatatttaaatatttaccaaaaatttgagaacacagggctgggcgcagtggctcacacctataatcccagtacattggtaggcaaggtgggcagatcacctgaggtcaggagttcaagaccagcctggacaacatggtgaaaccctgtctctactaaataatacaaaaattagccaggcgtgctggcgggcacctgtaatcccagctactcgggaggctgaggcagggagaattgcttgcaccagggaggtagaggttgcagtgagccaagatcgcaccactgcactccagccggggcaacagagcaagactccatctcaaaaaaaaaaaaaaaaaaagaaagaaaagaaaatttgagaacacagctttatactcgggactacaaaaccataaactcctggagttttaactccttttgaaattttcatagtacaattaatactaatgaacatttgtgtaaagctttataatttaaaggcaatttctcatatattcttttctgaatcatttgcaaggaagttcagagtccagtctgtaactagcatctactatatgtctgtcttcaccttacagtgttctaccattattttttctttattccatttcaaaatctaatttattttaccccaacttctccccaccacttgacgtagttttagaacacacaggtgttgctacatatttggagtcaatgatggactctggcaaagtcaaggctctgttttatttccaccaaggtgcacttttccaacaactatttaactagttaagaacctccctatcttagaactgtatctactttatatttaagaaggttttatgaattcaacaacggtatcatggccttgtatcaagttgaaaaacaactgaaaataagaaaatttcacagcctcgaaagacaacaacaagtttctaggatatctcaatgacaagagtgatggatacttaggtagggaaacgctaatgcaggaaaaactggcaacaacacaatttatatcaattctctttgtaggcaggtgataaaaaattcaaggacaaatctcattatgtcattgtgcatcatatataatctcttatgagcgagaatggggggaatttgtgtttttactttacacttcaattccttacacggtatttcaaacaaacagttttgctgagaggagcttttgtctctccttaagaaaatgtttataaagctgaaaggaaatcaaacagtaatcttaaaaatgaaaacaaaacaacccaacaacctagataactacagtgatcagggagcacagttcaactccttgttatgttttagtcatatggcctactcaaacagctaaataacaacaccagtggcagataaaaatcaccatttatctttcagctattaatcttttgaatgaataaactgtgacaaacaaattaacatttttgaacatgaaaggcaacttctgcacaatcctgtatccaagcaaactttaaattatccacttaattattacttaatcttaaaaaaaattagaacccagaacttttcaatgaagcatttgaaagttgaagtggaatttaggaaagccataaaaatataaatactgttatcacagcaccagcaagccataatctttatacctatcagttctatttctattaacagtaaaaacattaagcaagatataagactacctgcccaagaattcagtcttttttcatttttgtttttctcagttctgaggatgttaatcgtcaaattttctttggactgcattcctcactactttttgcacaatggtctcacgttctcacatttgttctcgcgaataaattgataaaaggtgttaagttctgtgaatgtctttttaattatgggcataattgtgcttgactggataaaaacttaagtccacccttatgtttataataatttcttgagaacagcaaactgcatttaccatcgtaaaacaacatctgacttacgggagctgcagggaagtggtgagacagttcgaacggctcctcagaaatccagtgacccaattctaaagaccatagcacctgcaagtgacacaacaagcagatttattatacatttattagccttagcaggcaataaaccaagaatcactttgaagacacagcaaaaagtgatacactccgcagatctgaaatagatgtgttctcagacaacaaagtcccttcagaatcttcatgttgcataaatgttatgaatattaataaaaagttgattgagaaaaa

SEQ ID NO: 654 - DNA, 2475 bp, Mus musculus
gatactgacactgtagactcaggggagaaacaaagagtccgtgcagacctctggagtgagcagggctgctccttcctctcaggacagctccgagtgtgccggggagaagagaagagaagagacaggcactgggaaagagcctgctgcgggacggagaaggctctcactgatggacttattcacacggcacagccctgtgccttagacagcagctgagagctcaggacgcaagtttgctgaactcacagtttagaacccaaaaagagagagagaatgtggcagatcattttcctaacttttggctgggatcttgtcttggcctcagcctacagtaactttaggaagagcgtggacagcacaggcagaaggcagtaccaggtccagaacggaccctgcagctacacgttcctgctgccggagaccgacagctgccgatcttcctccagcccctacatgtccaatgccgtgcagagggatgcacccctcgactacgacgactcagtgcaaaggctgcaggtgctggagaacattctagagaacaacacacagtggctgatgaagctggagaattacattcaggacaacatgaagaaggagatggtggagatccaacagaatgtggtgcagaaccagacagctgtgatgatagagattggaaccagcttgctgaaccagacagcagcacaaactcggaaactgactgatgtggaagcccaagtactaaaccagacgacaagactcgagctgcagcttctccaacattctatttctaccaacaaattggaaaagcagattttggatcagaccagtgaaataaacaagctacaaaataagaacagcttcctagaacagaaagttctggacatggagggcaagcacagcgagcagctacagtccatgaaggagcagaaggacgagctccaggtgctggtgtccaagcagagctctgtcattgacgagctggagaagaagctggtgacagccacggtcaacaactcgctccttcagaagcagcagcatgacctaatggagaccgtcaacagcttgctgaccatgatgtcatcacccaactccaagagctcggttgctatccgtaaagaagagcaaaccaccttcagagactgtgcggaaatcttcaagtcaggactcaccaccagtggcatctacacactgaccttccccaactccacagaggagatcaaggcctactgtgacatggacgtgggtggaggagggtggacagtcatccaacaccgagaagatggcagtgtggacttccagaggacgtggaaagaatacaaagagggcttcgggagccctctgggagagtactggctgggcaatgagtttgtctcccagctgaccggtcagcaccgctacgtgcttaagatccagctgaaggactgggaaggcaacgaggcgcattcgctgtatgatcacttctacctcgctggtgaagagtccaactacaggattcaccttacaggactcacggggaccgcgggcaaaataagtagcatcagccaaccaggaagtgattttagcacaaaggattcggacaatgacaaatgcatctgcaagtgttcccagatgctctcaggaggctggtggtttgacgcatgtggtccttccaacttgaatggacagtactacccacaaaaacagaatacaaataagtttaacggtatcaagtggtactactggaaggggtccggctactcgctcaaggccacaaccatgatgatccggccagcagatttctaaatgcctgcctacactaccagaagaacttgctgcatccaaagattaactccaaggcactgagagacaccaatgcatagcagcccctttccacatcaggaagtgctcctgggggtggggagggtctgtgtgtaccagactgaagcgcatcacttaagcctgcaccgctaaccaaccaaaggcactgcagtctggagaaacacttctgggaaggttgtggctgaggatcagaaggacagcgtgcagactctgtcacagggaagaatgttccgtgggagttcagcagtaaataactggaaaacagaacacttagatggtgcagataaatcttgggaccacattcctctaagcacggtttctagagtgaatacattcacagctcggctgtcacaatgacaaggccgtgtcctcgcactgtggcagccagtatccagggacttctaagtggtgggcacaggttatcatctggagaagcacacattcattgttttcctcttgggtgctttacatgttcatttgaaaacaacacatttacctatcttgatggcttagtttttaatggctggctactatttactatatggcaaaaatgcccacatctctggaataaccaccaaataagcgccatgttggtgaatgcggagactgtactattttgttttcttcctggctgttaaatatgaaggtatttttagtaattaaatataagttatt

SEQ ID NO: 655 - protein, 4 aa, Artificial Sequence
  Description of Artificial Sequence: Synthetic peptide
KHHH

SEQ ID NO: 656 - protein, 75 aa, Artificial Sequence
  Description of Artificial Sequence: Synthetic peptide
KHHHKHHHKHHHHKHHHKKHHHKHHHKHHHHKHHHKKHHHKHHHKHHHHKHHHKKHHHKHHHKHHHHKHHHKKKK

SEQ ID NO: 657 - protein, 79 aa, Artificial Sequence
  Description of Artificial Sequence: Synthetic peptide
CKHHHKHHHKHHHHKHHHKCKHHHKHHHKHHHHKHHHKCKHHHKHHHKHHHHKHHHKCKHHHKHHHKHHHHKHHHKKKK

SEQ ID NO: 658 - protein, 5 aa, Artificial Sequence
  Description of Artificial Sequence: Synthetic peptide
RGDFK

SEQ ID NO: 659 - protein, 6 aa, Artificial Sequence
  Description of Artificial Sequence: Synthetic peptide
RGDDFK

(19) United States
(12) Patent Application Publication
(10) Pub. No.: US 2011/0015249 A1
(43) Pub. Date: Jan. 20, 2011

(54) METHODS AND COMPOSITIONS FOR TREATMENT OF CANCER AND OTHER ANGIOGENESIS-RELATED DISEASES

(75) Inventors: Frank Y. Xie, Germantown, MD (US); Xiaodong Yang, Palo Alto, CA (US); Yijia Liu, Gaithersburg, MD (US); Qing Zhou, Fremont, CA (US)

Correspondence Address: ROPES & GRAY LLP, PATENT DOCKETING 39/361, 1211 AVENUE OF THE AMERICAS, NEW YORK, NY, 10036-8704, US

(73) Assignee: INTRADIGM CORPORATION

(21) Appl. No.: 12/667,889

(22) PCT Filed: Jul. 3, 2008
(86) PCT No.: PCT/US08/08232

§ 371 Date: Jan. 6, 2010

Related U.S. Application Data
(60) Provisional application No. 60/958,519, filed on Jul. 6, 2007.
(60) Provisional application No. 60/966,085, filed on Aug. 24, 2007.
(60) Provisional application No. 61/131,876, filed on Jun. 12, 2008.

(51) Int. Cl. A61K 31/7088; C07H 21/02; C12N 5/02; A61P 35/00

(52) U.S. Cl. 514/44 A; 536/24.5; 435/375; 977/773; 977/904

(57) ABSTRACT

The present invention provides nucleic acid molecules that modulate the expression of molecules in the angiopoietin/Tie2 signaling pathway. Methods of using the nucleic acid molecules are also provided.

### CROSS-REFERENCE TO RELATED APPLICATIONS

This application claims priority under 35 U.S.C. §119(e) from U.S. provisional application 60/958,519, filed Jul. 6, 2007, U.S. provisional application 60/966,085, filed Aug. 24, 2007 and U.S. provisional application 61/131,876, filed Jun. 12, 2008.

### FIELD OF THE INVENTION

The present invention is in the field of molecular biology and medicine and relates to short interfering RNA (siRNA) molecules for modulating the expression of molecules in the angiopoietin/Tie2 signaling pathway.

### BACKGROUND OF THE INVENTION

The angiopoietin/Tie2 signaling pathway has been implicated in several types of cancer-induced angiogenesis. Several molecules in the Ang-Tie pathway have been identified (see, e.g., Tables 1 and 13). One of them is the receptor molecule Tie2 (Tyrosine Kinase with Immunoglobulin and EGF factor homology domains, also called TIE-2, TEK or epithelial-specific protein receptor tyrosine kinase, TEK tyrosine kinase), which is expressed almost exclusively on the surface of vascular endothelial cells (ECs) (Sato et al., 1998, Int. Immunol. 10: 1217-1227). Ligands that bind to Tie2 include angiopoietin-1 and angiopoietin-2 (Yancopoulos et al., 2000, Nature 407: 242-248).

TABLE 1
Angiopoietin/Tie2 pathway gene sequence IDs.

| UniGene | | Gene |
|---|---|---|
| Sequence ID | Gene Name | Abbreviation |
| Hs.89640 | H. sapiens receptor protein- | Hu Tie2 |
| | tyrosine kinase | |
| Mm.14313 | M. musculus Tie2 | Ms Tie2 |
| Hs.369675 | H. sapiens angiopoietin 1 | Hu Ang-1 |
| Mm.309336 | M. musculus angiopoietin 1 | Ms Ang-1 |
| Hs.583870 | H. sapiens angiopoietin 2 | Hu Ang-2 |
| Mm.435498 | M. musculus angiopoietin 2 | Ms Ang-2 |

Accordingly, there is an urgent need for therapeutic agents targeting the Ang-Tie pathway.

### SUMMARY OF THE INVENTION

One aspect of the present invention provides a nucleic acid molecule that reduces expression of an angiopoietin-1 (Ang-1), an angiopoietin-2 (Ang-2), or a tyrosine kinase with immunoglobulin and EGF factor homology domains (Tie2) gene, wherein the nucleic acid molecule comprises or targets any one of SEQ ID NOs: 1-648. The present invention also provides a nucleic acid molecule that reduces expression of an Ang-2 gene, wherein the nucleic acid molecule comprises or targets any one of SEQ ID NOs: 487, 489, 525, 526, 553, 554, 639, 640, 643, and 644. In a particular embodiment, the nucleic acid molecule is a short interfering RNA (siRNA) molecule. In a preferred embodiment, the invention provides siRNA of 25 base pairs with blunt ends.

The present invention also provides a composition comprising a nucleic acid molecule that comprises or targets any one of SEQ ID NOs: 1-648 and a pharmaceutically acceptable carrier. In one embodiment, the composition further comprises a histidine-lysine copolymer. In a further embodiment, the composition further comprises a targeting moiety. The composition may also comprise one or more additional therapeutic agents.

The present invention also provides combinations of nucleic acid molecules that target multiple disease-causing genes or target different sequences in the same gene. In one aspect, the invention provides compositions comprising a nucleic acid molecule that comprises or targets any one of SEQ ID NOs: 1-648 and further comprising one or more additional nucleic acid molecules that induce RNA interference and decrease the expression of a gene of interest. In one embodiment, the one or more additional nucleic acid molecules decrease the expression of Ang-1, Ang-2, or Tie-2.

The present invention further provides methods for reducing protein level expression of Ang-1, Ang-2, or Tie-2 genes in a cell, comprising introducing into the cell any of the nucleic acid molecules or the siRNA molecules of the invention. The present invention also provides methods of reducing angiogenesis in a subject in need thereof, comprising administering to the subject any of the nucleic acid molecules, siRNA molecules, or compositions of the invention. Additionally, the present invention provides a method of treating cancer in a subject in need thereof, comprising administering to the subject any of the nucleic acid molecules, siRNA molecules, or compositions of the invention.

These and other aspects of the present invention will become apparent upon references to the following detailed description.

### BRIEF DESCRIPTION OF THE DRAWINGS

FIG. 1 is a bar graph depicting in vitro inhibition of human Ang-2 by siRNA molecules in human umbilical vein endothelial (HUVEC) cells at 24 hours post siRNA transfection.

Human Ang-2 gene silencing activity of human Ang-2-siRNA sequences listed in Table 11 was tested in HUVEC cells. Labels #1-#48 on the x-axis correspond to the siRNA sequences numbered 1-48 in Table 11. The HUVEC cells were transfected with the Ang-2-siRNAs using a reverse transfection based high-through-put (HTP) method with 10 nM of siRNA duplex. A luciferase specific 25-mer siRNA was used as the negative control (Luc). The effect of siRNA mediated Ang-2 knockdown was monitored by measuring the concentration of Ang-2 protein in the medium using a human Ang-2 ELISA kit (R&D). Significant inhibition of Ang-2 protein level expression in transfected HUVEC cells was observed at 24 hours post transfection with a majority of the 48 Ang-2 siRNA candidates tested.

FIG. 2 is a bar graph depicting in vitro inhibition of human Ang-2 by siRNA molecules in HUVEC cells at 48 hours post siRNA transfection.

Human Ang-2 gene silencing activity of human Ang-2-siRNA sequences listed in Table 11 was tested in HUVEC cells. Labels 1-48 on the x-axis correspond to the siRNA sequences numbered 1-48 in Table 11. The HUVEC cells were transfected with the Ang-2-siRNAs using a reverse transfection based high-through-put (HTP) method with 10 nM of siRNA duplex. A luciferase specific 25-mer siRNA was used as the negative control (Luc). The effect of siRNA mediated Ang-2 knockdown was monitored by measuring the concentration of Ang-2 protein in the medium using a human Ang-2 ELISA kit (R&D). At 48 hours post siRNA transfection, more than 50% of the transfected HUVEC cells express less than 20% of Ang-2 protein compared to the mock control.

FIG. 3 is a bar graph depicting the percentage of inhibition of human Ang-2 by siRNA molecules in HUVEC cells at 48 hours post siRNA transfection.

Human Ang-2 gene silencing activity of human Ang-2-siRNA sequences listed in Table 11 was tested in HUVEC cells. Labels 1-48 on the x-axis correspond to the siRNA sequences numbered 1-48 in Table 11. The HUVEC cells were transfected with the Ang-2-siRNAs using a reverse transfection based high-through-put (HTP) method with 10 nM of siRNA duplex. A luciferase specific 25-mer siRNA was used as the negative control. The effect of siRNA mediated Ang-2 knockdown was monitored by measuring the concentration of Ang-2 protein in the medium using a human Ang-2 ELISA kit (R&D). At 48 hours post transfection, the inhibition effects of Ang-2 siRNA on Ang-2 expression were more profound, with more than 50% of the Ang-2 siRNA candidates showing a greater than 80% knockdown of Ang-2 expression compared to the cells transfected with control Luc-siRNA.

FIG. 4 is a bar graph depicting the cell viability of HUVEC cells transfected with 10 nM human Ang-2 siRNA molecules at 48 hours post siRNA transfection.

The HUVEC cells were transfected with the Ang-2-siRNAs using a reverse transfection based high-through-put (HTP) method with 10 nM of siRNA duplex. Labels 2-48 on the x-axis correspond to the siRNA sequences numbered 2-48 in Table 11. A luciferase specific 25-mer siRNA was used as the negative control (Luc). The cell viability of the transfected cells was measured using a WST-1 assay kit (Roche). There was no significant cytotoxicity in the transfected HUVEC cells that associated with knockdown of Ang-2 expression.

FIG. 5 is a bar graph depicting in vitro inhibition of human Ang-2 by siRNA molecules at 2 nM in HUVEC cells at 48 hours post siRNA transfection.

Human Ang-2 gene silencing activity of human Ang-2-siRNA sequences listed in Table 11 was further confirmed in HUVEC cells. Labels on the x-axis correspond to the siRNA sequences numbers in Table 11. The HUVEC cells were transfected with the Ang-2-siRNAs using a reverse transfection based high-through-put (HTP) method with 2 nM of siRNA duplex. A control (Ctrl-) siRNA, which has a 19-nt double-stranded region with dTdT 3′-overhangs on both strands and does not has a significant homologous sequence with any known human gene, was used as the negative control. The effect of siRNA mediated Ang-2 knockdown was monitored by measuring the concentration of Ang-2 protein in the medium using a human Ang-2 ELISA kit (R&D). At 48 hours post siRNA transfection, most of the transfected HUVEC cells express less than 16% of Ang-2 protein compared to mock control.

FIG. 6 is a bar graph depicting the percentage of inhibition of human Ang-2 by siRNA molecules at 2 nM in HUVEC cells at 48 hours post siRNA transfection

The HUVEC cells were transfected with the Ang-2-siRNAs using a reverse transfection based high-through-put (HTP) method with 2 nM of siRNA duplex. A control (Ctrl-) siRNA was used as the negative control. The effect of siRNA mediated Ang-2 knockdown was monitored by measuring the concentration of Ang-2 protein in the medium using a human Ang-2 ELISA kit (R&D). At 48 hours post transfection, a majority of the Ang-2 siRNAs demonstrated a greater than 90% knockdown of Ang-2 expression.

FIG. 7 is a bar graph depicting the cell viability of HUVEC cells transfected with 2 nM human Ang-2 siRNA molecules at 48 hours post siRNA transfection.

The HUVEC cells were transfected with the Ang-2-siRNAs using a reverse transfection based high-through-put (HTP) method with 2 nM of siRNA duplex. Labels on the x-axis correspond to the siRNA sequence numbers in Table 11. A control (Ctrl-) siRNA, which has a 19-nt double-stranded region with dTdT 3′-overhangs on both strands and does not has a significant homologous sequence with any known human gene, was used as the negative control. The cell viability of the transfected cells was measured using a WST-1 assay kit (Roche). There was no significant cytotoxicity in the transfected HUVEC cells that associated with knockdown of Ang-2 expression.

FIG. 8 is a bar graph depicting in vitro inhibition of human Ang-2 by siRNA molecules at 0.2 nM in HUVEC cells at 48 hours post siRNA transfection.

Human Ang-2 gene silencing activity of the human Ang-2-siRNA sequences listed in Table 11 was further confirmed in HUVEC cells. The number labels on the x-axis correspond to the siRNA sequence numbers in Table 11. The HUVEC cells were transfected with the Ang-2-siRNAs using a reverse transfection based high-through-put (HTP) method with 0.2 nM of siRNA duplex. A control (Ctrl-) siRNA was used as the negative control. The effect of siRNA mediated Ang-2 knockdown was monitored by measuring the concentration of Ang-2 protein in the medium using a human Ang-2 ELISA kit (R&D). At 48 hours post siRNA transfection, some of the transfected HUVEC cells express less than 60% of Ang-2 protein compared to mock control. siRNA sequence numbers circled were used for further experiments whose results are shown in FIGS. 9 and 10.

FIG. 9A-C shows three line graphs depicting the determination of IC50 values of the selected Ang-2 siRNA in HUVEC cells at 48 hours post siRNA transfection.

HUVEC cells were transfected with 10 dilutions of each siRNA duplex (#10 (FIG. 9A), #14 (FIG. 9B), and #31 (FIG. 9C) in Table 11). The dilutions were 0.076 pM, 0.31 pM, 1.2 pM, 4.9 pM, 19.5 pM, 78.1 pM, 312.5 pM, 1.25 nM, 5 nM, and 20 nM. The effect of siRNA mediated Ang-2 knockdown was monitored by measuring the concentration of Ang-2 protein in the medium using a human Ang-2 ELISA kit (R&D). The cell viability of the transfected cells was measured using a WST-1 assay kit (Roche) for normalization of Ang-2 concentration. The IC50 value of each siRNA duplex in HUVEC cells at 48 hours post siRNA transfection was obtained using the GraphPad Prism program. The IC50 of Ang-2-25-10 was 0.363 nM, the IC50 of Ang-2-25-14 was 0.494 nM, and the IC50 of Ang-2-25-31 was 0.398 nM.

FIG. 10A-B shows two line graphs depicting the determination of IC50 values of the selected human/mouse Ang-2 siRNA in HUVEC cells at 48 hours post siRNA transfection.

HUVEC cells were transfected with 10 dilutions of each siRNA duplex (#25 (FIG. 10A) and #45 (FIG. 10B) in Table 11). The dilutions were 0.076 pM, 0.31 pM, 1.2 pM, 4.9 pM, 19.5 pM, 78.1 pM, 312.5 pM, 1.25 nM, 5 nM, and 20 nM. The effect of siRNA mediated Ang-2 knockdown was monitored by measuring the concentration of Ang-2 protein in the medium using a human Ang-2 ELISA kit (R&D). The cell viability of the transfected cells was measured using a WST-1 assay kit (Roche) for normalization of Ang-2 concentration. The IC50 value of each siRNA duplex in HUVEC cells at 48 hours post siRNA transfection was obtained using the GraphPad Prism program. The IC50 of Ang-2-25-25 was 1.634 nM, and the IC50 of Ang-2-25-45 was 0.90 nM.

### DETAILED DESCRIPTION OF THE INVENTION

The invention provides compositions and methods for treatment of diseases with unwanted angiogenesis, often an abnormal or excessive proliferation and growth of blood vessels. Since angiogenesis also can be a normal biological process, inhibition of unwanted angiogenesis is preferably accomplished with selectivity for a pathological tissue, which preferably requires selective delivery of therapeutic molecules to the pathological tissue using targeted nanoparticles. The present invention provides compositions and methods to control angiogenesis through selective inhibition of the Ang-Tie biochemical pathway by nucleic acid molecules that induce RNA interference (RNAi), including inhibition of Ang-Tie pathway gene expression and inhibition localized at pathological angiogenic tissues. The present invention also provides compositions of and methods for using a tissue-targeted nanoparticle composition comprising polymer conjugates and further comprising nucleic acid molecules that induce RNAi.

The invention is described here in detail, but one skilled in the art will appreciate the full extent of the invention.

### Nucleic Acid Molecules for Ang/Tie2Pathway Gene Inhibition

The present invention provides nucleic acid molecules with a variety of physicochemical structures for targeting and silencing genes in the Ang/Tie2 pathway by RNAi. In one embodiment, the present invention provides nucleic acid molecules that result in a reduction in Ang-1, Ang-2, or Tie2 mRNA or protein levels of at least 50%, 60%, 70%, 80%, 85%, 90%, 95, 96, 97, 98, 99 or 100%. This reduction may result up to 24 hours, up to 36 hours, up to 48 hours, up to 60 hours, or up to 72 hours post administration of the nucleic acid molecules. The nucleic acid molecules that result in this reduction may be administered at 10 nM siRNA, 5 nM siRNA, 2 nM, 1 nM, 0.5 nM, or 0.2 nM quantities. In one embodiment, the nucleic acid molecules may have an IC50 for reducing Ang-2 protein levels of 0.75 nM or less, 0.5 nM or less, or 0.4 nM or less.

The nucleic acid molecules of the invention may be dsRNA or ssRNA. In one embodiment of the invention, the nucleic acid molecules are siRNA. The nucleic acid molecules may comprise 15-50, 15-30, 19, 20, 21, 22, 23, 24 or 25 base pairs. The nucleic acid molecules may comprise 5′- or 3′-single-stranded overhangs. In a certain embodiment, the nucleic acid molecules are blunt-ended. In a preferred embodiment, the nucleic acid molecule is a double-stranded siRNA of 25 basepairs with blunt ends. Exemplary siRNA sequences of the invention targeting Ang/Tie2 pathway genes are shown in Tables 2-10. (For all sequences listed in Tables 2-10, the position is labeled such that the “A” of the ATG codon is considered to be position 1.) siRNAs with 25 basepair double-stranded RNA with blunt ends were previously found to be some of the most potent inhibitors with the greatest duration of inhibition (WO 06/110813). Additionally, incorporation of non-naturally occurring chemical analogues may be useful in some embodiments of the invention. Such analogues include, but are not limited to, 2′-O-Methyl ribose analogues of RNA, DNA, LNA and RNA chimeric oligonucleotides, and other chemical analogues of nucleic acid oligonucleotides. In some embodiments, the siRNA targets both a human mRNA as well as the homologous or analogous mRNA in other non-human mammalian species such as primates, mice or rats.

TABLE 2
siRNA candidates for human TEK (Tie-2) gene.

| | siRNA Sequence (sense | | SEQ ID |
|---|---|---|---|
| Start | strand/anti-sense strand) | GC % | NO: |
| 67 | 5′-GCCAUGGACUUGAUCUUGAUCAAUU-3′ | 40.0 | 1 |
| | 3′-CGGUACCUGAACUAGAACUAGUUAA-5′ | | 2 |
| 93 | 5′-CCUACCUCUUGUAUCUGAUGCUGAA-3′ | 44.0 | 3 |
| | 3′-GGAUGGAGAACAUAGACUACGACUU-5′ | | 4 |
| 498 | 5′-CCGGCAUGAAGUACCUGAUAUUCUA-3′ | 44.0 | 5 |
| | 3′-GGCCGUACUUCAUGGACUAUAAGAU-5′ | | 6 |
| 744 | 5′-AAGGACGUGUGAGAAGGCUUGUGAA-3′ | 48.0 | 7 |
| | 3′-UUCCUGCACACUCUUCCGAACACUU-5′ | | 8 |
| 1372 | 5′-CAUAACUUUGCUGUCAUCAACAUCA-3′ | 36.0 | 9 |
| | 3′-GUAUUGAAACGACAGUAGUUGUAGU-5′ | | 10 |
| 1784 | 5′-GCAACUUGACUUCGGUGCUACUUAA-3′ | 44.0 | 11 |
| | 3′-CGUUGAACUGAAGCCACGAUGAAUU-5′ | | 12 |
| 1975 | 5′-UGGACAAUAUUGGAUGGCUAUUCUA-3′ | 36.0 | 13 |
| | 3′-ACCUGUUAUAACCUACCGAUAAGAU-5′ | | 14 |
| 2609 | 5′-CAGGAGAACUGGAAGUUCUUUGUAA-3′ | 40.0 | 15 |
| | 3′-GUCCUCUUGACCUUCAAGAAACAUU-5′ | | 16 |
| 2655 | 5′-CAUCAAUCUCUUAGGAGCAUGUGAA-3′ | 40.0 | 17 |
| | 3′-GUAGUUAGAGAAUCCUCGUACACUU-5′ | | 18 |
| 3231 | 5′-GAAGCCUUAUGAGAGGCCAUCAUUU-3′ | 44.0 | 19 |
| | 3′-CUUCGGAAUACUCUCCGGUAGUAAA-5′ | | 20 |
| 204 | 5′-CCAGGAUCCGCUGGAAGUUACUCAA-3′ | 52.0 | 21 |
| | 3′-GGUCCUAGGCGACCUUCAAUGAGUU-5′ | | 22 |
| 319 | 5′-CGAGGAGAGGCAAUCAGGAUACGAA-3′ | 52.0 | 23 |
| | 3′-GCUCCUCUCCGUUAGUCCUAUGCUU-5′ | | 24 |
| 351 | 5′-GAUGCGUCAACAAGCUUCCUUCCUA-3′ | 48.0 | 25 |
| | 3′-CUACGCAGUUGUUCGAAGGAAGGAU-5′ | | 26 |
| 363 | 5′-AGCUUCCUUCCUACCAGCUACUUUA-3′ | 44.0 | 27 |
| | 3′-UCGAAGGAAGGAUGGUCGAUGAAAU-5′ | | 28 |
| 400 | 5′-GACAAGGGAGAUAACGUGAACAUAU-3′ | 40.0 | 29 |
| | 3′-CUGUUCCCUCUAUUGCACUUGUAUA-5′ | | 30 |
| 612 | 5′-CAGGCUGAUAGUCCGGAGAUGUGAA-3′ | 52.0 | 31 |
| | 3′-GUCCGACUAUCAGGCCUCUACACUU-5′ | | 32 |
| 660 | 5′-CAACCAUCUCUGUACUGCUGUAUG-3′ | 44.0 | 33 |
| | 3′-GUUGGUAGAGACAUGACGACAUAC-5′ | | 34 |
| 664 | 5′-CAUCUCUGUACUGCUUGUAUGAACA-3′ | 40.0 | 35 |
| | 3′-GUAGAGACAUGACGAACAUACUUGU-5′ | | 36 |
| 771 | 5′-GCACACGUUUGGCAGAACUUGUAAA-3′ | 44.0 | 37 |
| | 3′-CGUGUGCAAACCGUCUUGAACAUUU-5′ | | 38 |
| 805 | 5′-AGUGGACAAGAGGGAUGCAAGUCUU-3′ | 48.0 | 39 |
| | 3′-UCACCUGUUCUCCCUACGUUCAGAA-5′ | | 40 |
| 812 | 5′-AAGAGGGAUGCAAGUCUUAUGUGUU-3′ | 40.0 | 41 |
| | 3′-UUCUCCCUACGUUCAGAAUACACAA-5′ | | 42 |
| 893 | 5′-GCAAUGAAGCAUGCCACCCUGGUUU-3′ | 52.0 | 43 |
| | 3′-CGUUACUUCGUACGGUGGGACCAAA-5′ | | 44 |
| 1049 | 5′-CAAAGAUAGUGGAUUUGCCAGAUCA-3′ | 40.0 | 45 |
| | 3′-GUUUCUAUCACCUAAACGGUCUAGU-5′ | | 46 |
| 1053 | 5′-GAUAGUGGAUUUGCCAGAUCAUAUA-3′ | 36.0 | 47 |
| | 3′-CUAUCACCUAAACGGUCUAGUAUAU-5′ | | 48 |
| 1369 | 5′-GGACAUAACUUUGCUGUCAUCAACA-3′ | 40.0 | 49 |
| | 3′-CCUGUAUUGAAACGACAGUAGUUGU-5′ | | 50 |
| 1455 | 5′-CGUUAAUCACUAUGAGGCUUGGCAA-3′ | 44.0 | 51 |
| | 3′-GCAAUUAGUGAUACUCCGAACCGUU-5′ | | 52 |
| 1463 | 5′-ACUAUGAGGCUUGGCAACAUAUUCA-3′ | 40.0 | 53 |
| | 3′-UGAUACUCCGAACCGUUGUAUAAGU-5′ | | 54 |
| 1636 | 5′-CCAAGAGGUCUAAAUCUCCUGCCUA-3′ | 48.0 | 55 |
| | 3′-GGUUCUCCAGAUUUAGAGGACGGAU-5′ | | 56 |
| 1637 | 5′-CAAGAGGUCUAAAUCUCCUGCCUAA-3′ | 44.0 | 57 |
| | 3′-GUUCUCCAGAUUUAGAGGACGGAUU-5′ | | 58 |
| 1763 | 5′-AGCAGAAUAUUAAAGUUCCAGGCAA-3′ | 36.0 | 59 |
| | 3′-UCGUCUUAUAAUUUCAAGGUCCGUU-5′ | | 60 |
| 1781 | 5′-CAGGCAACUUGACUUCGGUGCUACU-3′ | 52.0 | 61 |
| | 3′-GUCCGUUGAACUGAAGCCACGAUGA-5′ | | 62 |
| 1879 | 5′-GAAGAUCUCACUGCUUGGACCCUUA-3′ | 48.0 | 63 |
| | 3′-CUUCUAGAGUGACGAACCUGGGAAU-5′ | | 64 |
| 1898 | 5′-CCCUUAGUGACAUUCUUCCUCCUCA-3′ | 48.0 | 65 |
| | 3′-GGGAAUCACUGUAAGAAGGAGGAGU-5′ | | 66 |
| 1899 | 5′-CCUUAGUGACAUUCUUCCUCCUCAA-3′ | 44.0 | 67 |
| | 3′-GGAAUCACUGUAAGAAGGAGGAGUU-5′ | | 68 |
| 2610 | 5′-AGGAGAACUGGAAGUUCUUUGUAAA-3′ | 36.0 | 69 |
| | 3′-UCCUCUUGACCUUCAAGAAACAUUU-5′ | | 70 |
| 2684 | 5′-GAGGCUACUUGUACCUGGCCAUUGA-3′ | 52.0 | 71 |
| | 3′-CUCCGAUGAACAUGGACCGGUAACU-5′ | | 72 |
| 2723 | 5′-GAAACCUUCUGGACUUCCUUCGCAA-3′ | 48.0 | 73 |
| | 3′-CUUUGGAAGACCUGAAGGAAGCGUU-5′ | | 74 |
| 3020 | 5′-UCGAGUCACUGAAUUACAGUGUGUA-3′ | 40.0 | 75 |
| | 3′-AGCUCAGUGACUUAAUGUCACACAU-5′ | | 76 |
| 3119 | 5′-GCGGGAUGACUUGUGCAGAACUCUA-3′ | 52.0 | 77 |
| | 3′-CGCCCUACUGAACACGUCUUGAGAU-5′ | | 78 |
| 3179 | 5′-CCCUGAACUGUGAUGAUGAGGUGUA-3′ | 48.0 | 79 |
| | 3′-GGGACUUGACACUACUACUCCACAU-5′ | | 80 |
| 3289 | 5′-GAGGAGCGAAAGACCUACGUGAAUA-3′ | 48.0 | 81 |
| | 3′-CUCCUCGCUUUCUGGAUGCACUUAU-5′ | | 82 |
| 72 | 5′-GGACUUGAUCUUGAUCAAUUCCCUA-3′ | 40.0 | 83 |
| | 3′-CCUGAACUAGAACUAGUUAAGGGAU-5′ | | 84 |
| 77 | 5′-UGAUCUUGAUCAAUUCCCUACCUCU-3′ | 40.0 | 85 |
| | 3′-ACUAGAACUAGUUAAGGGAUGGAGA-5′ | | 86 |
| 87 | 5′-CAAUUCCCUACCUCUUGUAUCUGAU-3′ | 40.0 | 87 |
| | 3′-GUUAAGGGAUGGAGAACAUAGACUA-5′ | | 88 |
| 207 | 5′-GGAUCCGCUGGAAGUUACUCAAGAU-3′ | 48.0 | 89 |
| | 3′-CCUAGGCGACCUUCAAUGAGUUCUA-5′ | | 90 |
| 326 | 5′-AGGCAAUCAGGAUACGAACCAUGAA-3′ | 44.0 | 91 |
| | 3′-UCCGUUAGUCCUAUGCUUGGUACUU-5′ | | 92 |
| 406 | 5′-GGAGAUAACGUGAACAUAUCUUUCA-3′ | 36.0 | 93 |
| | 3′-CCUCUAUUGCACUUGUAUAGAAAGU-5′ | | 94 |
| 571 | 5′-GCCAGGUAUAUAGGAGGAAACCUCU-3′ | 48.0 | 95 |
| | 3′-CGGUCCAUAUAUCCUCCUUUGGAGA-5′ | | 96 |
| 572 | 5′-CCAGGUAUAUAGGAGGAAACCUCUU-3′ | 44.0 | 97 |
| | 3′-GGUCCAUAUAUCCUCCUUUGGAGAA-5′ | | 98 |
| 693 | 5′-UGUCUGCCAUGAAGAUACUGGAGAA-3′ | 44.0 | 99 |
| | 3′-ACAGACGGUACUUCUAUGACCUCUU-5′ | | 100 |
| 774 | 5′-CACGUUUGGCAGAACUUGUAAAGAA-3′ | 40.0 | 101 |
| | 3′-GUGCAAACCGUCUUGAACAUUUCUU-5′ | | 102 |
| 807 | 5′-UGGACAAGAGGGAUGCAAGUCUUAU-3′ | 44.0 | 103 |
| | 3′-ACCUGUUCUCCCUACGUUCAGAAUA-5′ | | 104 |
| 961 | 5′-GAGAUGUGUGAUCGCUUCCAAGGAU-3′ | 48.0 | 105 |
| | 3′-CUCUACACACUAGCGAAGGUUCCUA-5′ | | 106 |
| 970 | 5′-GAUCGCUUCCAAGGAUGUCUCUGCU-3′ | 52.0 | 107 |
| | 3′-CUAGCGAAGGUUCCUACAGAGACGA-5′ | | 108 |
| 1352 | 5′-CAAACGUGAUUGACACUGGACAUAA-3′ | 40.0 | 109 |
| | 3′-GUUUGCACUAACUGUGACCUGUAUU-5′ | | 110 |
| 1364 | 5′-ACACUGGACAUAACUUUGCUGUCAU-3′ | 40.0 | 111 |
| | 3′-UGUGACCUGUAUUGAAACGACAGUA-5′ | | 112 |
| 1385 | 5′-UCAUCAACAUCAGCUCUGAGCCUUA-3′ | 44.0 | 113 |
| | 3′-AGUAGUUGUAGUCGAGACUCGGAAU-5′ | | 114 |
| 1388 | 5′-UCAACAUCAGCUCUGAGCCUUACUU-3′ | 44.0 | 115 |
| | 3′-AGUUGUAGUCGAGACUCGGAAUGAA-5′ | | 116 |
| 1389 | 5′-CAACAUCAGCUCUGAGCCUUACUUU-3′ | 44.0 | 117 |
| | 3′-GUUGUAGUCGAGACUCGGAAUGAAA-5′ | | 118 |
| 1436 | 5′-AGAAGCUUCUAUACAAACCCGUUAA-3′ | 36.0 | 119 |
| | 3′-UCUUCGAAGAUAUGUUUGGGCAAUU-5′ | | 120 |
| 1437 | 5′-GAAGCUUCUAUACAAACCCGUUAAU-3′ | 36.0 | 121 |
| | 3′-CUUCGAAGAUAUGUUUGGGCAAUUA-5′ | | 122 |
| 1454 | 5′-CCGUUAAUCACUAUGAGGCUUGGCA-3′ | 48.0 | 123 |
| | 3′-GGCAAUUAGUGAUACUCCGAACCGU-5′ | | 124 |
| 1668 | 5′-GACCACUCUAAAUUUGACCUGGCAA-3′ | 44.0 | 125 |
| | 3′-CUGGUGAGAUUUAAACUGGACCGUU-5′ | | 126 |
| 1791 | 5′-GACUUCGGUGCUACUUAACAACUUA-3′ | 40.0 | 127 |
| | 3′-CUGAAGCCACGAUGAAUUGUUGAAU-5′ | | 128 |
| 1951 | 5′-ACACACUCCUCGGCUGUGAUUUCUU-3′ | 48.0 | 129 |
| | 3′-UGUGUGAGGAGCCGACACUAAAGAA-5′ | | 130 |
| 2050 | 5′-CACGUUGAUGUGAAGAUAAAGAAUG-3′ | 36.0 | 131 |
| | 3′-GUGCAACUACACUUCUAUUUCUUAC-5′ | | 132 |
| 2061 | 5′-GAAGAUAAAGAAUGCCACCAUCAUU-3′ | 36.0 | 133 |
| | 3′-CUUCUAUUUCUUACGGUGGUAGUAA-5′ | | 134 |
| 2141 | 5′-CAGAGAACAACAUAGGGUCAAGCAA-3′ | 44.0 | 135 |
| | 3′-GUCUCUUGUUGUAUCCCAGUUCGUU-5′ | | 136 |
| 2232 | 5′-GAAGAUGCUGCUUAUAGCCAUCCUU-3′ | 44.0 | 137 |
| | 3′-CUUCUACGACGAAUAUCGGUAGGAA-5′ | | 138 |
| 2246 | 5′-UAGCCAUCCUUGGCUCUGCUGGAAU-3′ | 52.0 | 139 |
| | 3′-AUCGGUAGGAACCGAGACGACCUUA-5′ | | 140 |
| 2387 | 5′-UCAACUCAGGGACUCUGGCCCUAAA-3′ | 52.0 | 141 |
| | 3′-AGUUGAGUCCCUGAGACCGGGAUUU-5′ | | 142 |
| 2398 | 5′-ACUCUGGCCCUAAACAGGAAGGUCA-3′ | 52.0 | 143 |
| | 3′-UGAGACCGGGAUUUGUCCUUCCAGU-5′ | | 144 |
| 2603 | 5′-ACUUUGCAGGAGAACUGGAAGUUCU-3′ | 44.0 | 145 |
| | 3′-UGAAACGUCCUCUUGACCUUCAAGA-5′ | | 146 |
| 2608 | 5′-GCAGGAGAACUGGAAGUUCUUUGUA-3′ | 44.0 | 147 |
| | 3′-CGUCCUCUUGACCUUCAAGAAACAU-5′ | | 148 |
| 2618 | 5′-UGGAAGUUCUUUGUAAACUUGGACA-3′ | 36.0 | 149 |
| | 3′-ACCUUCAAGAAACAUUUGAACCUGU-5′ | | 150 |
| 2722 | 5′-GGAAACCUUCUGGACUUCCUUCGCA-3′ | 52.0 | 151 |
| | 3′-CCUUUGGAAGACCUGAAGGAAGCGU-5′ | | 152 |
| 2767 | 5′-GACCCAGCAUUUGCCAUDGCCAAUA-3′ | 48.0 | 153 |
| | 3′-CUGGGUCGUAAACGGUAACGGUUAU-5′ | | 154 |
| 2958 | 5′-CCGAGGUCAAGAGGUGUACGUGAAA-3′ | 52.0 | 155 |
| | 3′-GGCUCCAGUUCUCCACAUGCACUUU-5′ | | 156 |
| 3072 | 5′-UGGUGUGUUACUAUGGGAGAUUGUU-3′ | 40.0 | 157 |
| | 3′-ACCACACAAUGAUACCCUCUAACAA-5′ | | 158 |
| 3073 | 5′-GGUGUGUUACUAUGGGAGAUUGUUA-3′ | 40.0 | 159 |
| | 3′-CCACACAAUGAUACCCUCUAACAAU-5′ | | 160 |
| 3298 | 5′-AAGACCUACGUGAAUACCACGCUUU-3′ | 44.0 | 161 |
| | 3′-UUCUGGAUGCACUUAUGGUGCGAAA-5′ | | 162 |
| 3300 | 5′-GACCUACGUGAAUACCACGCUUUAU-3′ | 44.0 | 163 |
| | 3′-CUGGAUGCACUUAUGGUGCGAAAUA-5′ | | 164 |
| 3314 | 5′-CCACGCUUUAUGAGAAGUUUACUUA-3′ | 36.0 | 165 |
| | 3′-GGUGCGAAAUACUCUUCAAAUGAAU-5′ | | 166 |

TABLE 3
siRNA candidates for mouse Tie2 gene.

| | | | SEQ |
|---|---|---|---|
| | siRNA Sequence (sense | | ID |
| Start | strand/anti-sense strand) | GC % | NO: |
| 612 | 5′-CAGGCUGAUUGUUCGGAGAUGUGAA-3′ | 48.0 | 171 |
| | 3′-GUCCGACUAACAAGCCUCUACACUU)-5′ | | 172 |
| 664 | 5′-CGUCCUUGUACUACUUGCAAGAACA-3′ | 44.0 | 173 |
| | 3′-GCAGGAACAUGAUGAACGUUCUUGU-5′ | | 174 |
| 756 | 5′-GAAAGCUUGUGAGCCGCACACAUUU-3′ | 48.0 | 175 |
| | 3′-CUUUCGAACACUCGGCGUGUGUAAA-5′ | | 176 |
| 812 | 5′-CAGAAGGAUGCAAGUCUUAUGUGUU-3′ | 40.0 | 173 |
| | 3′-GUCUUCCUACGUUCAGAAUACACAA-5′ | | 174 |
| 1032 | 5′-CAGGCCAAGGAUGACUCCACAGAUA-3′ | 52.0 | 175 |
| | 3′-GUCCGGUUCCUACUGAGGUGUCUAU-5′ | | 176 |
| 1049 | 5′-CACAGAUAGAGGAUUUGCCAGAUCA-3′ | 44.0 | 177 |
| | 3′-GUGUCUAUCUCCUAAACGGUCUAGU-5′ | | 178 |
| 1119 | 5′-UGGGUGGCCACUACCUACUAGUGAA-3′ | 52.0 | 179 |
| | 3′-ACCCACCGGUGAUGGAUGAUCACUU-5′ | | 180 |
| 1631 | 5′-CAAGAGGUCUCAGUCUCCUGCCAAA-3′ | 52.0 | 181 |
| | 3′-GUUCUCCAGAGUCAGAGGACGGUUU-5′ | | 182 |
| 1734 | 5′-GCGAUCCCUGCAAACAACAAGUGAU-3′ | 48.0 | 183 |
| | 3′-CGCUAGGGACGUUUGUUGUUCACUA-5′ | | 184 |
| 1760 | 5′-AGCAGAACAUCAAAGUGCCUGGGAA-3′ | 48.0 | 185 |
| | 3′-UCGUCUUGUAGUUUCACGGACCCUU-5′ | | 186 |
| 62 | 5′-AAGGUGCCAUGGACCUGAUCUUGAU-3′ | 48.0 | 187 |
| | 3′-UUCCACGGUACCUGGACUAGAACUA-5′ | | 188 |
| 67 | 5′-GCCAUGGACCUGAUCUUGAUCAAUU-3′ | 44.0 | 189 |
| | 3′-CGGUACCUGGACUAGAACUAGUUAA-5′ | | 190 |
| 93 | 5′-CCUACCUCUUGUGUCUGAUGCCGAA-3′ | 52.0 | 191 |
| | 3′-GGAUGGAGAACACAGACUACGGCUU-5′ | | 192 |
| 162 | 5′-CAUCACCAUAGGAAGGGACUUUGAA-3′ | 44.0 | 193 |
| | 3′-GUAGUGGUAUCCUUCCCUGAAACUU-5′ | | 194 |
| 204 | 5′-CCAAGAUCCACUGGAGGUUACUCAA-3′ | 48.0 | 195 |
| | 3′-GGUUCUAGGUGACCUCCAAUGAGUU-5′ | | 196 |
| 276 | 5′-GGCCAGUAAGAUUAAUGGUGCUUAU-3′ | 40.0 | 197 |
| | 3′-CCGGUCAUUCUAAUUACCACGAAUA-5′ | | 198 |
| 351 | 5′-GAUGCGUCAACAAGCGUCCUUCCUA-3′ | 52.0 | 199 |
| | 3′-CUACGCAGUUGUUCGCAGGAAGGAU-5′ | | 200 |
| 363 | 5′-AGCGUCCUUCCUACCUGCUACUUUA-3′ | 48.0 | 201 |
| | 3′-UCGCAGGAAGGAUGGACGAUGAAAU-5′ | | 202 |
| 572 | 5′-CCAGGUACAUAGGAGGAAACCUGUU-3′ | 48.0 | 203 |
| | 3′-GGUCCAUGUAUCCUCCUUUGGACAA-5′ | | 204 |
| 654 | 5′-CGACUGUAGCCGUCCUUGUACUACU-3′ | 52.0 | 205 |
| | 3′-GCUGACAUCGGCAGGAACAUGAUGA-5′ | | 206 |
| 744 | 5′-GAGAACAUGUGAGAAAGCUUGUGAG-3′ | 44.0 | 207 |
| | 3′-CUCUUGUACACUCUUUCGAACACUC-5′ | | 208 |
| 756 | 5′-GAAAGCUUGUGAGCCGCACACAUUU-3′ | 48.0 | 209 |
| | 3′-CUUUCGAACACUCGGCGUGUGUAAA-5′ | | 210 |
| 770 | 5′-CGCACACAUUUGGCAGGACCUGUAA-3′ | 52.0 | 211 |
| | 3′-GCGUGUGUAAACCGUCCUGGACAUU-5′ | | 212 |
| 771 | 5′-GCACACAUUUGGCAGGACCUGUAAA-3′ | 48.0 | 213 |
| | 3′-CGUGUGUAAACCGUCCUGGACAUUU-5′ | | 214 |
| 805 | 5′-AGUGGACCAGAAGGAUGCAAGUCUU-3′ | 48.0 | 215 |
| | 3′-UCACCUGGUCUUCCUACGUUCAGAA-5′ | | 216 |
| 928 | 5′-GACUGUAAGCUCAGGUGCCACUGUA-3′ | 52.0 | 217 |
| | 3′-CUGACAUUCGAGUCCACGGUGACAU-5′ | | 218 |
| 1233 | 5′-CAACCGAGUCUUACCUCCUGACUCA-3′ | 52.0 | 219 |
| | 3′-GUUGGAUCAGAAUGGAGGACUGAGU-5′ | | 220 |
| 1453 | 5′-CCUGUCAAUCAGGCCUGGAAAUACA-3′ | 48.0 | 221 |
| | 3′-GGACAGUUAGUCCGGACCUUUAUGU-5′ | | 222 |
| 1458 | 5′-CAAUCAGGCCUGGAAAUACAUUGAA-3′ | 40.0 | 223 |
| | 3′-GUUAGUCCGGACCUUUAUGUAACUU-5′ | | 224 |
| 1956 | 5′-CACAGCUAUGGUUUCUUGGACAAUA-3′ | 40.0 | 225 |
| | 3′-GUGUCGAUACCAAAGAACCUGUUAU-5′ | | 226 |
| 2041 | 5′-GACCAGCACAUUGAUGUGAAGAUCA-3′ | 44.0 | 227 |
| | 3′-CUGGUCGUGUAACUACACUUCUAGU-5′ | | 228 |
| 2047 | 5′-CACAUUGAUGUGAAGAUCAAGAAUG-3′ | 36.0 | 229 |
| | 3′-GUGUAACUACACUUCUAGUUCUUAC-5′ | | 230 |
| 2100 | 5′-CCUAGAGCCAGAGACUACAUACCAU-3′ | 48.0 | 231 |
| | 3′-GGAUCUCGGUCUCUGAUGUAUGGUA-5′ | | 232 |
| 2418 | 5′-AAACAAUCCGGAUCCCACAAUUUAU-3′ | 36.0 | 233 |
| | 3′-UUUGUUAGGCCUAGGGUGUUAAAUA-5′ | | 234 |
| 2456 | 5′-GGAAUGACAUCAAGUUUCAAGACGU-3′ | 40.0 | 235 |
| | 3′-CCUUACUGUAGUUCAAAGUUCUGCA-5′ | | 236 |
| 2549 | 5′-CCGCCAUCAAGAGGAUGAAAGAGUA-3′ | 48.0 | 237 |
| | 3′-GGCGGUAGUUCUCCUACUUUCUCAU-5′ | | 238 |
| 2559 | 5′-GAGGAUGAAAGAGUAUGCCUCCAAA-3′ | 44.0 | 239 |
| | 3′-CUAAUACUUUCUCAUACGGAGGUUU-5′ | | 240 |
| 2602 | 5′-GCAGGAGAACUGGAGGUUCUUUGUA-3′ | 48.0 | 241 |
| | 3′-CGUCCUCUUGACCUCCAAGAAACAU-5′ | | 242 |
| 2603 | 5′-CAGGAGAACUGGAGGUUCUUUGUAA-3′ | 44.0 | 243 |
| | 3′-GUCCUCUUGACCUCCAAGAAACAUU-5′ | | 244 |
| 2604 | 5′-AGGAGAACUGGAGGUUCUUUGUAAA-3′ | 40.0 | 245 |
| | 3′-UCCUCUUGACCUCCAAGAAACAUUU-5′ | | 246 |
| 2649 | 5′-CAUCAAUCUCUUGGGAGCAUGUGAA-3′ | 44.0 | 247 |
| | 3′-GUAGUUAGAGAACCCUCGUACACUU-5′ | | 248 |
| 2674 | 5′-CACCGAGGCUAUUUGUACCUAGCUA-3′ | 48.0 | 249 |
| | 3′-GUGGCUCCGAUAAACAUGGAUCGAU-5′ | | 250 |
| 2676 | 5′-CCGAGGCUAUUUGUACCUAGCUAUU-3′ | 44.0 | 251 |
| | 3′-GGCUCCGAUAAACAUGGAUCGAUAA-5′ | | 252 |
| 2678 | 5′-GAGGCUAUUUGUACCUAGCUAUUGA-3′ | 40.0 | 253 |
| | 3′-CUCCGAUAAACAUGGAUCGAUAACU-5′ | | 254 |
| 2945 | 5′-GAUUGUCACGAGGUCAAGAAGUGUA-3′ | 44.0 | 255 |
| | 3′-CUAACAGUGCUCCAGUUCUUCACAU-5′ | | 256 |
| 2951 | 5′-CACGAGGUCAAGAAGUGUAUGUGAA-3′ | 44.0 | 257 |
| | 3′-GUGCUCCAGUUCUUCACAUACACUU-5′ | | 258 |
| 2995 | 5′-CCAGUGCGUUGGAUGGCAAUCGAAU-3′ | 52.0 | 259 |
| | 3′-GGUCACGCAACCUACCGUUAGCUUA-5′ | | 260 |
| 3309 | 5′-CACACUGUAUGAGAAGUUUACCUAU-3′ | 36.0 | 261 |
| | 3′-GUGUGACAUACUCUUCAAAUGGAUA-5′ | | 262 |

TABLE 4
siRNA candidates for human/mouse TEK (Tie-2).

| | siRNA Sequence | | SEQ |
|---|---|---|---|
| | (sense strand/ | | ID |
| Start | anti-sense strand) | GC % | NO: |
| 77 | 5′-UGAUCUUGAUCAAUUCCCUACCUCU-3′ | 40.0 | 263 |
| | 3′-ACUAGAACUAGUUAAGGGAUGGAGA-5′ | | 264 |
| 161 | 5′-CCAUCACCAUAGGAAGGGACUUUGA-3′ | 48.0 | 265 |
| | 3′-GGUAGUGGUAUCCUUCCCUGAAACU-5′ | | 266 |
| 162 | 5′-CAUCACCAUAGGAAGGGACUUUGAA-3′ | 44.0 | 267 |
| | 3′-GUAGUGGUAUCCUUCCCUGAAACUU-5′ | | 268 |
| 3179 | 5′-CCCUGAACUGUGAUGAUGAGGUGUA-3′ | 48.0 | 269 |
| | 3′-GGGACUUGACACUACUACUCCACAU-5′ | | 270 |

TABLE 5
siRNA candidates for human ANGPT1.

| | siRNA Sequence | | SEQ |
|---|---|---|---|
| | (sense strand/ | | ID |
| Start | anti-sense strand) | GC % | NO: |
| 842 | 5′-CAUUUAGAGACUGUGCAGAUGUAUA-3′ | 36.0 | 271 |
| | 3′-GUAAAUCUCUGACACGUCUACAUAU-5′ | | 272 |
| 978 | 5′-ACAACAUCGUGAAGAUGGAAGUCUA-3′ | 40.0 | 273 |
| | 3′-UGUUGUAGCACUUCUACCUUCAGAU-5′ | | 274 |
| 1003 | 5′-GAUUUCCAAAGAGGCUGGAAGGAAU-3′ | 44.0 | 275 |
| | 3′-CUAAAGGUUUCUCCGACCUUCCUUA-5′ | | 276 |
| 1116 | 5′-AAGAAUUGAGUUAAUGGACUGGGAA-3′ | 36.0 | 277 |
| | 3′-UUCUUAACUCAAUUACCUGACCCUU-5′ | | 278 |
| 1245 | 5′-CAGCCUGAUCUUACACGGUGCUGAU-3′ | 52.0 | 279 |
| | 3′-GUCGGACUAGAAUGUGCCACGACUA-5′ | | 280 |
| 1357 | 5′-CCCUCCAAUCUAAAUGGAAUGUUCU-3′ | 40.0 | 281 |
| | 3′-GGGAGGUUAGAUUUACCUUACAAGA-5′ | | 282 |
| 1358 | 5′-CCUCCAAUCUAAAUGGAAUGUUCUA-3 | 36.0 | 283 |
| | 3′-GGAGGUUAGAUUUACCUUACAAGAU-5′ | | 284 |
| 1443 | 5′-CAGUUACUCCUUACGUUCCACAACU-3′ | 44.0 | 285 |
| | 3′-GUCAAUGAGGAAUGCAAGGUGUUGA-5′ | | 286 |
| 1460 | 5′-CCACAACUAUGAUGAUUCGACCUUU-3′ | 40.0 | 287 |
| | 3′-GGUGUUGAUACUACUAAGCUGGAAA-5′ | | 288 |
| 1461 | 5′-CACAACUAUGAUGAUUCGACCUUUA-3′ | 36.0 | 289 |
| | 3′-GUGUUGAUACUACUAAGCUGGAAAU-5′ | | 290 |
| 89 | 5′-GGAGAAGAUAUAACCGGAUUCAACA-3′ | 40.0 | 291 |
| | 3′-CCUCUUCUAUAUUGGCCUAAGUUGU-5′ | | 292 |
| 109 | 5′-CAACAUGGGCAAUGUGCCUACACUU-3′ | 48.0 | 293 |
| | 3′-GUUGUACCCGUUACACGGAUGUGAA-5′ | | 294 |
| 112 | 5′-CAUGGGCAAUGUGCCUACACUUUCA-3′ | 48.0 | 295 |
| | 3′-GUACCCGUUACACGGAUGUGAAAGU-5′ | | 296 |
| 125 | 5′-CCUACACUUUCAUUCUUCCAGAACA-3′ | 40.0 | 297 |
| | 3′-GGAUGUGAAAGUAAGAAGGUCUUGU-5′ | | 298 |
| 346 | 5′-CAGCAGAAUGCAGUUCAGAACCACA-3′ | 48.0 | 299 |
| | 3′-GUCGUCUUACGUCAAGUCUUGGUGU-5′ | | 300 |
| 654 | 5′-CCUUCAAGGCUUGGUUACUCGUCAA-3′ | 48.0 | 301 |
| | 3′-GGAAGUUCCGAACCAAUGAGCAGUU-5′ | | 302 |
| 1159 | 5′-CAGUAUGACAGAUUCCACAUAGGAA-3′ | 40.0 | 303 |
| | 3′-GUCAUACUGUCUAAGGUGUAUCCUU-5′ | | 304 |
| 1328 | 5′-CAGGAGGAUGGUGGUUUGAUGCUUG-3′ | 52.0 | 305 |
| | 3′-GUCCUCCUACCACCAAACUACGAAC-5′ | | 306 |
| 95 | 5′-GAUAUAACCGGAUUCAACAUGGGCA-3′ | 44.0 | 307 |
| | 3′-CUAUAUUGGCCUAAGUUGUACCCGU-5′ | | 308 |
| 108 | 5′-UCAACAUGGGCAAUGUGCCUACACU-3′ | 48.0 | 309 |
| | 3′-AGUUGUACCCGUUACACGGAUGUGA-5′ | | 310 |
| 437 | 5′-CAGAUGUUGAGACCCAGGUACUAAA-3′ | 44.0 | 311 |
| | 3′-GUCUACAACUCUGGGUCCAUGAUUU-5′ | | 312 |
| 1168 | 5′-GACAGAUUCCACAUAGGAAAUGAAA-3′ | 36.0 | 313 |
| | 3′-CUGUCUAAGGUGUAUCCUUUACUUU-5′ | | 314 |
| 1412 | 5′-UGAAUGGGAUAAAGUGGCACUACUU-3′ | 40.0 | 315 |
| | 3′-ACUUACCCUAUUUCACCGUGAUGAA-5′ | | 316 |
| 1427 | 5′-GGCACUACUUCAAAGGGCCCAGUUA-3′ | 52.0 | 317 |
| | 3′-CCGUGAUGAAGUUUCCCGGGUCAAU-5′ | | 318 |
| 163 | 5′-CGUGAGAGUACGACAGACCAGUACA-3′ | 52.0 | 319 |
| | 3′-GCACUCUCAUGCUGUCUGGUCAUGU-5′ | | 320 |
| 166 | 5′-GAGAGUACGACAGACCAGUACAACA-3′ | 48.0 | 321 |
| | 3′-CUCUCAUGCUGUCUGGUCAUGUUGU-5′ | | 322 |
| 176 | 5′-CAGACCAGUACAACACAAACGCUCU-3′ | 48.0 | 323 |
| | 3′-GUCUGGUCAUGUUGUGUUUGCGAGA-5′ | | 324 |
| 213 | 5′-UCCACACGUGGAACCGGAUUUCUCU-3′ | 52.0 | 325 |
| | 3′-AGGUGUGCACCUUGGCCUAAAGAGA-5′ | | 326 |
| 214 | 5′-CCACACGUGGAACCGGAUUUCUCUU-3′ | 52.0 | 327 |
| | 3′-GGUGUGCACCUUGGCCUAAAGAGAA-5′ | | 328 |
| 250 | 5′-CAACAUCUGGAACAUGUGAUGGAAA-3′ | 40.0 | 329 |
| | 3′-GUUGUAGACCUUGUACACUACCUUU-5′ | | 330 |
| 336 | 5′-GGCCCAGAUACAGCAGAAUGCAGUU-3′ | 52.0 | 331 |
| | 3′-CCGGGUCUAUGUCGUCUUACGUCAA-5′ | | 332 |
| 339 | 5′-CCAGAUACAGCAGAAUGCAGUUCAG-3′ | 48.0 | 333 |
| | 3′-GGUCUAUGUCGUCUUACGUCAAGUC-5′ | | 334 |
| 341 | 5′-AGAUACAGCAGAAUGCAGUUCAGAA-3′ | 40.0 | 335 |
| | 3′-UCUAUGUCGUCUUACGUCAAGUCUU-5′ | | 336 |
| 351 | 5′-GAAUGCAGUUCAGAACCACACGGCU-3′ | 52.0 | 337 |
| | 3′-CUUACGUCAAGUCUUGGUGUGCCGA-5′ | | 338 |
| 453 | 5′-GGUACUAAAUCAAACUUCUCGACUU-3′ | 36.0 | 339 |
| | 3′-CCAUGAUUUAGUUUGAAGAGCUGAA-5′ | | 340 |
| 473 | 5′-GACUUGAGAUACAGCUGCUGGAGAA-3′ | 48.0 | 341 |
| | 3′-CUGAACUCUAUGUCGACGACCUCUU-5′ | | 342 |
| 651 | 5′-GAACCUUCAAGGCUUGGUUACUCGU-3′ | 48.0 | 343 |
| | 3′-CUUGGAAGUUCCGAACCAAUGAGCA-5′ | | 344 |
| 653 | 5′-ACCUUCAAGGCUUGGUUACUCGUCA-3′ | 48.0 | 345 |
| | 3′-UGGAAGUUCCGAACCAAUGAGCAGU-5′ | | 346 |
| 658 | 5′-CAAGGCUUGGUUACUCGUCAAACAU-3′ | 44.0 | 347 |
| | 3′-GUUCCGAACCAAUGAGCAGUUUGUA-5′ | | 348 |
| 660 | 5′-AGGCUUGGUUACUCGUCAAACAUAU-3′ | 40.0 | 349 |
| | 3′-UCCGAACCAAUGAGCAGUUUGUAUA-5′ | | 350 |
| 662 | 5′-GCUUGGUUACUCGUCAAACAUAUAU-3′ | 36.0 | 351 |
| | 3′-CGAACCAAUGAGCAGUUUGUAUAUA-5′ | | 352 |
| 764 | 5′-UGGACACAGUCCACAACCUUGUCAA-3′ | 48.0 | 353 |
| | 3′-ACCUGUGUCAGGUGUUGGAACAGUU-5′ | | 354 |
| 768 | 5′-CACAGUCCACAACCUUGUCAAUCUU-3′ | 44.0 | 355 |
| | 3′-GUGUCAGGUGUUGGAACAGUUAGAA-5′ | | 356 |
| 770 | 5′-CAGUCCACAACCUUGUCAAUCUUUG-3′ | 44.0 | 357 |
| | 3′-GUCAGGUGUUGGAACAGUUAGAAAC-5′ | | 358 |
| 774 | 5′-CCACAACCUUGUCAAUCUUUGCACU-3′ | 44.0 | 359 |
| | 3′-GGUGUUGGAACAGUUAGAAACGUGA-5′ | | 360 |
| 832 | 5′-GAAGAGAAACCAUUUAGAGACUGUG-3′ | 40.0 | 361 |
| | 3′-CUUCUCUUUGGUAAAUCUCUGACAC-5′ | | 362 |
| 840 | 5′-ACCAUUUAGAGACUGUGCAGAUGUA-3′ | 40.0 | 363 |
| | 3′-UGGUAAAUCUCUGACACGUCUACAU-5′ | | 364 |
| 846 | 5′-UAGAGACUGUGCAGAUGUAUAUCAA-3′ | 36.0 | 365 |
| | 3′-AUCUCUGACACGUCUACAUAUAGUU-5′ | | 366 |
| 991 | 5′-GAUGGAAGUCUAGAUUUCCAAAGAG-3′ | 40.0 | 367 |
| | 3′-CUACCUUCAGAUCUAAAGGUUUCUC-5′ | | 368 |
| 1098 | 5′-UCAGAGGCAGUACAUGCUAAGAAUU-3′ | 40.0 | 369 |
| | 3′-AGUCUCCGUCAUGUACGAUUCUUAA-5′ | | 370 |
| 1147 | 5′-CGAGCCUAUUCACAGUAUGACAGAU-3′ | 44.0 | 371 |
| | 3′-GCUCGGAUAAGUGUCAUACUGUCUA-5′ | | 372 |
| 1164 | 5′-UGACAGAUUCCACAUAGGAAAUGAA-3′ | 36.0 | 373 |
| | 3′-ACUGUCUAAGGUGUAUCCUUUACUU-5′ | | 374 |
| 1257 | 5′-ACACGGUGCUGAUUUCAGCACUAAA-3′ | 44.0 | 375 |
| | 3′-UGUGCCACGACUAAAGUCGUGAUUU-5′ | | 376 |
| 1258 | 5′-CACGGUGCUGAUUUCAGCACUAAAG-3′ | 48.0 | 377 |
| | 3′-GUGCCACGACUAAAGUCGUGAUUUC-5′ | | 378 |
| −1260 | 5′-CGGUGCUGAUUUCAGCACUAAAGAU-3′ | 44.0 | 379 |
| | 3′-GCCACGACUAAAGUCGUGAUUUCUA-5′ | | 380 |
| 1282 | 5′-GAUGCUGAUAAUGACAACUGUAUGU-3′ | 36.0 | 381 |
| | 3′-CUACGACUAUUACUGUUGACAUACA-5′ | | 382 |
| 1285 | 5′-GCUGAUAAUGACAACUGUAUGUGCA-3′ | 40.0 | 383 |
| | 3′-CGACUAUUACUGUUGACAUACACGU-5′ | | 384 |
| 1371 | 5′-UGGAAUGUUCUAUACUGCGGGACAA-3′ | 44.0 | 385 |
| | 3′-ACCUUACAAGAUAUGACGCCCUGUU-5′ | | 386 |
| 1409 | 5′-UGAAUGGGAUAAAGUGGCACUACUU-3′ | 40.0 | 387 |
| | 3′-ACUUACCCUAUUUCACCGUGAUGAA-5′ | | 388 |

TABLE 6
siRNA candidates for mouse ANGPT1.

| | siRNA Sequence | | SEQ |
|---|---|---|---|
| | (sense strand/ | | ID |
| Start | anti-sense strand) | GC % | NO: |
| 706 | 5′-CAACUUAGUAGAGCUACCAACAACA-3′ | 40.0 | 389 |
| | 3′-GUUGAAUCAUCUCGAUGGUUGUUGU-5′ | | 390 |
| 845 | 5′-CAUUUCGAGACUGUGCAGAUGUAUA-3′ | 40.0 | 391 |
| | 3′-GUAAAGCUCUGACACGUCUACAUAU-5′ | | 392 |
| 989 | 5′-GGGAAGAUGGAAGCCUGGAUUUCCA-3′ | 52.0 | 393 |
| | 3′-CCCUUCUACCUUCGGACCUAAAGGU-5′ | | 394 |
| 1052 | 5′-CCUCUGGUGAAUAUUGGCUCGGGAA-3′ | 52.0 | 395 |
| | 3′-GGAGACCACUUAUAACCGAGCCCUU-5′ | | 396 |
| 1119 | 5′-GAGGAUUGAGCUGAUGGACUGGGAA-3′ | 52.0 | 397 |
| | 3′-CUCCUAACUCGACUACCUGACCCUU-5′ | | 398 |
| 1167 | 5′-CGACAGAUUCCACAUAGGAAAUGAA-3′ | 40.0 | 399 |
| | 3′-GCUGUCUAAGGUGUAUCCUUUACUU-5′ | | 400 |
| 1238 | 5′-GCAAACAGAGCAGCUUGAUCUUACA-3′ | 44.0 | 401 |
| | 3′-CGUUUGUCUCGUCGAACUAGAAUGU-5′ | | 402 |
| 1248 | 5′-CAGCUUGAUCUUACACGGUGCUGAU-3′ | 48.0 | 403 |
| | 3′-GUCGAACUAGAAUGUGCCACGACUA-5′ | | 404 |
| 1360 | 5′-CCUUCCAAUCUAAAUGGAAUGUUCU-3′ | 36.0 | 405 |
| | 3′-GGAAGGUUAGAUUUACCUUACAAGA-5′ | | 406 |
| 1427 | 5′-GGCACUACUUCAAAGGGCCCAGUUA-3′ | 52.0 | 407 |
| | 3′-CCGUCAUGAAGUUUCCCGGGUCAAU-5′ | | 408 |
| 109 | 5′-CAACAUGGGCAAUGUGCCUACACUU-3′ | 48.0 | 409 |
| | 3′-GUUGUACCCGUUACACGGAUGUGAA-5′ | | 410 |
| 112 | 5′-CAUGGGCAAUGUGCCUACACUUUCA-3′ | 48.0 | 411 |
| | 3′-GUACCCGUUACACGGAUGUGAAAGU-5′ | | 412 |
| 125 | 5′-CCUACACUUUCAUUCUUCCAGAACA-3′ | 40.0 | 413 |
| | 3′-GGAUGUGAAAGUAAGAAGGUCUUGU-5′ | | 414 |
| 339 | 5′-CCAGAUACAACAGAAUGCUGUUCAA-3′ | 40.0 | 415 |
| | 3′-GGUCUAUGUUGUCUUACGACAAGUU-5′ | | 416 |
| 437 | 5′-CAGAUGUUGAGACCCAGGUACUAAA-3′ | 44.0 | 417 |
| | 3′-GUCUACAACUCUGGGUCCAUGAUUU-5′ | | 418 |
| 453 | 5′-GGUACUAAAUCAAACAUCCCGACUU-3′ | 40.0 | 416 |
| | 3′-CCAUGAUUUAGUUUGUAGGGCUGAA-5′ | | 420 |
| 467 | 5′-CAUCCCGACUUGAAAUACAACUGCU-3′ | 44.0 | 421 |
| | 3′-GUAGGGCUGAACUUUAUGUUGACGA-5′ | | 422 |
| 473 | 5′-GACUUGAAAUACAACUGCUAGAGAA-3′ | 36.0 | 423 |
| | 3′-CUGAACUUUAUGUUGACGAUCUCUU-5′ | | 424 |
| 509 | 5′-CAUACAAGCUAGAGAAGCAACUUCU-3′ | 40.0 | 425 |
| | 3′-GUAUGUUCGAUCUCUUCGUUGAAGA-5′ | | 426 |
| 525 | 5′-GCAACUUCUCCAACAGACAAAUGAA-3′ | 40.0 | 427 |
| | 3′-CGUUGAAGAGGUUGUCUGUUUACUU-5′ | | 428 |
| 755 | 5′-UGGAGCUCAUGGACACAGUUCAUAA-3′ | 44.0 | 429 |
| | 3′-ACCUCGAGUACCUGUGUCAAGUAUU-5′ | | 430 |
| 1162 | 5′-CAGUACGACAGAUUCCACAUAGGAA-3′ | 44.0 | 431 |
| | 3′-GUCAUGCUGUCUAAGGUGUAUCCUU-5′ | | 432 |

TABLE 7
siRNA candidates for human/mouse ANGPT1.

| | siRNA Sequence | | SEQ |
|---|---|---|---|
| | (sense strand/ | | ID |
| Start | anti-sense strand) | GC % | NO: |
| 109 | 5′-CAACAUGGGCAAUGUGCCUACACUU-3′ | 48.0 | 433 |
| | 3′-GUUGUACCCGUUACACGGAUGUGAA-5′ | | 434 |
| 112 | 5′-CAUGGGCAAUGUGCCUACACUUUCA-3′ | 48.0 | 435 |
| | 3′-GUACCCGUUACACGGAUGUGAAAGU-5′ | | 436 |
| 125 | 5′-CCUACACUUUCAUUCUUCCAGAACA-3′ | 40.0 | 437 |
| | 3′-GGAUGUGAAAGUAAGAAGGUCUUGU-5′ | | 438 |
| 89 | 5′-GGAGAAGAUAUAACCGGAUUCAACA-3′ | 40.0 | 439 |
| | 3′-CCUCUUCUAUAUUGGCCUAAGUUGU-5′ | | 440 |
| 95 | 5′-GAUAUAACCGGAUUCAACAUGGGCA-3′ | 44.0 | 441 |
| | 3′-CUAUAUUGGCCUAAGUUGUACCCGU-5′ | | 442 |
| 108 | 5′-UCAACAUGGGCAAUGUGCCUACACU-3′ | 48.0 | 443 |
| | 3′-AGUUGUACCCGUUACACGGAUGUGA-5′ | | 444 |
| 437 | 5′-CAGAUGUUGAGACCCAGGUACUAAA-3′ | 44.0 | 445 |
| | 3′-GUCUACAACUCUGGGUCCAUGAUUU-5′ | | 446 |
| 1168 | 5′-GACAGAUUCCACAUAGGAAAUGAAA-3′ | 36.0 | 447 |
| | 3′-CUGUCUAAGGUGUAUCCUUUACUUU-5′ | | 448 |
| 1409 | 5′-UGAAUGGGAUAAAGUGGCACUACUU-3′ | 40.0 | 449 |
| | 3′-ACUUACCCUAUUUCACCGUGAUGAA-5′ | | 450 |
| 1412 | 5′-UGAAUGGGAUAAAGUGGCACUACUU-3′ | 40.0 | 451 |
| | 3′-ACUUACCCUAUUUCACCGUGAUGAA-5′ | | 452 |
| 1427 | 5′-GGCACUACUUCAAAGGGCCCAGUUA-3′ | 52.0 | 453 |
| | 3′-CCGUGAUGAAGUUUCCCGGGUCAAU-5′ | | 454 |

TABLE 8
siRNA candidates for human ANGPT2.

| | siRNA Sequence | | SEQ |
|---|---|---|---|
| | (sense strand/ | | ID |
| Start | anti-sense strand) | GC % | NO: |
| 812 | 5′-CCACUGUUGCUAAAGAAGAACAAAU-3′ | 36.0 | 455 |
| | 3′-GGUGACAACGAUUUCUUCUUGUUUA-5′ | | 456 |
| 837 | 5′-CAGCUUCAGAGACUGUGCUGAAGUA-3′ | 48.0 | 457 |
| | 3′-GUCGAAGUCUCUGACACGACUUCAU-5′ | | 458 |
| 871 | 5′-GGACACACCACAAAUGGCAUCUACA-3′ | 48.0 | 459 |
| | 3′-CCUGUGUGGUGUUUACCGUAGAUGU-5′ | | 460 |
| 888 | 5′-CAUCUACACGUUAACAUUCCCUAAU-3′ | 36.0 | 461 |
| | 3′-GUAGAUGUGCAAUUGUAAGGGAUUA-5′ | | 462 |
| 951 | 5′-UGGAGGAGGCGGGUGGACAAUUAUU-3′ | 52.0 | 463 |
| | 3′-ACCUCCUCCGCCCACCUGUUAAUAA-5′ | | 464 |
| 962 | 5′-GGUGGACAAUUAUUCAGCGACGUGA-3′ | 48.0 | 465 |
| | 3′-CCACCUGUUAAUAAGUCGCUGCACU-5′ | | 466 |
| 1082 | 5′-CGCAACUGACUAAUCAGCAACGCUA-3′ | 48.0 | 467 |
| | 3′-GCGUUGACUGAUUAGUCGUUGCGAU-5′ | | 468 |
| 1242 | 5′-CAGCAUCAGCCAACCAGGAAAUGAU-3′ | 48.0 | 469 |
| | 3′-GUCGUAGUCGGUUGGUCCUUUACUA-5′ | | 470 |
| 1354 | 5′-CCUUCCAACUUGAACGGAAUGUACU-3′ | 44.0 | 471 |
| | 3′-GGAAGGUUGAACUUGCCUUACAUGA-5′ | | 472 |
| 1390 | 5′-CAGAACACAAAUAAGUUCAACGGCA-3′ | 40.0 | 473 |
| | 3′-GUCUUGUGUUUAUUCAAGUUGCCGU-5′ | | 474 |
| 34 | 5′-GAUCUUGUCUUGGCCGCAGCCUAUA-3′ | 52.0 | 475 |
| | 3′-CUAGAACAGAACCGGCGUCGGAUAU-5′ | | 476 |
| 47 | 5′-CCGCAGCCUAUAACAACUUUCGGAA-3′ | 48.0 | 477 |
| | 3′-GGCGUCGGAUAUUGUUGAAAGCCUU-5′ | | 478 |
| 241 | 5′-CAAGUGCUGGAGAACAUCAUGGAAA-3′ | 44.0 | 479 |
| | 3′-GUUCACGACCUCUUGUAGUACCUUU-5′ | | 480 |
| 306 | 5′-GGACAACAUGAAGAAAGAAAUGGUA-3′ | 36.0 | 481 |
| | 3′-CCUGUUGUACUUCUUUCUUUACCAU-5′ | | 482 |
| 390 | 5′-CCUGUUGAACCAAACAGCUGAGCAA-3′ | 48.0 | 483 |
| | 3′-GGACAACUUGGUUUGUCGACUCGUU-5′ | | 484 |
| 425 | 5′-UAACUGAUGUGGAAGCCCAAGUAUU-3′ | 40.0 | 485 |
| | 3′-AUUGACUACACCUUCGGGUUCAUAA-5′ | | 486 |
| 458 | 5′-CCACGAGACUUGAACUUCAGCUCUU-3′ | 48.0 | 487 |
| | 3′-GGUGCUCUGAACUUGAAGUCGAGAA-5′ | | 488 |
| 877 | 5′-ACCACAAAUGGCAUCUACACGUUAA-3′ | 40.0 | 489 |
| | 3′-UGGUGUUUACCGUAGAUGUGCAAUU-5′ | | 490 |
| 894 | 5′-CACGUUAACAUUCCCUAAUUCUACA-3′ | 36.0 | 491 |
| | 3′-GUGCAAUUGUAAGGGAUUAAGAUGU-5′ | | 492 |
| 1032 | 5′-GGGAUUUGGUAACCCUUCAGGAGAA-3′ | 48.0 | 493 |
| | 3′-CCCUAAACCAUUGGGAAGUCCUCUU-5′ | | 494 |
| 1342 | 5′-GAUGCAUGUGGUCCUUCCAACUUGA-3′ | 48.0 | 495 |
| | 3′-CUACGUACACCAGGAAGGUUGAACU-5′ | | 496 |
| 1410 | 5′-CGGCAUUAAAUGGUACUACUGGAAA-3′ | 40.0 | 497 |
| | 3′-GCCGUAAUUUACCAUGAUGACCUUU-5′ | | 498 |
| −59 | 5′-UCUGGACGUGUGUUUGCCCUCAAGU-3′ | 52.0 | 499 |
| | 3′-AGACCUGCACACAAACGGGAGUUCA-5′ | | 500 |
| −57 | 5′-UGGACGUGUGUUUGCCCUCAAGUUU-3′ | 48.0 | 501 |
| | 3′-ACCUGCACACAAACGGGAGUUCAAA-5′ | | 502 |
| −56 | 5′-GGACGUGUGUUUGCCCUCAAGUUUG-3′ | 52.0 | 503 |
| | 3′-CCUGUAUAUAAACGGGAGUUCAAAC-5′ | | 504 |
| −13 | 5′-ACUGAAGAAAGAAUGUGGCAGAUUG-3′ | 40.0 | 505 |
| | 3′-UGACUUCUUUCUUACACCGUCUAAC-5′ | | 506 |
| −10 | 5′-GAAGAAAGAAUGUGGCAGAUUGUUU-3′ | 36.0 | 507 |
| | 3′-CUUCUUUCUUACACCGUCUAACAAA-5′ | | 508 |
| 33 | 5′-UGAUCUUGUCUUGGCCGCAGCCUAU-3′ | 52.0 | 509 |
| | 3′-ACUAGAACAGAACCGGCGUCGGAUA-5′ | | 510 |
| 46 | 5′-GCCGCAGCCUAUAACAACUUUCGGA-3′ | 52.0 | 511 |
| | 3′-CGGCGUCGGAUAUUGUUGAAAGCCU-5′ | | 512 |
| 53 | 5′-CCUAUAACAACUUUCGGAAGAGCAU-3′ | 40.0 | 513 |
| | 3′-GGAUAUUGUUGAAAGCCUUCUCGUA-5′ | | 514 |
| 274 | 5′-CAGUGGCUAAUGAAGCUUGAGAAUU-3′ | 40.0 | 515 |
| | 3′-GUCACCGAUUACUUCGAACUCUUAA-5′ | | 516 |
| 275 | 5′-AGUGGCUAAUGAAGCUUGAGAAUUA-3′ | 36.0 | 517 |
| | 3′-UCACCGAUUACUUCGAACUCUUAAU-5′ | | 518 |
| 355 | 5′-AACCAGACGGCUGUGAUGAUAGAAA-3′ | 44.0 | 519 |
| | 3′-UUGGUCUGCCGACACUACUAUCUUU-5′ | | 520 |
| 357 | 5′-CCAGACGGCUGUGAUGAUAGAAAUA-3′ | 44.0 | 521 |
| | 3′-GGUCUGCCGACACUACUAUCUUUAU-5′ | | 522 |
| 403 | 5′-ACAGCUGAGCAAACGCGGAAGUUAA-3′ | 48.0 | 523 |
| | 3′-UGUCGACUCGUUUGCGCCUUCAAUU-5′ | | 524 |
| 414 | 5′-AACGCGGAAGUUAACUGAUGUGGAA-3′ | 44.0 | 525 |
| | 3′-UUGCGCCUUCAAUUGACUACACCUU-5′ | | 526 |
| 419 | 5′-GGAAGUUAACUGAUGUGGAAGCCCA-3′ | 48.0 | 527 |
| | 3′-CCUUCAAUUGACUACACCUUCGGGU-5′ | | 528 |
| 420 | 5′-GAAGUUAACUGAUGUGGAAGCCCAA-3′ | 44.0 | 529 |
| | 3′-CUUCAAUUGACUACACCUUCGGGUU-5′ | | 530 |
| 427 | 5′-ACUGAUGUGGAAGCCCAAGUAUUAA-3′ | 40.0 | 531 |
| | 3′-UGACUACACCUUCGGGUUCAUAAUU-5′ | | 532 |
| 444 | 5′-AGUAUUAAAUCAGACCACGAGACUU-3′ | 36.0 | 533 |
| | 3′-UCAUAAUUUAGUCUGGUGCUCUGAA-5′ | | 534 |
| 483 | 5′-GGAACACUCCCUCUCGACAAACAAA-3′ | 48.0 | 535 |
| | 3′-CCUUGUGAGGGAGAGCUGUUUGUUU-5′ | | 536 |
| 524 | 5′-UGGACCAGACCAGUGAAAUAAACAA-3′ | 40.0 | 537 |
| | 3′-ACCUGGUCUGGUCACUUUAUUUGUU-5′ | | 538 |
| 811 | 5′-CCCACUGUUGCUAAAGAAGAACAAA-3′ | 40.0 | 539 |
| | 3′-GGGUGACAACGAUUUCUUCUUGUUU-5′ | | 540 |
| 820 | 5′-GCUAAAGAAGAACAAAUCAGCUUCA-3′ | 36.0 | 541 |
| | 3′-CGAUUUCUUCUUGUUUAGUCGAAGU-5′ | | 542 |
| 876 | 5′-CACCACAAAUGGCAUCUACACGUUA-3′ | 44.0 | 543 |
| | 3′-GUGGUGUUUACCGUAGAUGUGCAAU-5′ | | 544 |
| 881 | 5′-CAAAUGGCAUCUACACGUUAACAUU-3′ | 36.0 | 545 |
| | 3′-GUUUACCGUAGAUGUGCAAUUGUAA-5′ | | 546 |
| 924 | 5′-GAUCAAGGCCUACUGUGACAUGGAA-3′ | 48.0 | 547 |
| | 3′-CUAGUUCCGGAUGACACUGUACCUU-5′ | | 548 |
| 953 | 5′-GAGGAGGCGGGUGGACAAUUAUUCA-3′ | 52.0 | 549 |
| | 3′-CUCCUCCGCCCACCUGUUAAUAAGU-5′ | | 550 |
| 980 | 5′-GACGUGAGGAUGGCAGCGUUGAUUU-3′ | 52.0 | 551 |
| | 3′-CUGCACUCCUACCGUCGCAACUAAA-5′ | | 552 |
| 1066 | 5′-GGAAAUGAGUUUGUUUCGCAACUGA-3′ | 40.0 | 553 |
| | 3′-CCUUUACUCAAACAAAGCGUUGACU-5′ | | 554 |
| 1067 | 5′-GAAAUGAGUUUGUUUCGCAACUGAC-3′ | 40.0 | 555 |
| | 3′-CUUUACUCAAACAAAGCGUUGACUG-5′ | | 556 |
| 1140 | 5′-GAAUGAGGCUUACUCAUUGUAUGAA-3′ | 36.0 | 557 |
| | 3′-CUUACUCCGAAUGAGUAACAUACUU-5′ | | 558 |
| 1144 | 5′-GAGGCUUACUCAUUGUAUGAACAUU-3′ | 36.0 | 559 |
| | 3′-CUCCGAAUGAGUAACAUACUUGUAA-5′ | | 560 |
| 1273 | 5′-ACAAAGGAUGGAGACAACGACAAAU-3′ | 40.0 | 561 |
| | 3′-UGUUUCCUACCUCUGUUGCUGUUUA-5′ | | 562 |
| 1277 | 5′-AGGAUGGAGACAACGACAAAUGUAU-3′ | 40.0 | 563 |
| | 3′-UCCUACCUCUGUUGCUGUUUACAUA-5′ | | 564 |
| 1283 | 5′-GAGACAACGACAAAUGUAUUUGCAA-3′ | 36.0 | 565 |
| | 3′-CUCUGUUGCUGUUUACAUAAACGUU-5′ | | 566 |
| 1359 | 5′-CAACUUGAACGGAAUGUACUAUCCA-3′ | 40.0 | 567 |
| | 3′-GUUGAACUUGCCUUACAUGAUAGGU-5′ | | 568 |
| 1392 | 5′-GAACACAAAUAAGUUCAACGGCAUU-3′ | 36.0 | 589 |
| | 3′-CUUGUGUUUAUUCAAGUUGCCGUAA-5′ | | 590 |
| 1421 | 5′-GGUACUACUGGAAAGGCUCAGGCUA-3′ | 52.0 | 591 |
| | 3′-CCAUGAUGACCUUUCCGAGUCCGAU-5′ | | 592 |
| 1423 | 5′-UACUACUGGAAAGGCUCAGGCUAUU-3′ | 44.0 | 593 |
| | 3′-AUGAUGACCUUUCCGAGUCCGAUAA-5′ | | 594 |
| 1429 | 5′-UGGAAAGGCUCAGGCUAUUCGCUCA-3′ | 52.0 | 595 |
| | 3′-ACCUUUCCGAGUCCGAUAAGCGAGU-5′ | | 596 |
| 1458 | 5′-CACAACCAUGAUGAUCCGACCAGCA-3′ | 52.0 | 597 |
| | 3′-GUGUUGGUACUACUAGGCUGGUCGU-5′ | | 598 |
| 1533 | 5′-AAGACUUAAGCCCAGUGCACUGAAA-3′ | 44.0 | 599 |
| | 3′-UUCUGAAUUCGGGUCACGUGACUUU-5′ | | 600 |
| 1620 | 5′-CCACAUGCUCCAGAUUAGAGCCUGU-3′ | 52.0 | 601 |
| | 3′-GGUGUACGAGGUCUAAUCUCGGACA-5′ | | 602 |
| 1621 | 5′-CACAUGCUCCAGAUUAGAGCCUGUA-3′ | 48.0 | 603 |
| | 3′-GUGUACGAGGUCUAAUCUCGGACAU-5′ | | 604 |
| 1623 | 5′-CAUGCUCCAGAUUAGAGCCUGUAAA-3′ | 44.0 | 605 |
| | 3′-GUACGAGGUCUAAUCUCGGACAUUU-5′ | | 606 |
| 1628 | 5′-UCCAGAUUAGAGCCUGUAAACUUUA-3′ | 36.0 | 607 |
| | 3′-AGGUCUAAUCUCGGACAUUUGAAAU-5′ | | 608 |

TABLE 9
siRNA candidates for mouse ANGPT2.

| | siRNA Sequence | | SEQ |
|---|---|---|---|
| | (sense strand/ | | ID |
| Start | anti-sense strand) | GC % | NO: |
| 474 | 5′-GCAGCUUCUCCAACAUUCUAUUUCU-3′ | 40.0 | 609 |
| | 3′-CGUCGAAGAGGUUGUAAGAUAAAGA-5′ | | 610 |
| 713 | 5′-CGGUCAACAACUCGCUCCUUCAGAA-3′ | 52.0 | 611 |
| | 3′-GCCAGUUGUUGAGCGAGGAAGUCUU-5′ | | 612 |
| 761 | 5′-CCGUCAACAGCUUGCUGACCAUGAU-3′ | 52.0 | 613 |
| | 3′-GGCAGUUGUCGAACGACUGGUACUA-5′ | | 614 |
| 983 | 5′-GAGAAGAUGGCAGUGUGGACUUCCA-3′ | 52.0 | 615 |
| | 3′-CUCUUCUACCGUCACACCUGAAGGU-5′ | | 616 |
| 1066 | 5′-GGCAAUGAGUUUGUCUCCCAGCUGA-3′ | 52.0 | 617 |
| | 3′-CCGUUACUCAAACAGAGGGUCGACU-5′ | | 618 |
| 1103 | 5′-GCUACGUGCUUAAGAUCCAGCUGAA-3′ | 48.0 | 619 |
| | 3′-CGAUGCACGAAUUCUAGGUCGACUU-5′ | | 620 |
| 1148 | 3′-GCGUAAGCGACAUACUAGUGAAGAU-5′ | 44.0 | 621 |
| | 5′-CGCAUUCGCUGUAUGAUCACUUCUA-3′ | | 622 |
| 1242 | 5′-UAGCAUCAGCCAACCAGGAAGUGAU-3′ | 48.0 | 623 |
| | 3′-AUCGUAGUCGGUUGGUCCUUCACUA-5′ | | 624 |
| 1288 | 5′-AAUGACAAAUGCAUCUGCAAGUGUU-3′ | 36.0 | 625 |
| | 3′-UUACUGUUUACGUAGACGUUCACAA-5′ | | 626 |
| 1354 | 5′-CCUUCCAACUUGAAUGGACAGUACU-3′ | 44.0 | 627 |
| | 3′-GGAAGGUUGAACUUACCUGUCAUGA-5′ | | 628 |
| 475 | 5′-CAGCUUCUCCAACAUUCUAUUUCUA-3′ | 36.0 | 629 |
| | 3′-GUCGAAGAGGUUGUAAGAUAAAGAU-5′ | | 630 |
| 742 | 5′-CAGCAUGACCUAAUGGAGACCGUCA-3′ | 52.0 | 631 |
| | 3′-GUCGUACUGGAUUACCUCUGGCAGU-5′ | | 632 |
| 801 | 5′-CAAGAGCUCGGUUGCUAUCCGUAAA-3′ | 48.0 | 633 |
| | 3′-GUUCUCGAGCCAACGAUAGGCAUUU-5′ | | 634 |
| 1342 | 5′-GACGCAUGUGGUCCUUCCAACUUGA-3′ | 52.0 | 635 |
| | 3′-CUGCGUACACCAGGAAGGUUGAACU-5′ | | 636 |

TABLE 10
siRNA candidates for human/mouse ANGPT-2.

| | siRNA Sequence | | SEQ |
|---|---|---|---|
| | (sense strand/ | | ID |
| Start | anti-sense strand) | GC % | NO: |
| 922 | 5′-GAGAUCAAGGCCUACUGUGACAUGG-3′ | 52.0 | 637 |
| | 3′-CUCUAGUUCCGGAUGACACUGUACC-5′ | | 638 |
| 923 | 5′-AGAUCAAGGCCUACUGUGACAUGGA-3′ | 48.0 | 639 |
| | 3′-UCUAGUUCCGGAUGACACUGUACCU-5′ | | 640 |
| 1447 | 5′-UCGCUCAAGGCCACAACCAUGAUGA-3′ | 52.0 | 641 |
| | 3′-AGCGAGUUCCGGUGUUGGUACUACU-5′ | | 642 |
| 1448 | 5′-CGCUCAAGGCCACAACCAUGAUGAU-3′ | 52.0 | 643 |
| | 3′-GCGAGUUCCGGUGUUGGUACUACUA-5′ | | 644 |
| 1449 | 5′-GCUCAAGGCCACAACCAUGAUGAUC-3′ | 52.0 | 645 |
| | 3′-CGAGUUCCGGUGUUGGUACUACUAG-5′ | | 646 |
| 1450 | 5′-CUCAAGGCCACAACCAUGAUGAUCC-3′ | 52.0 | 647 |
| | 3′-GAGUUCCGGUGUUGGUACUACUAGG-5′ | | 648 |

The present invention provides methods for inhibition of individual or combinations of genes active in the Ang-Tie pathway. In some embodiments, the present invention provides a method of inhibiting or reducing angiogenesis in a tissue associated with undesired angiogenesis comprising administering to the tissue siRNA molecules that target Tie2 so that expression of Tie2 is decreased. In some embodiments, the present invention provides a method of inhibiting or reducing angiogenesis in a tissue associated with undesired angiogenesis comprising administering to the tissue siRNA molecules that target Ang-1 so that expression of Ang-1 is decreased. In further embodiments, the invention provides a method of inhibiting or reducing angiogenesis in a tissue associated with undesired angiogenesis comprising administering to the tissue siRNA molecules that target Ang-2 so that expression of Ang-2 is decreased. In one embodiment, the tissue is a tumor.

### Combined Ang/Tie2 Pathway Gene Inhibition

The compositions and methods of the present invention for inhibition of angiogenesis are based on several fundamental aspects. First, pathological angiogenesis is a complex process and results from interactions of multiple proteins which are abnormally expressed or over-expressed in diseased tissues. Second, nucleic acid agents that activate RNAi are highly selective in a sequence specific manner. Third, inhibition of angiogenesis by modulation of protein activity can be operative by many methods, including but not limited to an inhibition of protein function (antagonists), stimulation of protein function (agonists), reduction of protein expression levels, and post transcriptional modification of proteins. Importantly, it may be desirable in the treatment of disease to effectively shut down a particular biological pathway that is critical for disease progression, by simultaneously blocking functions of ligands and their receptors, simultaneously blocking receptor activity and the activity of down stream signaling proteins, and/or simultaneously blocking redundant elements of a pathway. Such methods may be used for treating angiogenesis-related diseases including those that involve the Ang/Tie2 pathway.

Although clinical studies have demonstrated remarkable therapeutic efficacies, the toxicities of higher dosage and long term safety are major concerns, due to the different origins, different manufacturing processes and different chemistry properties of the components.

To overcome these problems, aspects of the present invention provide compositions of and methods of using nucleic acid molecules, including siRNA oligonucleotides, to provide a unique advantage, i.e., to achieve combinatorial effects with a combination of nucleic acid molecules, including siRNAs, that target multiple disease causing genes or target different sequences in the same gene in the same treatment. One advantage of the compositions and methods of the present invention is that all siRNA oligonucleotides are very similar chemically, pharmacologically, and can be produced from the same source and using the same manufacturing process. Another advantage provided by the present invention is that multiple siRNA oligonucleotides can be formulated in a single preparation such as a nanoparticle preparation.

Therefore, an aspect of the present invention is to combine nucleic acid molecules, including siRNAs, so as to achieve specific and selective silencing of multiple genes in the Ang/Tie2 pathway and as a result achieve an inhibition of angiogenesis-related disease and a better clinical benefit. The present invention provides for combinations of siRNA targets including combinations of two or more targets selected from: Tie2, Ang-1 and Ang-2. The present invention also provides for combinations of siRNAs targeting one or more sequences within the same gene in the Ang/Tie2 pathway. Exemplary siRNA sequences silencing these mRNAs are listed in Tables 2-10. Such siRNA compositions may also be combined with siRNA that targets other angiogenic pathways such as the VEGF pathway, PDGF and EGF and their receptors, downstream signaling factors including RAF and AKT, and transcription factors including NFκB. Such siRNA compositions may also be combined with siRNA that target genes downstream of Tie2, Ang-1 and Ang-2.

In one embodiment a combination of siRNA inhibiting Tie2 and two of its ligands Ang-1 and Ang-2 is used. In some embodiments, a combination of siRNA molecules that target Tie2 and siRNA molecules that target Ang-1 is used so that expression of both Tie2 and Ang-1 is decreased. In some embodiments, a combination of siRNA molecules that target Tie2 and siRNA molecules that target Ang-2 is used so that expression of both Tie2 and Ang-2 is decreased. In some embodiments, a combination of siRNA molecules that target Ang-1 and siRNA molecules that target Ang-2 is used so that expression of both Ang-1 and Ang-2 is decreased.

In some embodiments, the present invention provides a method of inhibiting or reducing angiogenesis in a tissue associated with undesired angiogenesis comprising administering to the tissue siRNA molecules that target Tie2 and siRNA molecules that target Ang-1 so that expression of Tie2 and Ang-1 is decreased. In some embodiments, the present invention provides a method of inhibiting or reducing angiogenesis in a tissue associated with undesired angiogenesis comprising administering to the tissue siRNA molecules that target Tie2 and siRNA molecules that target Ang-2 so that expression of Tie2 and Ang-2 is decreased. In some embodiments, the present invention provides a method of inhibiting or reducing angiogenesis in a tissue associated with undesired angiogenesis comprising administering to the tissue siRNA molecules that target Ang-1 and siRNA molecules that target Ang-2 so that expression of Ang-1 and Ang-2 is decreased. In further embodiments, the present invention provides a method of inhibiting or reducing angiogenesis in a tissue associated with undesired angiogenesis comprising administering to the tissue siRNA molecules that target Tie2, siRNA molecules that target Ang-1 and siRNA molecules that target Ang-2 so that expression of Tie2, Ang-1 and Ang-2 is decreased. In one embodiment, the tissue is a tumor.

Another embodiment of the invention is a combination of siRNA inhibiting Tie2, Ang-1 and Ang-2, PDGF and its receptors, and EGF and its receptors. Yet another embodiment is a combination of siRNA inhibiting the Tie2, Ang-1, and Ang-2 genes and their downstream signaling genes.

The siRNA oligonucleotides can be combined as a therapeutic for the treatment of angiogenesis-related disease. In one embodiment of the present invention they can be mixed together as a cocktail and in another embodiment they can be administered sequentially by the same route or by different routes and formulations and in yet another embodiment some can be administered as a cocktail and some administered sequentially. Other combinations of siRNA and methods for their combination will be understood by one skilled in the art to achieve treatment of angiogenesis-related diseases.

### Therapeutic Methods of Use

The present invention also provides methods for the treatment of angiogenesis-related diseases and conditions in a subject. In some embodiments, the present invention provides a method of treating a subject afflicted with a disease or condition associated with undesired angiogenesis comprising administering to the subject siRNA molecules that target Tie2 so that expression of Tie2 is decreased. In some embodiments, the present invention provides a method of treating a subject afflicted with a disease or condition associated with undesired angiogenesis comprising administering to the subject siRNA molecules that target Ang-1 so that expression of Ang-1 is decreased. In further embodiments, the present invention provides a method of treating a subject afflicted with a disease or condition associated with undesired angiogenesis comprising administering to the subject siRNA molecules that target Ang-2 so that expression of Ang-2 is decreased.

In some embodiments, the present invention provides a method of treating a subject afflicted with a disease or condition associated with undesired angiogenesis comprising administering to the subject siRNA molecules that target Tie2 and siRNA molecules that target Ang-1 so that expression of Tie2 and Ang-1 is decreased. In some embodiments, the present invention provides a method of treating a subject afflicted with a disease or condition associated with undesired angiogenesis comprising administering to the subject siRNA molecules that target Tie2 and siRNA molecules that target Ang-2 so that expression of Tie2 and Ang-2 is decreased. In some embodiments, the present invention provides a method of treating a subject afflicted with a disease or condition associated with undesired angiogenesis comprising administering to the subject siRNA molecules that target Ang-1 and siRNA molecules that target Ang-2 so that expression of Ang-1 and Ang-2 is decreased. In further embodiments, the present invention provides a method of treating a subject afflicted with a disease or condition associated with undesired angiogenesis comprising administering to the subject siRNA molecules that target Tie2, siRNA molecules that target Ang-1 and siRNA molecules that target Ang-2 so that expression of Tie2, Ang-1 and Ang-2 is decreased.

The present invention also provides methods for the treatment of angiogenesis-related disease in a subject, including cancer, ocular disease, arthritis, and inflammatory diseases. The angiogenesis-related diseases include, but are not limited to, carcinoma, such as breast, ovarian, stomach, endometrial, salivary gland, lung, kidney, colon, colorectum, esophageal, thyroid, pancreatic, prostate and bladder carcinomas and other neoplastic diseases, such as melanoma, small cell lung cancer, non-small cell lung cancer, glioma, hepatocellular (liver) carcinoma, sarcoma, head and neck cancers, mesothelioma, biliary (cholangiocarcinoma), small bowel adenocarcinoma, pediatric malignancies and glioblastoma.

Antagonizing these molecules is expected to inhibit pathophysiological processes, and thereby act as a potent therapy for various angiogenesis-dependent diseases. Besides solid tumors and their metastases, haematologic malignancies, such as leukemias, lymphomas and multiple myeloma, are also angiogenesis-dependent. Excessive vascular growth contributes to numerous non-neoplastic disorders. These non-neoplastic angiogenesis-dependent diseases include: atherosclerosis, haemangioma, haemangioendothelioma, angiofibroma, vascular malformations (e.g. Hereditary Hemorrhagic Teleangiectasia (HHT), or Osler-Weber syndrome), warts, pyogenic granulomas, excessive hair growth, Kaposis' sarcoma, scar keloids, allergic oedema, psoriasis, dysfunctional uterine bleeding, follicular cysts, ovarian hyperstimulation, endometriosis, respiratory distress, ascites, peritoneal sclerosis in dialysis patients, adhesion formation result from abdominal surgery, obesity, rheumatoid arthritis, synovitis, osteomyelitis, pannus growth, osteophyte, hemophilic joints, inflammatory and infectious processes (e.g. hepatitis, pneumonia, glomerulonephritis), asthma, nasal polyps, liver regeneration, pulmonary hypertension, retinopathy of prematurity, diabetic retinopathy, age-related macular degeneration, leukomalacia, neovascular glaucoma, corneal graft neovascularization, trachoma, thyroiditis, thyroid enlargement, and lymphoproliferative disorders.

In one embodiment of the invention, the subject treated is a human.

### Compositions and Methods of Administration

In another aspect, this invention provides compositions comprising the nucleic acid molecules, including siRNA, of the invention. The siRNA of the composition may be targeted to mRNA from the Ang-Tie pathway. The compositions may comprise the nucleic acid molecules and a pharmaceutically acceptable carrier, for example, a saline solution or a buffered saline solution.

In certain embodiments, this invention provides “naked” nucleic acid molecules or nucleic acid molecules in a vehicle which can be a naturally occurring or synthetic vector, such as a viral vector, a liposome, polylysine, or a cationic polymer. In one embodiment, the composition may comprise the siRNA of the invention and a complex-forming agent, such as a cationic polymer. The cationic polymer may be a histidine-lysine (HK) copolymer or a polyethyleneimine.

In certain embodiments, the cationic polymer is an HK copolymer. This HK copolymer is a copolymer of histidine and lysine. In certain embodiments, the HK copolymer is synthesized from any appropriate combination of polyhistidine, polylysine, histidine and/or lysine. In certain embodiments, the HK copolymer is linear. In certain preferred embodiments, the HK copolymer is branched.

In certain preferred embodiments, the branched HK copolymer comprises a polypeptide backbone. Preferably, the polypeptide backbone comprises 1-10 amino acid residues, and more preferably 2-5 amino acid residues.

In certain preferred embodiments, the polypeptide backbone consists of lysine amino acid residues.

In certain preferred embodiments, the number of branches on the branched HK copolymer is one greater than the number of backbone amino acid residues. In certain preferred embodiments, the branched HK copolymer contains 1-11 branches. In certain more preferred embodiments, the branched HK copolymer contains 2-5 branches. In certain even more preferred embodiments, the branched HK copolymer contains 4 branches.

In some embodiments, the branch of the branched HK copolymer comprises 10-100 amino acid residues. In certain preferred embodiments, the branch comprises 10-50 amino acid residues. In certain more preferred embodiments, the branch comprises 15-25 amino acid residues. In certain embodiments, the branch of the branched HK copolymer comprises at least 3 histidine amino acid residues in every subsegment of 5 amino acid residues. In certain other embodiments, the branch comprises at least 3 histidine amino acid residues in every subsegment of 4 amino acid residues. In certain other embodiments, the branch comprises at least 2 histidine amino acid residues in every subsegment of 3 amino acid residues. In certain other embodiments, the branch comprises at least 1 histidine amino acid residues in every subsegment of 2 amino acid residues.

In certain embodiments, at least 50% of the branch of the HK copolymer comprises units of the sequence KHHH. In certain preferred embodiments, at least 75% of the branch comprises units of the sequence KHHH.

In certain embodiments, the HK copolymer branch comprises an amino acid residue other than histidine or lysine. In certain preferred embodiments, the branch comprises a cysteine amino acid residue, wherein the cysteine is a N-terminal amino acid residue.

In certain embodiments, the HK copolymer has the structure (KHHHKHHHKHHHHKHHHK)₄-KKK. In certain other embodiments, the HK copolymer has the structure (CKHHHKHHHKHHHHKHHHK)₄-KKK.

Some suitable examples of HK copolymers can be found, for example, in U.S. Pat. Nos. 6,692,911 and 7,163,695, which are both incorporated herein by reference.

In one embodiment, the compositions of the invention may comprise the siRNA of the invention and a complex-forming agent that is used to make a nanoparticle. The nanoparticle may optionally comprise a steric polymer and/or a targeting moiety. The targeting moiety may be a peptide, an antibody, or an antigen-binding portion. The targeting moiety may serve as a means for targeting vascular endothelial cells, such as a peptide comprising the sequence Arg-Gly-Asp (RGD). Such a peptide may be cyclic or linear. In one embodiment, this peptide is RGDFK. In a certain embodiment, this peptide is cyclo (RGD-D-FK).

The nucleic acid molecules, compositions, and therapeutic methods of the invention can be used alone or in combination with other therapeutic agents and modalities including targeted therapeutics and including Ang-Tie pathway antagonists, such as monoclonal antibodies and small molecule inhibitors, and targeted therapeutics inhibiting EGF and its receptor, PDGF and its receptors, or MEK or Bcr-Abl, and other immunotherapeutic and chemotherapeutic agents, such as EGFR inhibitors VECTIBIX® (panitumumab) and TARCEVA® (erlotinib), Her-2-targeted therapy HERCEPTIN® (trastuzumab), or anti-angiogenesis drugs such as AVASTIN® (bevacizumab) and SUTENT® (sunitinib malate). The nucleic acid molecules, compositions, and methods also may be combined therapeutically with other treatment modalities including radiation, laser therapy, surgery and the like.

Methods of administration for the nucleic acids and compositions of the invention are known to those of ordinary skill in the art. Administration may be intravenous, intraperitoneal, intramuscular, intracavity, subcutaneous, cutaneous, or transdermal. In one embodiment, administration may be systemic. In a further embodiment, administration may be local. For example, the nucleic acid molecules of the invention may be delivered via direct injections into tumor tissue and directly into or near angiogenic tissue or tissue with undesirable neovasculature. For certain applications, the nucleic acid molecules and compositions may be administered with application of an electric field. In certain embodiments, this invention provides for administration of “naked” siRNA.

### Preparation of Nanoparticles Containing Nucleic Acid Molecules Modulating Expression of Ang/Tie2 Pathway Genes

One embodiment of the present invention provides compositions and methods for nanoparticle preparations of anti-Ang/Tie2 pathway nucleic acid molecules, including siRNAs. The nanoparticles may comprise one or more of a histidine-lysine copolymer, polyethylene glycol, or polyethyleneimine. In one embodiment of the invention, RGD-mediated ligand-directed nanoparticles may be prepared. In one method for the manufacture of RGD-mediated tissue-targeted nanoparticles containing siRNA, the targeting ligand, an RGD-containing peptide, is conjugated to a steric polymer such as polyethylene glycol, or other polymers with similar properties. This ligand-steric polymer conjugate is further conjugated to a polycation such as polyethyleneimine or other effective material such as a histidine-lysine copolymer. The conjugation can be by covalent or non-covalent bonds and the covalent bonds can be non-cleavable or they can be cleavable such as by hydrolysis or by reducing agents. A solution comprising the polymer conjugate, or comprising a mixture of a polymer conjugate with other polymer, lipid, or micelle such as materials comprising a ligand or a steric polymer or fusogen, is mixed with a solution comprising the nucleic acid, in one embodiment an siRNA targeted against specific mRNA of interest, in desirable ratios to obtain nanoparticles that contain siRNA. Such ratios may produce nanoparticles of a desired size, stability, or other characteristics.

In one embodiment, nanoparticles are formed by layered nanoparticle self-assembly comprising mixing the polymer conjugate with excess polycation and the nucleic acid. Non-covalent electrostatic interactions between the negatively charged nucleic acid and the positively charged segment of the polymer conjugate drive the self-assembly process that leads to formation of nanoparticles. This process involves simple mixing of the solutions where one of the solutions containing the nucleic acid is added to another solution containing the polymer conjugate and excess polycation followed by or concurrently with stirring. In one embodiment, the ratio between the positively charged components and the negatively charged components in the mixture is determined by appropriately adjusting the concentrations of each solution or by adjusting the volume of solution added. In another embodiment, the two solutions are mixed under continuous flow conditions using mixing apparatus such as static mixer. In this embodiment, two or more solutions are introduced into a static mixer at rates and pressures giving a ratio of the solutions, where the streams of solutions get mixed within the static mixer. Arrangements are possible for mixers to be arranged in parallel or in series.

The present invention, thus generally described, will be understood more readily by reference to the following examples, which are provided by way of illustration and are not intended to be limiting of the present invention. The invention is illustrated by the following examples but one skilled in the art will appreciate that the invention is not limited.

### Examples

### Example 1

### Selection of 48 Human Ang-2 siRNA Candidates for Potency Screening

To select potent human Ang-2 siRNA, 48 siRNA candidates were selected from Table 8 and Table 10 (Table 11). These siRNA were synthesized in plate-format at 20 nmol scale and used for in vitro potency screening.

TABLE 11
Human Ang-2 siRNA candidates for in vitro
screening

| | | siRNA Sequence | | SEQ |
|---|---|---|---|---|
| | | (sense strand/ | | ID |
| No. | Start | antisense strand) | GC % | NO: |
| 1 | −56 | 5′-GGACGUGUGUUUGCCCUCAAGUUUG-3′ | 52.0 | 503 |
| | | 3′-CCUGUAUAUAAACGGGAGUUCAAAC-5′ | | 504 |
| 2 | 34 | 5′-GAUCUUGUCUUGGCCGCAGCCUAUA-3′ | 52.0 | 475 |
| | | 3′-CUAGAACAGAACCGGCGUCGGAUAU-5′ | | 476 |
| 3 | 47 | 5′-CCGCAGCCUAUAACAACUUUCGGAA-3′ | 48.0 | 477 |
| | | 3′-GGCGUCGGAUAUUGUUGAAAGCCUU-5′ | | 478 |
| 4 | 241 | 5′-CAAGUGCUGGAGAACAUCAUGGAAA-3′ | 44.0 | 479 |
| | | 3′-GUUCACGACCUCUUGUAGUACCUUU-5′ | | 480 |
| 5 | 274 | 5′-CAGUGGCUAAUGAAGCUUGAGAAUU-3′ | 40.0 | 515 |
| | | 3′-GUCACCGAUUACUUCGAACUCUUAA-5′ | | 516 |
| 6 | 306 | 5′-GGACAACAUGAAGAAAGAAAUGGUA-3′ | 36.0 | 481 |
| | | 3′-CCUGUUGUACUUCUUUCUUUACCAU-5′ | | 482 |
| 7 | 357 | 5′-CCAGACGGCUGUGAUGAUAGAAAUA-3′ | 44.0 | 521 |
| | | 3′-GGUCUGCCGACACUACUAUCUUUAU-5′ | | 522 |
| 8 | 390 | 5′-CCUGUUGAACCAAACAGCUGAGCAA-3′ | 48.0 | 483 |
| | | 3′-GGACAACUUGGUUUGUCGACUCGUU-5′ | | 484 |
| 9 | 403 | 5′-ACAGCUGAGCAAACGCGGAAGUUAA-3′ | 48.0 | 523 |
| | | 3′-UGUCGACUCGUUUGCGCCUUCAAUU-5′ | | 524 |
| 10 | 414 | 5′-AACGCGGAAGUUAACUGAUGUGGAA-3′ | 44.0 | 525 |
| | | 3′-UUGCGCCUUCAAUUGACUACACCUU-5′ | | 526 |
| 11 | 420 | 5′-GAAGUUAACUGAUGUGGAAGCCCAA-3′ | 44.0 | 529 |
| | | 3′-CUUCAAUUGACUACACCUUCGGGUU-5′ | | 530 |
| 12 | 425 | 5′-UAACUGAUGUGGAAGCCCAAGUAUU-3′ | 40.0 | 485 |
| | | 3′-AUUGACUACACCUUCGGGUUCAUAA-5′ | | 486 |
| 13 | 427 | 5′-ACUGAUGUGGAAGCCCAAGUAUUAA-3′ | 40.0 | 531 |
| | | 3′-UGACUACACCUUCGGGUUCAUAAUU-5′ | | 532 |
| 14 | 458 | 5′-CCACGAGACUUGAACUUCAGCUCUU-3′ | 48.0 | 487 |
| | | 3′-GGUGCUCUGAACUUGAAGUCGAGAA-5′ | | 488 |
| 15 | 483 | 5′-GGAACACUCCCUCUCGACAAACAAA-3′ | 48.0 | 535 |
| | | 3′-CCUUGUGAGGGAGAGCUGUUUGUUU-5′ | | 536 |
| 16 | 524 | 5′-UGGACCAGACCAGUGAAAUAAACAA-3′ | 40.0 | 537 |
| | | 3′-ACCUGGUCUGGUCACUUUAUUUGUU-5′ | | 538 |
| 17 | 812 | 5′-CCACUGUUGCUAAAGAAGAACAAAU-3′ | 36.0 | 455 |
| | | 3′-GGUGACAACGAUUUCUUCUUGUUUA-5′ | | 456 |
| 18 | 820 | 5′-GCUAAAGAAGAACAAAUCAGCUUCA-3′ | 36.0 | 541 |
| | | 3′-CGAUUUCUUCUUGUUUAGUCGAAGU-5′ | | 542 |
| 19 | 837 | 5′-CAGCUUCAGAGACUGUGCUGAAGUA-3′ | 48.0 | 457 |
| | | 3′-GUCGAAGUCUCUGACACGACUUCAU-5′ | | 458 |
| 20 | 871 | 5′-GGACACACCACAAAUGGCAUCUACA-3′ | 48.0 | 459 |
| | | 3′-CCUGUGUGGUGUUUACCGUAGAUGU-5′ | | 460 |
| 21 | 877 | 5′-ACCACAAAUGGCAUCUACACGUUAA-3′ | 40.0 | 489 |
| | | 3′-UGGUGUUUACCGUAGAUGUGCAAUU-5′ | | 490 |
| 22 | 888 | 5′-CAUCUACACGUUAACAUUCCCUAAU-3′ | 36.0 | 461 |
| | | 3′-GUAGAUGUGCAAUUGUAAGGGAUUA-5′ | | 462 |
| 23 | 894 | 5′-CACGUUAACAUUCCCUAAUUCUACA-3′ | 36.0 | 491 |
| | | 3′-GUGCAAUUGUAAGGGAUUAAGAUGU-5′ | | 492 |
| 24 | 922 | 5′-GAGAUCAAGGCCUACUGUGACAUGG-3′ | 52.0 | 637 |
| | | 3′-CUCUAGUUCCGGAUGACACUGUACC-5′ | h/m | 638 |
| 25 | 923 | 5′-AGAUCAAGGCCUACUGUGACAUGGA-3′ | 48.0 | 639 |
| | | 3′-UCUAGUUCCGGAUGACACUGUACCU-5′ | h/m | 640 |
| 26 | 924 | 5′-GAUCAAGGCCUACUGUGACAUGGAA-3′ | 48.0 | 547 |
| | | 3′-CUAGUUCCGGAUGACACUGUACCUU-5′ | | 548 |
| 27 | 951 | 5′-UGGAGGAGGCGGGUGGACAAUUAUU-3′ | 52.0 | 463 |
| | | 3′-ACCUCCUCCGCCCACCUGUUAAUAA-5′ | | 464 |
| 28 | 962 | 5′-GGUGGACAAUUAUUCAGCGACGUGA-3′ | 48.0 | 465 |
| | | 3′-CCACCUGUUAAUAAGUCGCUGCACU-5′ | | 466 |
| 29 | 980 | 5′-GACGUGAGGAUGGCAGCGUUGAUUU-3′ | 52.0 | 551 |
| | | 3′-CUGCACUCCUACCGUCGCAACUAAA-5′ | | 552 |
| 30 | 1032 | 5′-GGGAUUUGGUAACCCUUCAGGAGAA-3′ | 48.0 | 493 |
| | | 3′-CCCUAAACCAUUGGGAAGUCCUCUU-5′ | | 494 |
| 31 | 1066 | 5′-GGAAAUGAGUUUGUUUCGCAACUGA-3′ | 40.0 | 553 |
| | | 3′-CCUUUACUCAAACAAAGCGUUGACU-5′ | | 554 |
| 32 | 1082 | 5′-CGCAACUGACUAAUCAGCAACGCUA-3′ | 48.0 | 467 |
| | | 3′-GCGUUGACUGAUUAGUCGUUGCGAU-5′ | | 468 |
| 33 | 1140 | 5′-GAAUGAGGCUUACUCAUUGUAUGAA-3′ | 36.0 | 557 |
| | | 3′-CUUACUCCGAAUGAGUAACAUACUU-5′ | | 558 |
| 34 | 1144 | 5′-GAGGCUUACUCAUUGUAUGAACAUU-3′ | 36.0 | 559 |
| | | 3′-CUCCGAAUGAGUAACAUACUUGUAA-5′ | | 560 |
| 35 | 1242 | 5′-CAGCAUCAGCCAACCAGGAAAUGAU-3′ | 48.0 | 469 |
| | | 3′-GUCGUAGUCGGUUGGUCCUUUACUA-5′ | | 470 |
| 36 | 1277 | 5′-AGGAUGGAGACAACGACAAAUGUAU-3′ | 40.0 | 563 |
| | | 3′-UCCUACCUCUGUUGCUGUUUACAUA-5′ | | 564 |
| 37 | 1283 | 5′-GAGACAACGACAAAUGUAUUUGCAA-3′ | 36.0 | 565 |
| | | 3′-CUCUGUUGCUGUUUACAUAAACGUU-5′ | | 566 |
| 38 | 1342 | 5′-GAUGCAUGUGGUCCUUCCAACUUGA-3′ | 48.0 | 495 |
| | | 3′-CUACGUACACCAGGAAGGUUGAACU-5′ | | 496 |
| 39 | 1354 | 5′-CCUUCCAACUUGAACGGAAUGUACU-3′ | 44.0 | 471 |
| | | 3′-GGAAGGUUGAACUUGCCUUACAUGA-5′ | | 472 |
| 40 | 1359 | 5′-CAACUUGAACGGAAUGUACUAUCCA-3′ | 40.0 | 567 |
| | | 3′-GUUGAACUUGCCUUACAUGAUAGGU-5′ | | 568 |
| 41 | 1390 | 5′-CAGAACACAAAUAAGUUCAACGGCA-3′ | 40.0 | 473 |
| | | 3′-GUCUUGUGUUUAUUCAAGUUGCCGU-5′ | | 474 |
| 42 | 1410 | 5′-CGGCAUUAAAUGGUACUACUGGAAA-3′ | 40.0 | 497 |
| | | 3′-GCCGUAAUUUACCAUGAUGACCUUU-5′ | | 498 |
| 43 | 1421 | 5′-GGUACUACUGGAAAGGCUCAGGCUA-3′ | 52.0 | 571 |
| | | 3′-CCAUGAUGACCUUUCCGAGUCCGAU-5′ | | 572 |
| 44 | 1447 | 5′-UCGCUCAAGGCCACAACCAUGAUGA-3′ | 52.0 | 641 |
| | | 3′-AGCGAGUUCCGGUGUUGGUACUACU-5′ | h/m | 642 |
| 45 | 1448 | 5′-CGCUCAAGGCCACAACCAUGAUGAU-3′ | 52.0 | 643 |
| | | 3′-GCGAGUUCCGGUGUUGGUACUACUA-5′ | h/m | 644 |
| 46 | 1449 | 5′-GCUCAAGGCCACAACCAUGAUGAUC-3′ | 52.0 | 645 |
| | | 3′-CGAGUUCCGGUGUUGGUACUACUAG-5′ | h/m | 646 |
| 47 | 1450 | 5′-CUCAAGGCCACAACCAUGAUGAUCC-3′ | 52.0 | 647 |
| | | 3′-GAGUUCCGGUGUUGGUACUACUAGG-5′ | h/m | 648 |
| 48 | 1623 | 5′-CAUGCUCCAGAUUAGAGCCUGUAAA-3′ | 44.0 | 605 |
| | | 3′-GUACGAGGUCUAAUCUCGGACAUUU-5′ | | 606 |

### Example 2

### High-Through-Put Screening of Human Ang-2 siRNA for Their Potency in Inhibiting Ang-2 Expression in HUVEC Cells

A reverse transfection based high-through-put (HTP) method was used to screen 48 human Ang-2 siRNAs (Table 11) for their potency in inhibiting Ang-2 expression in HUVEC cells. Briefly, 10 nM of siRNA duplex was spotted onto the bottom of a 96-well plate followed by addition of 0.25 μl of Lipofectamine™ RNAiMAX (Invitrogen). A luciferase specific 25-mer siRNA was used as the negative control. The plate was incubated at room temperature for 10-20 minutes, and 7,500 HUVEC cells in 100 ul growth medium was added to each wells. The plate was mixed gently by rocking the plate back and forth, and then incubated for 24-48 hours at 37° C. in a CO₂ incubator. The effect of siRNA mediated Ang-2 knockdown was monitored by measuring the concentration of Ang-2 protein in the medium using a human Ang-2 ELISA kit (R&D). The cell viability of the transfected cells was measured using a WST-1 assay kit (Roche) for normalization of Ang-2 concentration.

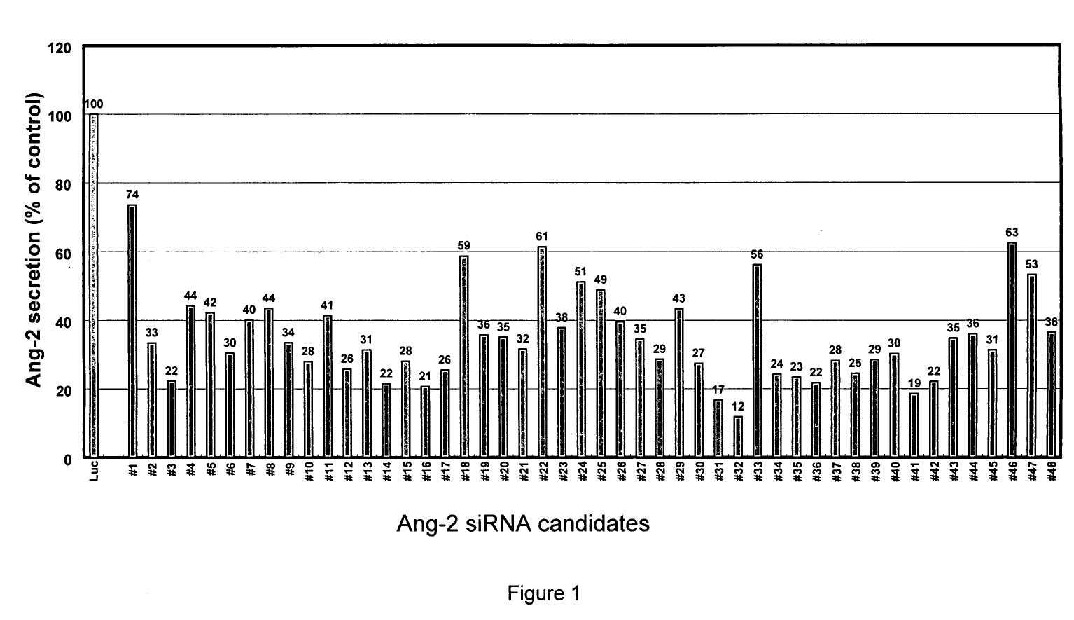

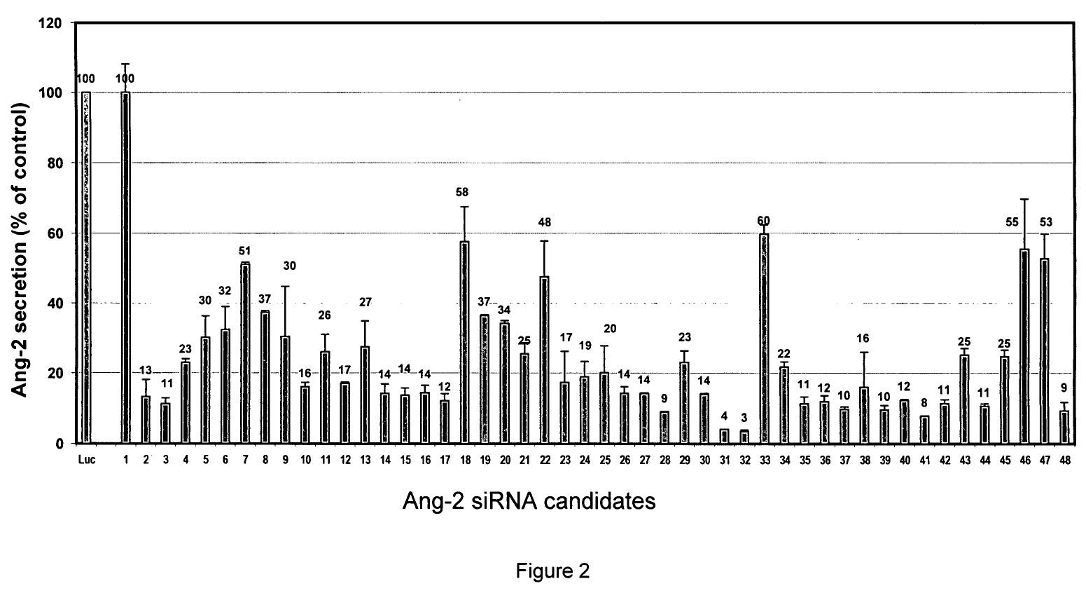

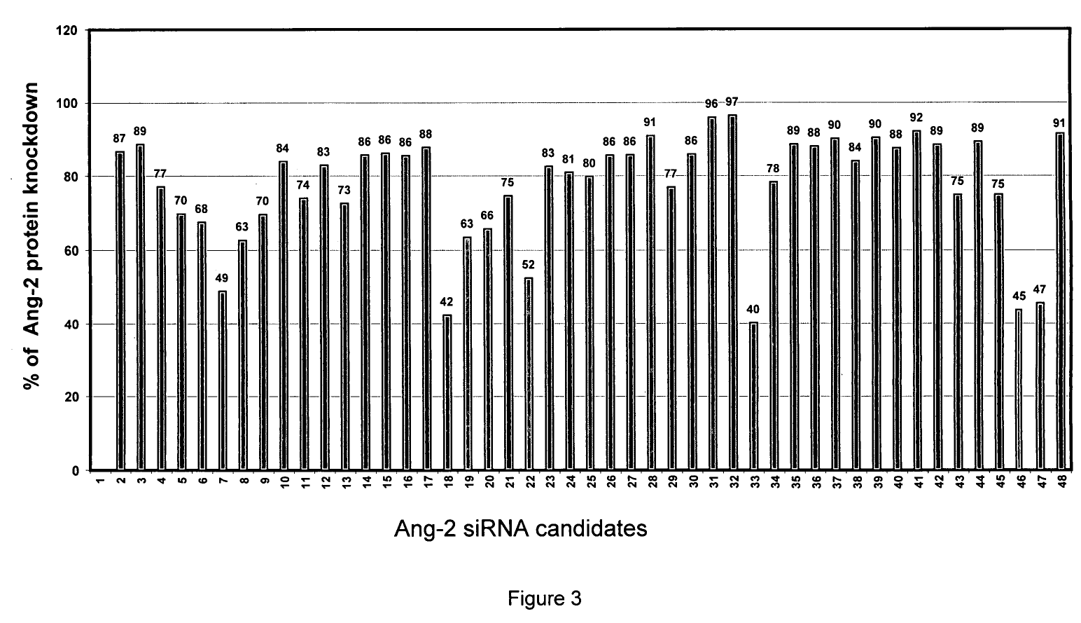

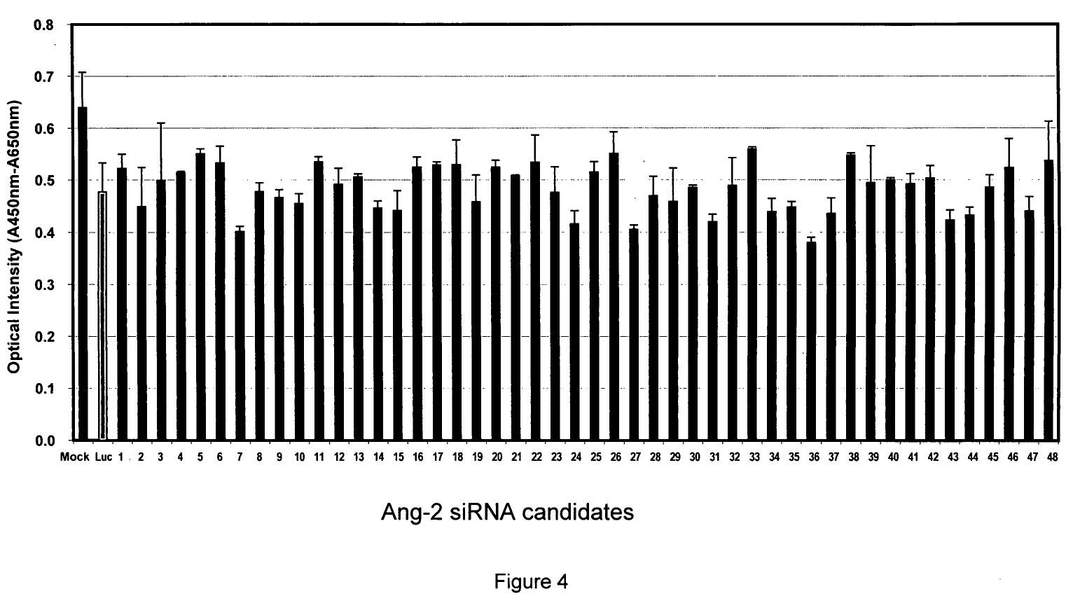

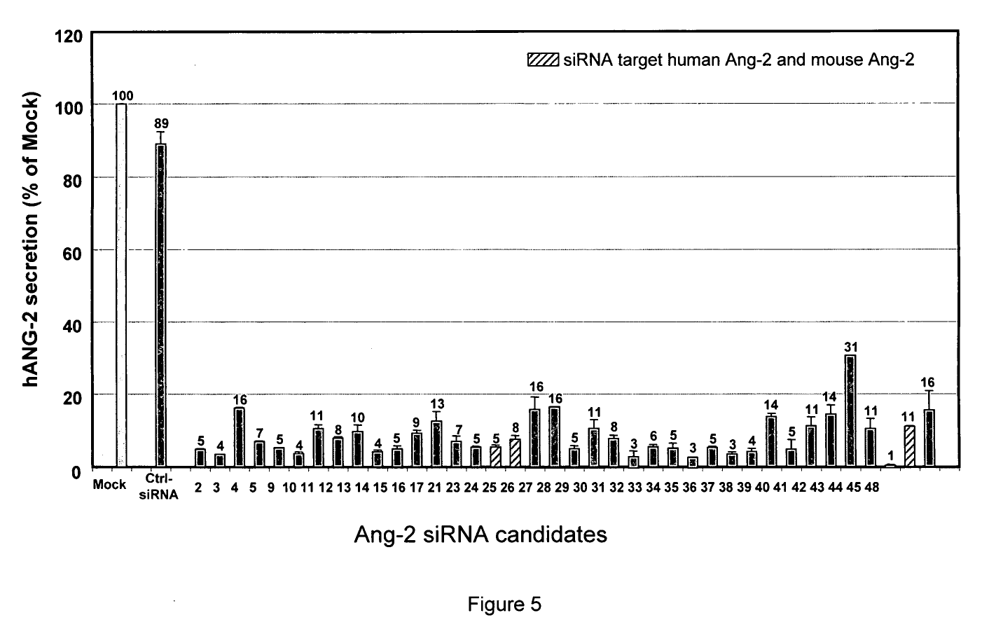

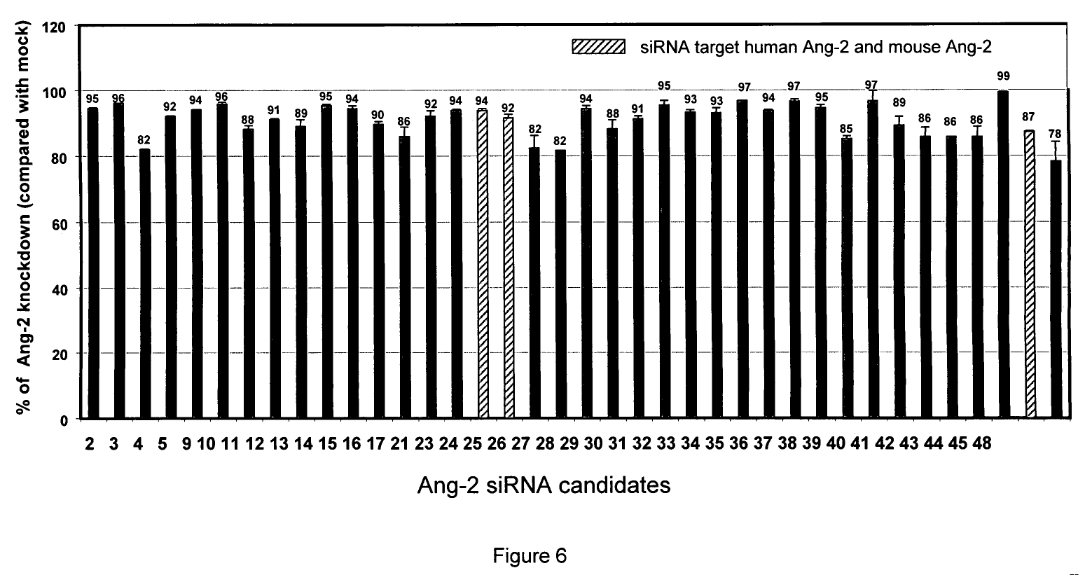

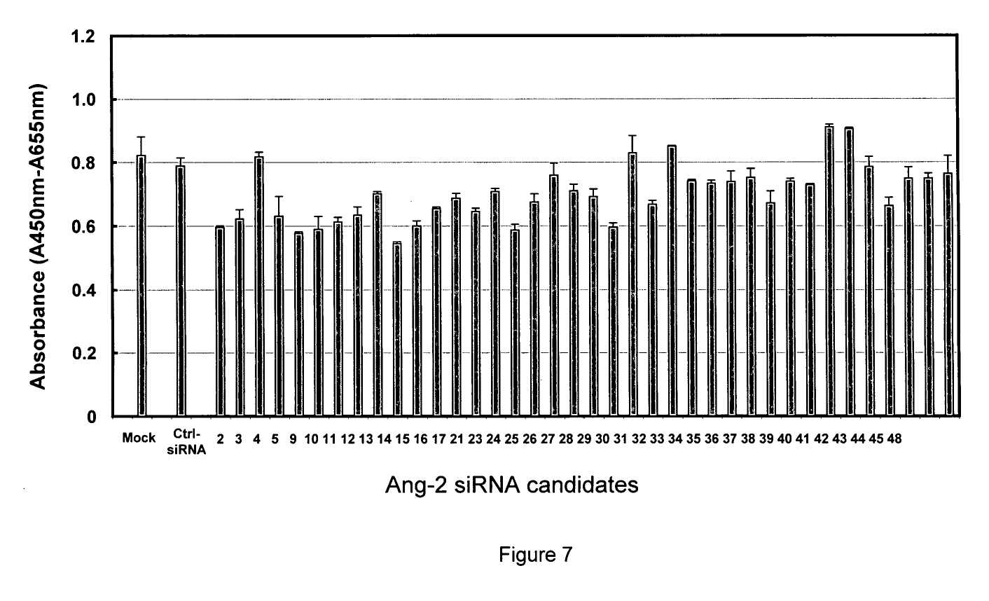

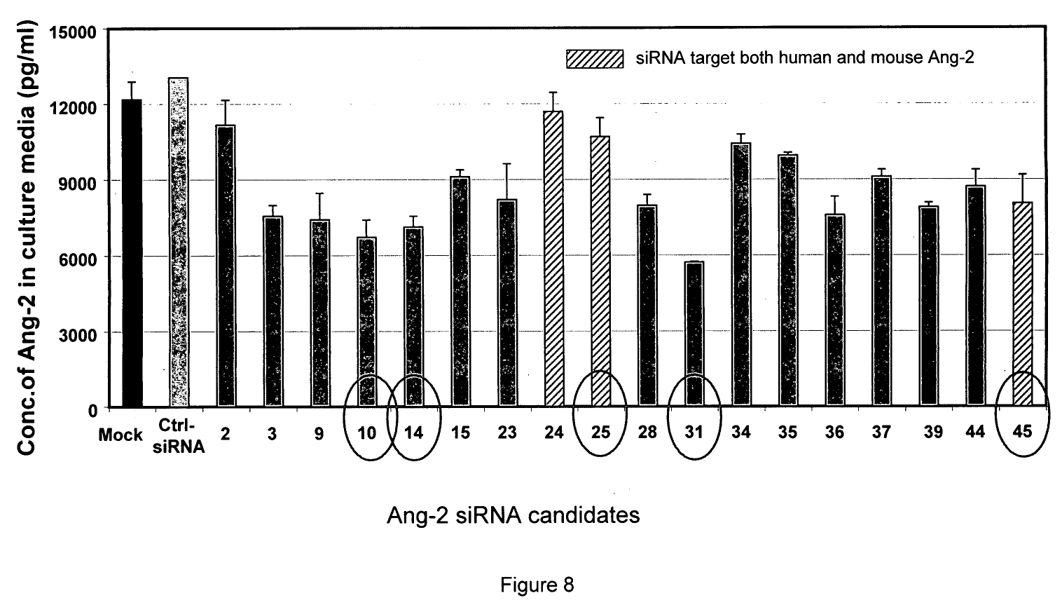

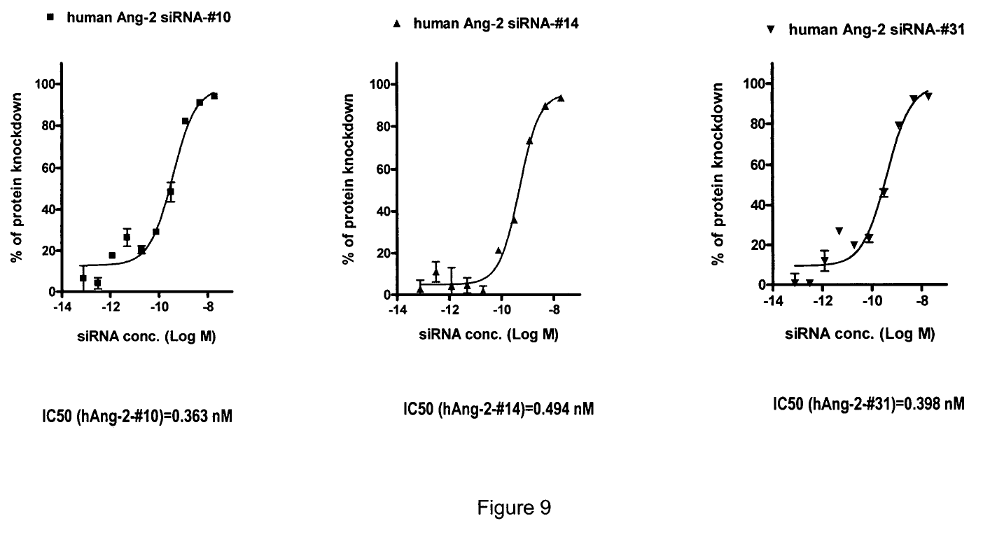

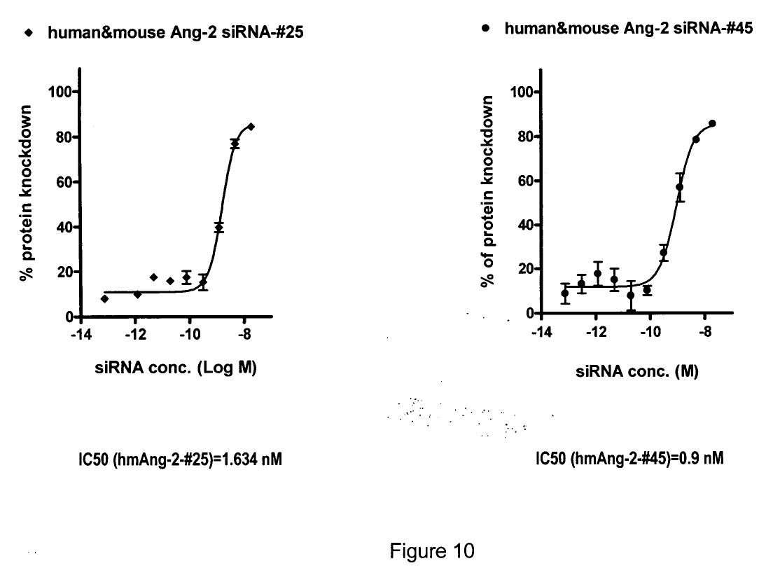

Significant inhibition of Ang-2 protein level expression in transfected HUVEC cells was observed at 24 hours post transfection with a majority of the 48 Ang-2 siRNA candidates tested (FIG. 1). At 48 hours post transfection, the inhibition effects were more profound (FIG. 2), with about 50% of the Ang-2 siRNA candidates showing a greater than 80% inhibition of Ang-2 expression compared to cells transfected with control Luc-siRNA (FIG. 3). There was no cytotoxicity in the transfected HUVEC cells that associated with knockdown of Ang-2 expression (FIG. 4).

### Example 3

### Confirmation of Ang-2 Gene Expression Knockdown in HUVEC Cells Transfected with 2 nM Ang-2 siRNA

In a separate experiment, 38 Ang-2 siRNA candidates that demonstrated a high percentage of Ang-2 knockdown in previous HTP screening (FIG. 1-3) were further examined for their potency in inhibiting Ang-2 expression in HUVEC cells using a reverse transfection method. Briefly, 2 nM of siRNA duplex was spotted onto the bottom of a 96-well plate followed by addition of 0.25 μl of Lipofectamine™ RNAiMAX (Invitrogen). A negative control (Ctrl-) siRNA, which has a 19-nt double-stranded region with dTdT 3′-overhangs on both strands and does not has a significant homologous sequence with any known human gene, was used as the negative control. The plate was incubated at room temperature for 10-20 minutes, and 7,500 HUVEC cells in 100 μl growth medium was added to each well. The plate was mixed gently by rocking the plate back and forth, and then incubated for 48 hours at 37° C. in a CO₂ incubator. The effect of siRNA mediated Ang-2 knockdown was monitored by measuring the concentration of Ang-2 protein in the medium using a human Ang-2 ELISA kit (R&D). The cell viability of the transfected cells was measured using a WST-1 assay kit (Roche) for normalization of Ang-2 concentration.

Significant inhibition (>90%) of Ang-2 protein level expression in transfected HUVEC cells was observed at 48 hours post transfection with a majority of the 38 Ang-2 siRNA candidates tested (FIG. 5), including many siRNA candidates with a greater than 90% knockdown of Ang-2 protein level expression (FIG. 6). In addition, 3 siRNA that target both human and mouse Ang-2 also demonstrated high potency in knocking down human Ang-2 expression (FIGS. 5 and 6). Finally, there was no cytotoxicity in the transfected HUVEC cells that associated with knockdown of Ang-2 expression (FIG. 7).

### Example 4

### Final Selection of Ang-2 siRNA Based on Ang-2 Gene Expression Knockdown in HUVEC Cells Transfected with 0.2 nM

In another experiment, 18 Ang-2 siRNA candidates that demonstrated a higher than 94% knockdown of Ang-2 expression in a previous experiment (FIG. 6) and 3 human/mouse Ang-2 siRNA were further examined for their potency in inhibiting Ang-2 expression in HUVEC cells using a reverse transfection method with a lower dose of siRNA. Briefly, 0.2 nM of siRNA duplex was spotted onto the bottom of a 96-well plate followed by addition of 0.25 μl of Lipofectamine™ RNAiMAX (Invitrogen). A negative control (Ctrl-) siRNA, which has a 19-nt double-stranded region with dTdT 3′-overhangs on both strands and does not has a significant homologous sequence with any known human gene, was used as the negative control. The plate was incubated at room temperature for 10-20 minutes, and 7,500 HUVEC cells in 100 μl growth medium was added to each well. The plate was mixed gently by rocking the plate back and forth, and then incubated for 48 hours at 37° C. in a CO₂ incubator. The effect of siRNA mediated Ang-2 knockdown was monitored by measuring the concentration of Ang-2 protein in the medium using a human Ang-2 ELISA kit (R&D). The cell viability of the transfected cells was measured using a WST-1 assay kit (Roche) for normalization of Ang-2 concentration.

When transfected with only 0.2 nM of siRNA, significant inhibition (30-50%) of Ang-2 protein level expression in transfected HUVEC cells was observed at 48 hours post transfection with a majority of the 38 Ang-2 siRNA candidates tested (FIG. 8), including one siRNA which targets both human and mouse Ang-2.

Three Ang-2 siRNA, #10 (Ang-2-25-10), #14 (Ang-2-25-14), and #31 (Ang-2-25-31) were selected for further experiments as Ang-2 siRNA. In addition, #25 (Ang-2-25-25) and #45 (Ang-2-25-45) were selected for further experiments as human/mouse Ang-2 siRNA.

### Example 5

### Determination of IC50 Values of Ang-2 siRNA

Upon the confirmation of Ang-2 siRNA candidates, experiments were conducted to determine the IC50 value of Ang-2 siRNA (Ang-2-25-10, Ang-2-25-14, and Ang-2-25-31) in HUVEC cells. Briefly, 10 dilutions of each siRNA duplex were spotted onto the bottom of a 96-well plate followed by addition of 0.25 μl of Lipofectamine™ RNAiMAX (Invitrogen). The siRNA dilutions were 0.076 pM, 0.31 pM, 1.2 pM, 4.9 pM, 19.5 pM, 78.1 pM, 312.5 pM, 1.25 nM, 5 nM, and 20 nM. The plate was incubated at room temperature for 10-20 minutes, and 7,500 HUVEC cells in 100 μl growth medium was added to each well. The plate was mixed gently by rocking the plate back and forth, and then incubated for 48 hours at 37° C. in a CO₂ incubator. The effect of siRNA-mediated Ang-2 knockdown was monitored by measuring the concentration of Ang-2 protein in the medium using a human Ang-2 ELISA kit (R&D). The cell viability of the transfected cells was measured using a WST-1 assay kit (Roche) for normalization of Ang-2 concentration.

The IC50 value of each siRNA duplex in HUVEC cells at 48 hours post siRNA transfection was obtained using the GraphPad Prism program (FIG. 9). The IC50 of Ang-2-25-10 was 0.363 nM, the IC50 of Ang-2-25-14 was 0.494 nM, and the IC50 of Ang-2-25-31 was 0.398 nM (FIG. 9 and Table 12).

### Example 6

### Determination of IC50 Values of Human/Mouse Ang-2 siRNA

Upon the confirmation of human/mouse Ang-2 siRNA candidates that target both human and mouse Ang-2 mRNA, experiments were conducted to determine the IC50 value of human/mouse Ang-2 siRNA (Ang-2-25-25 and Ang-2-25-45) in HUVEC cells. Briefly, 10 dilutions of each siRNA duplex were spotted onto the bottom of a 96-well plate followed by addition of 0.25 μl of Lipofectamine™ RNAiMAX (Invitrogen). The siRNA dilutions were 0.076 pM, 0.31 pM, 1.2 pM, 4.9 pM, 19.5 pM, 78.1 pM, 312.5 pM, 1.25 nM, 5 nM, and 20 nM. The plate was incubated at room temperature for 10-20 minutes, and 7,500 HUVEC cells in 100 μl growth medium was added to each well. The plate was mixed gently by rocking the plate back and forth, and then incubated for 48 hours at 37° C. in a CO₂ incubator. The effect of siRNA-mediated Ang-2 knockdown was monitored by measuring the concentration of Ang-2 protein in the medium using a human Ang-2 ELISA kit (R&D). The cell viability of the transfected cells was measured using a WST-1 assay kit (Roche) for normalization of Ang-2 concentration.

The IC50 value of each siRNA duplex in HUVEC cells at 48 hours post siRNA transfection was obtained using the GraphPad Prism program (FIG. 10). The IC50 of Ang-2-25-25 was 1.634 nM, and the IC50 of Ang-2-25-45 was 0.90 nM (FIG. 10 and Table 12).

TABLE 12
IC50 of selected Ang-2-siRNA in transfected HUVEC cells

| | IC50 (nM) |
|---|---|
| siRNA | 48 hours post-transfection |
| human Ang-2-25mer-siRNA#10 | 0.363 |
| human Ang-2-25mer-siRNA#14 | 0.494 |
| human Ang-2-25mer-siRNA#31 | 0.398 |
| human&mouse Ang-2-25mer-siRNA#25 | 1.634 |
| human&mouse Ang-2-25mer-siRNA#45 | 0.9 |

TABLE 13
Ang-1, Ang-2, and Tie2 mRNA sequence table
Gene: TEK (Tie2)
Species: human
NCBI Accession No.: NM_000459
SEQ ID NO: 649
Sequence:
AGTTTCCCGCCTATGAGAGGATACCCCTATTGTTTCTGAAAATGCTGAC
CGGGACCCACACTTCCAACAAAAATTCCTCTGCCCCTACAGCAGCAGC
AAAAGCAGCAGCAGAAGCAACAGCAACAGATAAGTGTTTTGATGAATT
GCGAGATGGATAGGGCTTGAGTGCCCCCAGCCCTGCTGATACCAAATG
CCTTTAAGATACAGCCTTTCCCATCCTAATCTACAAAGGAAACAGGAA
AAAGGAACTTAAAACTCCCTGTGCTCAGACAGAAATGAGACTGTTACA
GCCTGCTTCTGTGCTGTTCCTTCTTGCCTCTAACTTGTAAACAAGACGT
AGTAGGACGATGCTAATGGAAAGTCACAAACCGCTGGGTTTTTGAAAGG
ATCCTTGGGACCTCATGCACATTTGTGGAAACTGGATGGAGAGATTTGG
GGAAGCATGGACTCTTTAGCCAGCTTAGTTCTCTGTGGAGTCAGCTTGC
TCCTTTCTGGAACTGTGGAAGGTGCCATGGACTTGATCTTGATCAATTC
CCTACCTCTTGTATCTGATGCTGAAACATCTCTCACCTGCATTGCCTCT
GGGTGGCGCCCCCATGAGCCCATCACCATAGGAAGGGACTTTGAAGCCT
TAATGAACCAGCACCAGGATCCGCTGGAAGTTACTCAAGATGTGACCA
GAGAATGGGCTAAAAAAGTTGTTTGGAAGAGAGAAAAGGCTAGTAAG
ATCAATGGTGCTTATTTCTGTGAAGGGCGAGTTCGAGGAGAGGCAATC
AGGATACGAACCATGAAGATGCGTCAACAAGCTTCCTTCCTACCAGCT
ACTTTAACTATGACTGTGGACAAGGGAGATAACGTGAACATATCTTTCA
AAAAGGTATTGATTAAAGAAGAAGATGCAGTGATTTACAAAAATGGTT
CCTTCATCCATTCAGTGCCCCGGCATGAAGTACCTGATATTCTAGAAGT
ACACCTGCCTCATGCTCAGCCCCAGGATGCTGGAGTGTACTCGGCCAG
GTATATAGGAGGAAACCTCTTCACCTCGGCCTTCACCAGGCTGATAGTC
CGGAGATGTGAAGCCCAGAAGTGGGGACCTGAATGCAACCATCTCTGT
ACTGCTTGTATGAACAATGGTGTCTGCCATGAAGATACTGGAGAATGC
ATTTGCCCTCCTGGGTTTATGGGAAGGACGTGTGAGAAGGCTTGTGAAC
TGCACACGTTTGGCAGAACTTGTAAAGAAAGGTGCAGTGGACAAGAGG
GATGCAAGTCTTATGTGTTCTGTCTCCCTGACCCCTATGGGTGTTCCTG
TGCCACAGGCTGGAAGGGTCTGCAGTGCAATGAAGCATGCCACCCTGGT
TTTTACGGGCCAGATTGTAAGCTTAGGTGCAGCTGCAACAATGGGGAG
ATGTGTGATCGCTTCCAAGGATGTCTCTGCTCTCCAGGATGGCAGGGGC
TCCAGTGTGAGAGAGAAGGCATACCGAGGATGACCCCAAAGATAGTGG
ATTTGCCAGATCATATAGAAGTAAACAGTGGTAAATTTAATCCCATTTG
CAAAGCTTCTGGCTGGCCGCTACCTACTAATGAAGAAATGACCCTGGT
GAAGCCGGATGGGACAGTGCTCCATCCAAAAGACTTTAACCATACGGA
TCATTTCTCAGTAGCCATATTCACCATCCACCGGATCCTCCCCCCTGAC
TCAGGAGTTTGGGTCTGCAGTGTGAACACAGTGGCTGGGATGGTGGAAA
AGCCCTTCAACATTTCTGTTAAAGTTCTTCCAAAGCCCCTGAATGCCCC
AAACGTGATTGACACTGGACATAACTTTGCTGTCATCAACATCAGCTCT
GAGCCTTACTTTGGGGATGGACCAATCAAATCCAAGAAGCTTCTATAC
AAACCCGTTAATCACTATGAGGCTTGGCAACATATTCAAGTGACAAAT
GAGATTGTTACACTCAACTATTTGGAACCTCGGACAGAATATGAACTCT
GTGTGCAACTGGTCCGTCGTGGAGAGGGTGGGGAAGGGCATCCTGGAC
CTGTGAGACGCTTCACAACAGCTTCTATCGGACTCCCTCCTCCAAGAGG
TCTAAATCTCCTGCCTAAAAGTCAGACCACTCTAAATTTGACCTGGCAA
CCAATATTTCCAAGCTCGGAAGATGACTTTTATGTTGAAGTGGAGAGA
AGGTCTGTGCAAAAAAGTGATCAGCAGAATATTAAAGTTCCAGGCAAC
TTGACTTCGGTGCTACTTAACAACTTACATCCCAGGGAGCAGTACGTGG
TCCGAGCTAGAGTCAACACCAAGGCCCAGGGGGAATGGAGTGAAGATC
TCACTGCTTGGACCCTTAGTGACATTCTTCCTCCTCAACCAGAAAACAT
CAAGATTTCCAACATTACACACTCCTCAGCTGTGATTTCTTGGACAATA
TTGGATGGCTATTCTATTTCTTCTATTACTATCCGTTACAAGGTTCAAG
GCAAGAATGAAGACCAGCACGTTGATGTGAAGATAAAGAATGCCACCAT
CACTCAGTATCAGCTCAAGGGCCTAGAGCCTGAAACAGCATACCAGGT
GGACATTTTTGCAGAGAACAACATAGGGTCAAGCAACCCAGCCTTTTCT
CATGAACTGGTGACCCTCCCAGAATCTCAAGCACCAGCGGACCTCGGA
GGGGGGAAGATGCTGCTTATAGCCATCCTTGGCTCTGCTGGAATGACCT
GCCTGACTGTGCTGTTGGCCTTTCTGATCATATTGCAATTGAAGAGGGC
AAATGTGCAAAGGAGAATGGCCCAAGCCTTCCAAAACGTGAGGGAAG
AACCAGCTGTGCAGTTCAACTCAGGGACTCTGGCCCTAAACAGGAAGG
TCAAAAACAACCCAGATCCTACAATTTATCCAGTGCTTGACTGGAATGA
CATCAAATTTCAAGATGTGATTGGGGAGGGCAATTTTGGCCAAGTTCTT
AAGGCGCGCATCAAGAAGGATGGGTTACGGATGGATGCTGCCATCAAA
AGAATGAAAGAATATGCCTCCAAAGATGATCACAGGGACTTTGCAGGA
GAACTGGAAGTTCTTTGTAAACTTGGACACCATCCAAACATCATCAATC
TCTTAGGAGCATGTGAACATCGAGGCTACTTGTACCTGGCCATTGAGTA
CGCGCCCCATGGAAACCTTCTGGACTTCCTTCGCAAGAGCCGTGTGCTG
GAGACGGACCCAGCATTTGCCATTGCCAATAGCACCGCGTCCACACTG
TCCTCCCAGCAGCTCCTTCACTTCGCTGCCGACGTGGCCCGGGGCATGG
ACTACTTGAGCCAAAAACAGTTTATCCACAGGGATCTGGCTGCCAGAA
ACATTTTAGTTGGTGAAAACTATGTGGCAAAAATAGCAGATTTTGGATT
GTCCCGAGGTCAAGAGGTGTATGTGAAAAAGACAATGGGAAGGCTCCC
AGTGCGCTGGATGGCCATCGAGTCACTGAATTACAGTGTGTACACAAC
CAACAGTGATGTATGGTCCTATGGTGTGTTACTATGGGAGATTGTTAGC
TTAGGAGGCACACCCTACTGCGGGATGACTTGTGCAGAACTCTACGAG
AAGCTGCCCCAGGGCTACAGACTGGAGAAGCCCCTGAACTGTGATGAT
GAGGTGTATGATCTAATGAGACAATGCTGGCGGGAGAAGCCTTATGAG
AGGCCATCATTTGCCCAGATATTGGTGTCCTTAAACAGAATGTTAGAGG
AGCGAAAGACCTACGTGAATACCACGCTTTATGAGAAGTTTACTTATGC
AGGAATTGACTGTTCTGCTGAAGAAGCGGCCTAGGACAGAACATCTGT
ATACCCTCTGTTTCCCTTTCACTGGCATGGGAGACCCTTGACACCTGCT
GAGAAAACATGCCTCTGCCAAAGGATGTGATATATAAGTGTACATATG
TGCTGTACACCTGGGACCTTCACCACTGTAGATCCCATGCATGGATCTA
TGTAGTATGCTCTGACTCTAATAGGACTGTATATACTGTTTTAAGAATG
GGCTGAAATCAGAATGCCTGTTTGTGGTTTCATATGCAATAATATATTT
TTTTAAAAATGTGGACTTCATAGGAAGGC GTGAGTACAATTAGTATAA
TGCATAACTCATTGTTGTCCTAGATATTTTGATATTTACCTTTATGTTG
AATGCTATTAAATGTTTTCCTGTGTCAAAGTAAAATATTGTTAATAAAC
CTAACAATGACCCTGATAGTACAGGTTAAGTGAGAGAACTATATGAATT
CTAACAAGTCATAGGTTAATATTTAAGACACTGAAAAATCTAAGTGATA
TAAATCAGATTCTTCTCTCTCAATTTTATCCCTCACCTGTAGCAGCCAG
TCCCGTTTCATTTAGTCATGTGACCACTCTGTCTTGTGTTTCCACAGCC
TGCAAGTCAGTCCAGGATGCTAACATCTAAAAATAGACTTAAATCTCAT
TGCTTACAAGCCTAAGAATCTTTAGAGAAGTATACATAAGTTTAGGATA
AAATAATGGGATTTTCTTTTCTTTTCTCTGGTAATATTGACTTGTATAT
TTTAAGAAATAACAGAAAGCCTGGGTGACATTTGGGAGACATGTGACAT
TTATATATTGAATTAATATCCCTACATGTATTGCACATTGTAAAAAGTT
TTAGTTTTGATGAGTTGTGAGTTTACCTTGTATACTGTAGGCACACTTT
GCACTGATATATCATGAGTGAATAAATGTCTTGCCTACTCACGTCTCAA
AAAAAAAAAAAAAAAAAAAAAAAAAAAAAAAAAAAAAAAAAAAAAAAA
AAAAAAAAA
Gene: TEK (Tie2)
Species: mouse
NCBI Accession No.: NM_013690
SEQ ID NO: 650
Sequence:
GAGCAGGAGCCGGAGCAGGAGCAGAAGATAAGCCTTGGATGAAGGGC
AAGATGGATAGGGCTCGCTCTGCCCCAAGCCCTGCTGATACCAAGTGC
CTTTAAGATACAGCCTTTCCCATCCTAATCTGCAAAGGAAACAGGAAA
AAGGAACTTAACCCTCCCTGTGCTCAGACAGAAATGAGACTGTTACCG
CCTGCTTCTGTGGTGTTTCTCCTTGCCGCCAACTTGTAAACAAGAGCGA
GTGGACCATGCGAGCGGGAAGTCGCAAAGTTGTGAGTTGTTGAAAGCT
TCCCAGGGACTCATGCTCATCTGTGGACGCTGGATGGGGAGATCTGGG
GAAGTATGGACTCTTTAGCCGGCTTAGTTCTCTGTGGAGTCAGCTTGCT
CCTTTATGGAGTAGTAGAAGGCGCCATGGACCTGATCTTGATCAATTCC
CTACCTCTTGTGTCTGATGCCGAAACATCCCTCACCTGCATTGCCTCTG
GGTGGCACCCCCATGAGCCCATCACCATAGGAAGGGACTTTGAAGCCT
TAATGAACCAGCACCAAGATCCACTGGAGGTTACTCAAGATGTGACCA
GAGAATGGGCGAAAAAAGTTGTTTGGAAGAGAGAAAAGGCCAGTAAG
ATTAATGGTGCTTATTTCTGTGAAGGTCGAGTTCGAGGACAGGCTATAA
GGATACGGACCATGAAGATGCGTCAACAAGCATCCTTCCTACCTGCTA
CTTTAACTATGACCGTGGACAGGGGAGATAATGTGAACATATCTTTCAA
AAAGGTGTTAATTAAAGAAGAAGATGCAGTGATTTACAAAAATGGCTC
CTTCATCCACTCAGTGCCCCGGCATGAAGTACCTGATATTTTAGAAGTT
CACTTGCCGCATGCTCAGCCCCAGGATGCTGGTGTGTACTCGGCCAGGT
ACATAGGAGGAAACCTGTTCACCTCAGCCTTCACCAGGCTGATTGTTCG
GAGATGTGAAGCTCAGAAGTGGGGGCCCGACTGTAGCCGTCCTTGTAC
TACTTGCAAGAACAATGGAGTCTGCCATGAAGATACCGGGGAATGCAT
TTGCCCTCCTGGGTTTATGGGGAGAACATGTGAGAAAGCTTGTGAGCC
GCACACATTTGGCAGGACCTGTAAAGAAAGGTGTAGTGGACCAGAAGG
ATGCAAGTCTTATGTGTTCTGTCTCCCAGACCCTTACGGGTGTTCCTGT
GCCACAGGCTGGAGGGGGTTGCAGTGCAATGAAGCATGCCCATCTGGTT
ACTACGGACCAGACTGTAAGCTCAGGTGCCACTGTACCAATGAAGAGA
TATGTGATCGGTTCCAAGGATGCCTCTGCTCTCAAGGATGGCAAGGGCT
GCAGTGTGAGAAAGAAGGCAGGCCAAGGATGACTCCACAGATAGAGG
ATTTGCCAGATCACATTGAAGTAAACAGTGGAAAATTTAACCCCATCTG
CAAAGCCTCTGGGTGGCCACTACCTACTAGTGAAGAAATGACCCTAGT
GAAGCCAGATGGGACAGTGCTCCAACCAAATGACTTCAACTATACAGA
TCGTTTCTCAGTGGCCATATTCACTGTCAACCGAGTCTTACCTCCTGAC
TCAGGAGTCTGGGTCTGCAGTGTGAACACAGTGGCTGGGATGGTGGAAA
AGCCTTTCAACATTTCCGTCAAAGTTCTTCCAGAGCCCCTGCACGCCCC
AAATGTGATTGACACTGGACATAACTTTGCTATCATCAATATCAGCTCT
GAGCCTTACTTTGGGGATGGACCCATCAAATCCAAGAAGCTTTTCTATA
AACCTGTCAATCAGGCCTGGAAATACATTGAAGTGACGAATGAGATTT
TCACTCTCAACTACTTGGAGCCGCGGACTGACTACGAGCTGTGTGTGCA
GCTGGCCCGTCCTGGAGAGGGTGGAGAAGGGCATCCTGGGCCTGTGAG
ACGATTTACAACAGCGTCTATCGGACTCCCTCCTCCAAGAGGTCTCAGT
CTCCTGCCAAAAAGCCAGACAGCTCTAAATTTGACTTGGCAACCGATAT
TTACAAACTCAGAAGATGAATTTTATGTGGAAGTCGAGAGGCGATCCC
TGCAAACAACAAGTGATCAGCAGAACATCAAAGTGCCTGGGAACCTGA
CCTCGGTGCTACTGAGCAACTTAGTCCCCAGGGAGCAGTACACAGTCC
GAGCTAGAGTCAACACCAAGGCGCAGGGGGAGTGGAGTGAAGAACTC
AGGGCCTGGACCCTTAGTGACATTCTCCCTCCTCAACCAGAAAACATCA
AGATCTCCAACATCACTGACTCCACAGCTATGGTTTCTTGGACAATAGT
GGATGGCTATTCGATTTCTTCCATCATCATCCGGTATAAGGTTCAGGGC
AAAAATGAAGACCAGCACATTGATGTGAAGATCAAGAATGCTACCGTT
ACTCAGTACCAGCTCAAGGGCCTAGAGCCAGAGACTACATACCATGTG
GATATTTTTGCTGAGAACAACATAGGATCAAGCAACCCAGCCTTTTCTC
ATGAACTGAGGACGCTTCCACATTCCCCAGCCTCTGCAGACCTCGGAG
GGGGAAAGATGCTACTCATAGCCATCCTTGGGTCGGCTGGAATGACTT
GCATCACCGTGCTGTTGGCGTTTCTGATTATGTTGCAACTGAAGAGAGC
AAATGTCCAAAGGAGAATGGCTCAGGCATTCCAGAACGTGAGAGAAG
AACCAGCTGTGCAGTTTAACTCAGGAACTCTGGCCCTTAACAGGAAGG
CCAAAAACAATCCGGATCCCACAATTTATCCTGTGCTTGACTGGAATGA
CATCAAGTTTCAAGACGTGATCGGAGAGGGCAACTTTGGCCAGGTTCT
GAAGGCACGCATCAAGAAGGATGGGTTACGGATGGATGCCGCCATCAA
GAGGATGAAAGAGTATGCCTCCAAAGATGATCACAGGGACTTCGCAGG
AGAACTGGAGGTTCTTTGTAAACTTGGACACCATCCAAACATCATTAAT
CTCTTGGGAGCATGTGAACACCGAGGCTATTTGTACCTAGCTATTGAGT
ATGCCCCGCATGGAAACCTCCTGGACTTCCTGCGTAAGAGCAGAGTGC
TAGAGACAGACCCTGCTTTTGCCATCGCCAACAGTACAGCTTCCACACT
GTCCTCCCAACAGCTTCTTCATTTTGCTGCAGATGTGGCCCGGGGGATG
GACTACTTGAGCCAGAAACAGTTTATCCACAGGGACCTGGCTGCCAGA
AACATTTTAGTTGGTGAAAACTACATAGCCAAAATAGCAGATTTTGGA
TTGTCACGAGGTCAAGAAGTGTATGTGAAAAAGACAATGGGAAGGCTC
CCAGTGCGTTGGATGGCAATCGAATCACTGAACTATAGTGTCTATACAA
CCAACAGTGATGTCTGGTCCTATGGTGTATTGCTCTGGGAGATTGTTAG
CTTAGGAGGCACCCCCTACTGCGGCATGACGTGCGCGGAGCTCTATGA
GAAGCTACCCCAGGGCTACAGGCTGGAGAAGCCCCTGAACTGTGATGA
TGAGGTGTATGATCTAATGAGACAGTGCTGGAGGGAGAAGCCTTATGA
GAGACCATCATTTGCCCAGATATTGGTGTCCTTAAACAGGATGCTGGAA
GAACGGAAGACATACGTGAACACCACACTGTATGAGAAGTTTACCTAT
GCAGGAATTGACTGCTCTGCGGAAGAAGCAGCCTAGAGCAGAACTCTT
CATGTACAACGGCCATTTCTCCTCACTGGCGCGAGAGCGCCTTGACACC
TGTACCAAGCAAGCCACCCACTGCCAAGAGATGTGATATATAAGTGTA
TATATTGTGCTGTGTTTGGGACCCTCCTCATACAGCTCGTGCGGATCTG
CAGTGTGTTCTGACTCTAATGTGACTGTATATACTGCTCGGAGTAAGAA
TGTGCTAAGATCAGAATGCCTGTTCGTGGTTTCATATAATATATTTTTC
TAAAAGCATAGATTGCACAGGAAGGTATGAGTACAAATACTGTAATGCA
TAACTTGTTATTGTCCTAGATGTGTTTGATATTTTTCCTTTACAACTGA
ATGCTATAAAAGTGTTTTGCTGTGTACACATAAGATACTGTTCGTTAAA
ATAAGCATTCCCTTGACAGCACAGGAAGAAAAGCGAGGGAAATGTATGG
ATTATATTAAATGTGGGTTACTACACAAGAGGCCGAACATTCCAAGTA
GCAGAAGAGAGGGTCTCTCAACTCTGCTCCTCACCTGCAGAAGCCAGT
TTGTTTGGCCATGTGACAATTGTCCTGTGTTTTTATAGCACCCAAATCA
TTCTAAAATATGAACATCTAAAAACTTTGCTAGGAGACTAAGAACCTTT
GGAGAGATAGATATAAGTACGGTCAAAAAACAAAACTGTGGGACTTACA
TTTATTTTCTATAGTAATCTGTTGTACATTTTAAGAAGTAAAACTAGGA
ATTTAGGAGTGATGTGTGACATTTCTGACATGGAGTTACCATCCCCACA
TGTATCACATACTGTCATATTCCCACATGTATCACACATGTATTGTAAA
ATTTTGTAGTTTTGATCACTTGTGAATTTACTGTTGATGTGGTAGCCAC
CTGCTGCAATGGTTCCTCTTGTAGGTGAATAAATGTCTTGTCTACCCAC
A
Gene: ANGPT1 (Ang-1)
Species: human
NCBI Accession No.: NM_001146
SEQ ID NO: 651
Sequence:
GGGGCACACTCATGCATTCCTGTCAAGTCATCTTGTGAAAGGCTGCCTG
CTTCCAGCTTGGCTTGGATGTGCAACCTTAATAAAACTCACTGAGGTCT
GGGAGAAAATAGCAGATCTGCAGCAGATAGGGTAGAGGAAAGGGTCT
AGAATATGTACACGCAGCTGACTCAGGCAGGCTCCATGCTGAACGGTC
ACACAGAGAGGAAACAATAAATCTCAGCTACTATGCAATAAATATCTC
AAGTTTTAACGAAGAAAAACATCATTGCAGTGAAATAAAAAATTTTAA
AATTTTAGAACAAAGCTAACAAATGGCTAGTTTTCTATGATTCTTCTTC
AAACGCTTTCTTTGAGGGGGAAAGAGTCAAACAAACAAGCAGTTTTAC
CTGAAATAAAGAACTAGTTTTAGAGGTCAGAAGAAAGGAGCAAGTTTT
GCGAGAGGCACGGAAGGAGTGTGCTGGCAGTACAATGACAGTTTTCCT
TTCCTTTGCTTTCCTCGCTGCCATTCTGACTCACATAGGGTGCAGCAAT
CAGCGCCGAAGTCCAGAAAACAGTGGGAGAAGATATAACCGGATTCAA
CATGGGCAATGTGCCTACACTTTCATTCTTCCAGAACACGATGGCAACT
GTCGTGAGAGTACGACAGACCAGTACAACACAAACGCTCTGCAGAGAG
ATGCTCCACACGTGGAACCGGATTTCTCTTCCCAGAAACTTCAACATCT
GGAACATGTGATGGAAAATTATACTCAGTGGCTGCAAAAACTTGAGAA
TTACATTGTGGAAAACATGAAGTCGGAGATGGCCCAGATACAGCAGAA
TGCAGTTCAGAACCACACGGCTACCATGCTGGAGATAGGAACCAGCCT
CCTCTCTCAGACTGCAGAGCAGACCAGAAAGCTGACAGATGTTGAGAC
CCAGGTACTAAATCAAACTTCTCGACTTGAGATACAGCTGCTGGAGAA
TTCATTATCCACCTACAAGCTAGAGAAGCAACTTCTTCAACAGACAAAT
GAAATCTTGAAGATCCATGAAAAAAACAGTTTATTAGAACATAAAATC
TTAGAAATGGAAGGAAAACACAAGGAAGAGTTGGACACCTTAAAGGA
AGAGAAAGAGAACCTTCAAGGCTTGGTTACTCGTCAAACATATATAAT
CCAGGAGCTGGAAAAGCAATTAAACAGAGCTACCACCAACAACAGTGT
CCTTCAGAAGCAGCAACTGGAGCTGATGGACACAGTCCACAACCTTGT
CAATCTTTGCACTAAAGAAGGTGTTTTACTAAAGGGAGGAAAAAGAGA
GGAAGAGAAACCATTTAGAGACTGTGCAGATGTATATCAAGCTGGTTT
TAATAAAAGTGGAATCTACACTATTTATATTAATAATATGCCAGAACCC
AAAAAGGTGTTTTGCAATATGGATGTCAATGGGGGAGGTTGGACTGTA
ATACAACATCGTGAAGATGGAAGTCTAGATTTCCAAAGAGGCTGGAAG
GAATATAAAATGGGTTTTGGAAATCCCTCCGGTGAATATTGGCTGGGG
AATGAGTTTATTTTTGCCATTACCAGTCAGAGGCAGTACATGCTAAGAA
TTGAGTTAATGGACTGGGAAGGGAACCGAGCCTATTCACAGTATGACA
GATTCCACATAGGAAATGAAAAGCAAAACTATAGGTTGTATTTAAAAG
GTCACACTGGGACAGCAGGAAAACAGAGCAGCCTGATCTTACACGGTG
CTGATTTCAGCACTAAAGATGCTGATAATGACAACTGTATGTGCAAATG
TGCCCTCATGTTAACAGGAGGATGGTGGTTTGATGCTTGTGGCCCCTCC
AATCTAAATGGAATGTTCTATACTGCGGGACAAAACCATGGAAAACTG
AATGGGATAAAGTGGCACTACTTCAAAGGGCCCAGTTACTCCTTACGTT
CCACAACTATGATGATTCGACCTTTAGATTTTTGAAAGCGCAATGTCAG
AAGCGATTATGAAAGCAACAAAGAAATCCGGAGAAGCTGCCAGGTGA
GAAACTGTTTGAAAACTTCAGAAGCAAACAATATTGTCTCCCTTCCAGC
AATAAGTGGTAGTTATGTGAAGTCACCAAGGTTCTTGACCGTGAATCTG
GAGCCGTTTGAGTTCACAAGAGTCTCTACTTGGGGTGACAGTGCTCACG
TGGCTCGACTATAGAAAACTCCACTGACTGTCGGGCTTTAAAAAGGGA
AGAAACTGCTGAGCTTGCTGTGCTTCAAACTACTACTGGACCTTATTTT
GGAACTATGGTAGCCAGATGATAAATATGGTTAATTTCATGTAAAACA
GAAAAAAAGAGTGAAAAAGAGAATATACATGAAGAATAGAAACAAGC
CTGCCATAATCCTTTGGAAAAGATGTATTATACCAGTGAAAAGGTGTTA
TATCTATGCAAACCTACTAACAAATTATACTGTTGCACAATTTTGATAA
AAATTTAGAACAGCATTGTCCTCTGAGTTGGTTAAATGTTAATGGATTT
CAGAAGCCTAATTCCAGTATCATACTTACTAGTTGATTTCTGCTTACCC
ATCTTCAAATGAAAATTCCATTTTTGTAAGCCATAATGAACTGTAGTAC
ATGGACAATAAGTGTGTGGTAGAAACAAACTCCATTACTCTGATTTTTG
ATACAGTTTTCAGAAAAAGAAATGAACATAATCAAGTAAGGATGTATG
TGGTGAAAACTTACCACCCCCATACTATGGTTTTCATTTACTCTAAAAA
CTGATTGAATGATATATAAATATATTTATAGCCTGAGTAAAGTTAAAAG
AATGTAAAATATATCATCAAGTTCTTAAAATAATATACATGCATTTAAT
ATTTCCTTTGATATTATACAGGAAAGCAATATTTTGGAGTATGTTAAGT
TGAAGTAAAAGCAAGTACTCTGGAGCAGTTCATTTTACAGTATCTACTT
GCATGTGTATACATACATGTAACTTCATTATTTTAAAAATATTTTTAGA
ACTCCAATACTCACCCTGTTATGTCTTGCTAATTTAAATTTTGCTAATT
AACTGAAACATGCTTACCAGATTCACACTGTTCCAGTGTCTATAAAAGA
AACACTTTGAAGTCTATAAAAAATAAAATAATTATAAATATCATTGTAC
ATAGCATGTTTATATCTGCAAAAAACCTAATAGCTAATTAATCTGGAAT
ATGCAACATTGTCCTTAATTGATGCAAATAACACAAATGCTCAAAGAAA
TCTACTATATCCCTTAATGAAATACATCATTCTTCATATATTTCTCCTT
CAGTCCATTCCCTTAGGCAATTTTTAATTTTTAAAAATTATTATCAGGG
GAGAAAAATTGGCAAAACTATTATATGTAAGGGAAATATATACAAAAAG
AAAATTAATCATAGTCACCTGACTAAGAAATTCTGACTGCTAGTTGCCA
TAAATAACTCAATGGAAATATTCCTATGGGATAATGTATTTTAAGTGAA
TTTTTGGGGTGCTTGAAGTTACTGCATTATTTTATCAAGAAGTCTTCTC
TGCCTGTAAGTGTCCAAGGTTATGACAGTAAACAGTTTTTATTAAAACA
TGAGTCACTATGGGATGAGAAAATTGAAATAAAGCTACTGGGCCTCCTC
TCATAAAAGAGACAGTTGTTGGCAAGGTAGCAATACCAGTTTCAAACT
TGGTGACTTGATCCACTATGCCTTAATGGTTTCCTCCATTTGAGAAAAT
AAAGCTATTCACATTGTTAAGAAAAATACTTTTTAAAGTTTACCATCAA
GTCTTTTTTATATTTATGTGTCTGTATTCTACCCCTTTTTGCCTTACAA
GTGATATTTGCAGGTATTATACCATTTTTCTATTCTTGGTGGCTTCTTC
ATAGCAGGTAAGCCTCTCCTTCTAAAAACTTCTCAACTGTTTTCATTTA
AGGGAAAGAAAATGAGTATTTTGTCCTTTTGTGTTCCTACAGACACTTT
CTTAAACCAGTTTTTGGATAAAGAATACTATTTCCAAACTCATATTACA
AAAACAAAATAAAATAATAAAAAAAGAAAGCATGATATTTACTGTTTTG
TTGTCTGGGTTTGAGAAATGAAATATTGTTTCCAATTATTTATAATAAA
TCAGTATAAAATGTTTTATGATTGTTATGTGTATTATGTAATACGTACA
TGTTTATGGCAATTTAACATGTGTATTCTTTTAATTGTTTCAGAATAGG
ATAATTAGGTATTCGAATTTTGTCTTTAAAATTCATGTGGTTTCTATGC
AAAGTTCTTCATATCATCACAACATTATTTGATTTAAATAAAATTGAAA
GTAATATTTGTGCAA
Gene: Angptl (Ang-1)
Species: mouse
NCBI Accession No.: NM_009640
SEQ ID NO: 652
Sequence:
GGAAAGGGGCTAGAATATGTACTCGCAGCTGACGCGGGCAGGCTCCAC
GCTGAACGGTTACACAGAGAGGAAACAATAAATCTAAGCTACTATTGC
AATAAATATCTCAAGTTTTAACGAAGGAAACTATCATTACAGTTAAAAT
TTTTTAAAGTAACGCTTTTTTAGAACAAAGCTAACAAATGGCTAGTTTT
CTGTGGATCTTCTTCAAACGCTTTCTTTAACGGGGAAAGAGTCAAACAA
GCAGTTTTACCTGAAATAAAGAACTAGTTTAAAGGTCAGAAGAGAAGA
GCAAGCTTTGCAGGAGGCACGGAAGGCAAGCGCTGGCAGTACAATGAC
AGTTTTCCTTTCCTTTGCATTCTTCGCTGCCATTCTGACTCACATAGGG
TGCAGCAACCAGCGCCGAAATCCAGAAAACGGAGGGAGAAGATATAAC
CGGATTCAACATGGGCAATGTGCCTACACTTTCATTCTTCCAGAACACG
ACGGGAACTGCCGTGAGAGTGCGACAGAGCAGTACAACACCAACGCTC
TGCAAAGGGATGCTCCACACGTGGAGCCGGATTTCTCTTCCCAGAAACT
TCAGCATCTGGAGCATGTGATGGAAAATTATACTCAGTGGCTGCAAAA
ACTTGAGAATTACATTGTGGAAAATATGAAGTCGGAGATGGCCCAGAT
ACAACAGAATGCTGTTCAAAACCACACGGCCACCATGCTTGAGATAGG
AACCAGTCTCTTATCTCAGACTGCAGAGCAGACCCGAAAGCTGACAGA
TGTTGAGACCCAGGTACTAAATCAAACATCCCGACTTGAAATACAACT
GCTAGAGAATTCATTATCAACATACAAGCTAGAGAAGCAACTTCTCCA
ACAGACAAATGAAATTCTGAAGATTCACGAAAAAAACAGTTTACTAGA
GCACAAAATCTTAGAAATGGAGGGAAAACACAAAGAAGAATTGGACA
CCTTGAAGGAGGAGAAAGAAAACCTTCAAGGCTTGGTTTCTCGTCAGA
CATTCATCATCCAGGAGTTGGAGAAGCAACTTAGTAGAGCTACCAACA
ACAACAGCATCCTGCAGAAGCAACAACTGGAGCTCATGGACACAGTTC
ATAACCTTATCAGCCTTTGCACTAAAGAAGGTGTTTTGCTAAAGGGAGG
AAAAAGAGAAGAAGAGAAACCATTTCGAGACTGTGCAGATGTATATCA
AGCTGGTTTTAATAAAAGTGGAATCTACACTATTTATTTTAATAATATG
CCAGAACCCAAAAAGGTATTTTGCAATATGGATGTGAATGGGGGAGGT
TGGACAGTAATACAACACCGGGAAGATGGAAGCCTGGATTTCCAGAGG
GGCTGGAAGGAGTATAAAATGGGTTTTGGGAATCCCTCTGGTGAATAT
TGGCTTGGGAACGAGTTCATTTTTGCAATAACCAGTCAGAGGCAGTAC
ATGCTGAGGATTGAGCTGATGGACTGGGAAGGGAACCGAGCCTACTCA
CAGTACGACAGATTCCACATAGGAAATGAAAAGCAGAACTATAGGTTA
TATTTAAAAGGTCACACAGGGACAGCAGGCAAACAGAGCAGCTTGATC
TTACACGGTGCCGATTTCAGCACGAAGGATGCTGATAACGACAACTGT
ATGTGCAAATGCGCTCTCATGCTAACAGGAGGTTGGTGGTTCGATGCCT
GTGGCCCTTCCAATCTAAATGGAATGTTCTACACTGCGGGACAAAATCA
TGGAAAACTGAATGGGATAAAGTGGCACTACTTCAAAGGGCCCAGTTA
CTCCTTACGTTCCACCACCATGATGATCCGGCCCTTGGACTTTTGAAGG
TGCTCTGCCAGTATTAGAAAGCTGCAAAGAAAGCTGGGCATGTTCCCA
GATGAGAAGCTAGTCAGAGGCTTCAGAAACAACCAACATTGTCTCCAT
TCCAGCAGCAAGTGGTTATGTCATGTCACCTGGGTTTGGAGCCTTCTGA
GGTCAACAGAATCGCCACTTGGGTCCAGAGAATGCCACTCACAATCAT
GTTTAAAAGGGAAGAAACTTCTCAGCTTGCTGCACTTCAAAGTGCTACT
GGATCACATTCTGAACTTATAACATCCTGATGCTGAATGCAACTTGTTT
CATGTAAAAGCAAAAGAAGAAGAAACAGCAAATGGGAACAGGCTTTC
CAGAATCTGTTGAAGATGGATTGTGGAGGTGACCTGGTATCACTGTAG
GAAATCCTGCTAACAATACATCACTGCCCAAAAGAGACATAAAGAAAA
GTTTTGTCTACTGAGTTGGCTAAAAGTTAGTGGAGTTCACCTGCCCATT
TCCAGTATCATATTTACTAGCTGATTTCAGGTTTCCTGTGTTCAAATGT
AAACTCTGTTCTTGTAAGCCATGATACAATATAGTACATGGAGGATAAG
AGTTGGGGGTAGAAGGTGCCTAAAGACTCTTGAGTTTCTGGGGATTCA
GTTTTCAAAAGATATAAAATATAATCAAGAATGGATAAAACAGGTGAA
AATCACACTCATGCTACAGTGTTC CTTTACATGAAATTTGATTAACTG
ATCCACAAGAATGTTTAGAGCCTGAGTATATATAAAGACTGGAAGTGTT
ATCACCCAGTTCTCAAAACAATAAGCAGGCAGTTAACATTCTCATTGAC
AGTATGTAGGAGAGCAATATGTGGAGTACTTGAGTTGGAACAGCCCAT
TGTACAGATCTTGCATGTATTTGCATATGTATGGCATTATTATTTTTAA
AGTGTTCGTAGGCCTTCAATTCTTCATACAGATTTTTCATGCTAATTTA
ATTTTTGTTAATTAACTGCAATGTACTTACTAAATATATCCTACTCCAG
TTTTTTATGAGTTATACTTTAAAGTCTACAAATAATAGAAGAATTTTAA
ATATCATTGTACATAATATCTTATACCTGTCCATGCTAAACTCAATAAT
TGTTTAGTCTGGAATATATGATGCTGTCCACAACTGATGACTATAAATA
TGATTGTTTAAAGACAGTTACCATACTATTGATTAAATATATTACTCTG
CATAGTTTTTCTCCTCCAGGATCTGTTTCTTCAAGCAATTTCTACCTTG
TAAAATAATGGTAGTAGAGAAAATTGACATAACTCCTTGTACAAAAGAA
TTATAGAAAAAATTACAGTCATTTGACTAGGAAGTTTCTGATTGTTAGC
TGCTATAAGTGCCTTAGTTAAGATGCCCCTGTGTTATAATATGTAGTAA
ATGAAGTTTTGGACACAGGATTCTGTGATAACCTGATGTGACTGCAGTA
TTCTATCAAGTTCTCTTTGTTGTTAAATGTTCAAGGTTATAGTAGAAAA
AAAACATTCAATCAAACACAATTTGCCATGAAAGGAGAGAACTAAATGT
AGGCACCAGTTCTGTTTTCTCAGAGAAGGAGAAGACTTTCTGGGACGTA
CATGTACCAAAATATAAATCTTGATAACCGCAGCCACAAAGCCTTAGTG
ACTTTCCTCTACCTGGTAAGACAGAGCTCTTCATGCTTTTAAGAAAAGA
TTCTGAATGCTTCCCACCACATCTTTCTTATATTTATATGTGTTCATAA
AGTACTATTTTGCCTTACAAGAGGTATGTGCCGACATTACAGGATTTTT
CTACTATAGTGACTCCTTCACAGCTTTCTTAAGCCTAGCCCTCTAAAAG
CTTCCTTCTCATTTAGATGAAAGAAAATGAGTATTTTTGTGATTCTGGT
GATTGTGGTGGTTGTTGTTGTTGTTGTTGTTGTTCCCACAGATGTTCGA
AAACTCATCTTGGGTAAATTGTTTTTCAATCCACATTACAAAAATAAAG
CGAAACAAGGAGAAAAAAAAGCATGGAATTTACTGATTTGTTATGTGGG
TTTGAAAAATAAGATATTGTTTTCAGTTATTTATAATAAAGCAGTATAA
TGTGTACATTGTATAATGCCAACATGTGTGTAGCAATTTGATACGCATA
GCTTTTTGCATTTAATTAATGCAGGGCAGAAAAATTAGATAACTCGAAC
TTTGTCTTGAAGTTTCTATTTCAATAAAAGCTGTGTCATTTCTATGAAA
A
Gene: ANGPT2 (Ang-2)
Species: mouse
NCBI Accession No.: NM_009640
SEQ ID NO: 653
Sequence:
AAAGTGATTGATTCGGATACTGACACTGTAGGATCTGGGGAGAGAGGA
ACAAAGGACCGTGAAAGCTGCTCTGTAAAAGCTGACACAGCCCTCCCA
AGTGAGCAGGACTGTTCTTCCCACTGCAATCTGACAGTTTACTGCATGC
CTGGAGAGAACACAGCAGTAAAAACCAGGTTTGCTACTGGAAAAAGA
GGAAAGAGAAGACTTTCATTGACGGACCCAGCCATGGCAGCGTAGCAG
CCCTGCGTTTTAGACGGCAGCAGCTCGGGACTCTGGACGTGTGTTTGCC
CTCAAGTTTGCTAAGCTGCTGGTTTATTACTGAAGAAAGAATGTGGCAG
ATTGTTTTCTTTACTCTGAGCTGTGATCTTGTCTTGGCCGCAGCCTATA
ACAACTTTCGGAAGAGCATGGACAGCATAGGAAAGAAGCAATATCAGGT
CCAGCATGGGTCCTGCAGCTACACTTTCCTCCTGCCAGAGATGGACAAC
TGCCGCTCTTCCTCCAGCCCCTACGTGTCCAATGCTGTGCAGAGGGACG
CGCCGCTCGAATACGATGACTCGGTGCAGAGGCTGCAAGTGCTGGAGA
ACATCATGGAAAACAACACTCAGTGGCTAATGAAGCTTGAGAATTATA
TCCAGGACAACATGAAGAAAGAAATGGTAGAGATACAGCAGAATGCA
GTACAGAACCAGACGGCTGTGATGATAGAAATAGGGACAAACCTGTTG
AACCAAACAGCGGAGCAAACGCGGAAGTTAACTGATGTGGAAGCCCA
AGTATTAAATCAGACCACGAGACTTGAACTTCAGCTCTTGGAACACTCC
CTCTCGACAAACAAATTGGAAAAACAGATTTTGGACCAGACCAGTGAA
ATAAACAAATTGCAAGATAAGAACAGTTTCCTAGAAAAGAAGGTGCTA
GCTATGGAAGACAAGCACATCATCCAACTACAGTCAATAAAAGAAGAG
AAAGATCAGCTACAGGTGTTAGTATCCAAGCAAAATTCCATCATTGAA
GAACTAGAAAAAAAAATAGTGACTGCCACGGTGAATAATTCAGTTCTT
CAGAAGCAGCAACATGATCTCATGGAGACAGTTAATAACTTACTGACT
ATGATGTCCACATCAAACTCAGCTAAGGACCCCACTGTTGCTAAAGAA
GAACAAATCAGCTTCAGAGACTGTGCTGAAGTATTCAAATCAGGACAC
ACCACGAATGGCATCTACACGTTAACATTCCCTAATTCTACAGAAGAG
ATCAAGGCCTACTGTGACATGGAAGCTGGAGGAGGCGGGTGGACAATT
ATTCAGCGACGTGAGGATGGCAGCGTTGATTTTCAGAGGACTTGGAAA
GAATATAAAGTGGGATTTGGTAACCCTTCAGGAGAATATTGGCTGGGA
AATGAGTTTGTTTCGCAACTGACTAATCAGCAACGCTATGTGCTTAAAA
TACACCTTAAAGACTGGGAAGGGAATGAGGCTTACTCATTGTATGAAC
ATTTCTATCTCTCAAGTGAAGAACTCAATTATAGGATTCACCTTAAAGG
ACTTACAGGGACAGCCGGCAAAATAAGCAGCATCAGCCAACCAGGAA
ATGATTTTAGCACAAAGGATGGAGACAACGACAAATGTATTTGCAAAT
GTTCACAAATGCTAACAGGAGGCTGGTGGTTTGATGCATGTGGTCCTTC
CAACTTGAACGGAATGTACTATCCACAGAGGCAGAACACAAATAAGTT
CAACGGCATTAAATGGTACTACTGGAAAGGCTCAGGCTATTCGCTCAA
GGCCACAACCATGATGATCCGACCAGCAGATTTCTAAACATCCCAGTC
CACCTGAGGAACTGTCTCGAACTATTTTCAAAGACTTAAGCCCAGTGCA
CTGAAAGTCACGGCTGCGCACTGTGTCCTCTTCCACCACAGAGGGCGTG
TGCTCGGTGCTGACGGGACCCACATGCTCCAGATTAGAGCCTGTAAACT
TTATCACTTAAACTTGCATCACTTAACGGACCAAAGCAAGACCCTAAAC
ATCCATAATTGTGATTAGACAGAACACCTATGCAAAGATGAACCCGAG
GCTGAGAATCAGACTGACAGTTTACAGACGCTGCTGTCACAACCAAGA
ATGTTATGTGCAAGTTTATCAGTAAATAACTGGAAAACAGAACACTTAT
GTTATACAATACAGATCATCTTGGAACTGCATTCTTCTGAGCACTGTTT
ATACACTGTGTAAATACCCATATGTCCTGAATTCACCATCACTATCACA
ATTAAAAGGAAGAAAAAAACTCTCTAAGCCATAAAAAGACATATTCAG
GGATATTCTGAGAAGGGGTTACTAGAAGTTTAATATTTGGAAAAACAG
TTAGTGCATTTTTACTCCATCTCTTAGGTGCTTTAAATTTTTATTTCAA
AAACAGCGTATTTACATTTATGTTGACAGCTTAGTTATAAGTTAATGCT
CAAATACGTATTTCAAATTTATATGGTAGAAACTTCCAGAATCTCTGAA
ATTATCAACAGAAACGTGCCATTTTAGTTTATATGCAGACCGTACTATT
TTTTTCTGCCTGATTGTTAAATATGAAGGTATTTTTAGTAATTAAATAT
AACTTATTAGGGGATATGCCTATGTTTAACTTTTATGATAATATTTACA
ATTTTATAATTTGTTTCCAAAAGACCTAATTGTGCCTTGTGATAAGGAA
ACTTCTTACTTTTAATGATGAGGAAAATTATACATTTCATTCTATGACA
AAGAAACTTTACTATCTTCTCACTATTCTAAAACAGAGGTCTGTTTTCT
TTCCTAGTAAGATATATTTTTATAGAACTAGACTACAATTTAATTTCTG
GTTGAGAAAAGCCTTCTATTTAAGAAATTTACAAAGCTATATGTCTCAA
GATTCACCCTTAAATTTACTTAAGGAAAAAAATAATTGACACTAGTAAG
TTTTTTTATGTCAATCAGCAAACTGAAAAAAAAAAAAGGGTTTCAAAGT
GCAAAAACAAAATCTGATGTTCATAATATATTTAAATATTTACCAAAAA
TTTGAGAACACAGGGCTGGGCGCAGTGGCTCACACCTATAATCCCAGTA
CATTGGTAGGCAAGGTGGGCAGATCACCTGAGGTCAGGAGTTCAAGACC
AGCCTGGACAACATGGTGAAACCCTGTCTCTACTAAATAATACAAAAAT
TAGCCAGGCGTGCTGGCGGGCACCTGTAATCCCAGCTACTCGGGAGGC
TGAGGCAGGGAGAATTGCTTGCACCAGGGAGGTAGAGGTTGCAGTGAG
CCAAGATCGCACCACTGCACTCCAGCCGGGGCAACAGAGCAAGACTCC
ATCTCAAAAAAAAAAAAAAAAAAAGAAAGAAAAGAAAATTTGAGAAC
ACAGCTTTATACTCGGGACTACAAAACCATAAACTCCTGGAGTTTTAAC
TCCTTTTGAAATTTTCATAGTACAATTAATACTAATGAACATTTGTGTA
AAGCTTTATAATTTAAAGGCAATTTCTCATATATTCTTTTCTGAATCAT
TTGCAAGGAAGTTCAGAGTCCAGTCTGTAACTAGCATCTACTATATGTC
TGTCTTCACCTTACAGTGTTCTACCATTATTTTTTCTTTATTCCATTTC
AAAATCTAATTTATTTTACCCCAACTTCTCCCCACCACTTGACGTAGTT
TTAGAACACACAGGTGTTGCTACATATTTGGAGTCAATGATGGACTCTG
GCAAAGTCAAGGCTCTGTTTTATTTCCACCAAGGTGCACTTTTCCAACA
ACTATTTAACTAGTTAAGAACCTCCCTATCTTAGAACTGTATCTACTTT
ATATTTAAGAAGGTTTTATGAATTCAACAACGGTATCATGGCCTTGTAT
CAAGTTGAAAAACAACTGAAAATAAGAAAATTTCACAGCCTCGAAAGAC
AACAACAAGTTTCTAGGATATCTCAATGACAAGAGTGATGGATACTTAG
GTAGGGAAACGCTAATGCAGGAAAAACTGGCAACAACACAATTTATATC
AATTCTCTTTGTAGGCAGGTGATAAAAAATTCAAGGACAAATCTCATTA
TGTCATTGTGCATCATATATAATCTCTTATGAGCGAGAATGGGGGGAAT
TTGTGTTTTTACTTTACACTTCAATTCCTTACACGGTATTTCAAACAAA
CAGTTTTGCTGAGAGGAGCTTTTGTCTCTCCTTAAGAAAATGTTTATAA
AGCTGAAAGGAAATCAAACAGTAATCTTAAAAATGAAAACAAAACAACC
CAACAACCTAGATAACTACAGTGATCAGGGAGCACAGTTCAACTCCTTG
TTATGTTTTAGTCATATGGCCTACTCAAACAGCTAAATAACAACACCAG
TGGCAGATAAAAATCACCATTTATCTTTCAGCTATTAATCTTTTGAATG
AATAAACTGTGACAAACAAATTAACATTTTTGAACATGAAAGGCAACT
TCTGCACAATCCTGTATCCAAGCAAACTTTAAATTATCCACTTAATTAT
TACTTAATCTTAAAAAAAATTAGAACCCAGAACTTTTCAATGAAGCATT
TGAAAGTTGAAGTGGAATTTAGGAAAGCCATAAAAATATAAATACTGT
TATCACAGCACCAGCAAGCCATAATCTTTATACCTATCAGTTCTATTTC
TATTAACAGTAAAAACATTAAGCAAGATATAAGACTACCTGCCCAAGA
ATTCAGTCTTTTTTCATTTTTGTTTTTCTCAGTTCTGAGGATGTTAATC
GTCAAATTTTCTTTGGACTGCATTCCTCACTACTTTTTGCACAATGGTC
TCACGTTCTCACATTTGTTCTCGCGAATAAATTGATAAAAGGTGTTAAG
TTCTGTGAATGTCTTTTTAATTATGGGCATAATTGTGCTTGACTGGATA
AAAACTTAAGTCCACCCTTATGTTTATAATAATTTCTTGAGAACAGCAA
ACTGCATTTACCATCGTAAAACAACATCTGACTTACGGGAGCTGCAGGG
AAGTGGTGAGACAGTTCGAACGGCTCCTCAGAAATCCAGTGACCCAATT
CTAAAGACCATAGCACCTGCAAGTGACACAACAAGCAGATTTATTATAC
ATTTATTAGCCTTAGCAGGCAATAAACCAAGAATCACTTTGAAGACAC
AGCAAAAAGTGATACACTCCGCAGATCTGAAATAGATGTGTTCTCAGA
CAACAAAGTCCCTTCAGAATCTTCATGTTGCATAAATGTTATGAATATT
AATAAAAAGTTGATTGAGAAAAA
Gene: Angpt2 (Ang-2)
Species: mouse
NCBI Accession No.: NM_007426
SEQ ID NO: 654
Sequence:
GATACTGACACTGTAGACTCAGGGGAGAAACAAAGAGTCCGTGCAGAC
CTCTGGAGTGAGCAGGGCTGCTCCTTCCTCTCAGGACAGCTCCGAGTGT
GCCGGGGAGAAGAGAAGAGAAGAGACAGGCACTGGGAAAGAGCCTGC
TGCGGGACGGAGAAGGCTCTCACTGATGGACTTATTCACACGGCACAG
CCCTGTGCCTTAGACAGCAGCTGAGAGCTCAGGACGCAAGTTTGCTGA
ACTCACAGTTTAGAACCCAAAAAGAGAGAGAGAATGTGGCAGATCATT
TTCCTAACTTTTGGCTGGGATCTTGTCTTGGCCTCAGCCTACAGTAACT
TTAGGAAGAGCGTGGACAGCACAGGCAGAAGGCAGTACCAGGTCCAGA
ACGGACCCTGCAGCTACACGTTCCTGCTGCCGGAGACCGACAGCTGCC
GATCTTCCTCCAGCCCCTACATGTCCAATGCCGTGCAGAGGGATGCACC
CCTCGACTACGACGACTCAGTGCAAAGGCTGCAGGTGCTGGAGAACAT
TCTAGAGAACAACACACAGTGGCTGATGAAGCTGGAGAATTACATTCA
GGACAACATGAAGAAGGAGATGGTGGAGATCCAACAGAATGTGGTGC
AGAACCAGACAGCTGTGATGATAGAGATTGGAACCAGCTTGCTGAACC
AGACAGCAGCACAAACTCGGAAACTGACTGATGTGGAAGCCCAAGTAC
TAAACCAGACGACAAGACTCGAGCTGCAGCTTCTCCAACATTCTATTTC
TACCAACAAATTGGAAAAGCAGATTTTGGATCAGACCAGTGAAATAAA
CAAGCTACAAAATAAGAACAGCTTCCTAGAACAGAAAGTTCTGGACAT
GGAGGGCAAGCACAGCGAGCAGCTACAGTCCATGAAGGAGCAGAAGG
ACGAGCTCCAGGTGCTGGTGTCCAAGCAGAGCTCTGTCATTGACGAGC
TGGAGAAGAAGCTGGTGACAGCCACGGTCAACAACTCGCTCCTTCAGA
AGCAGCAGCATGACCTAATGGAGACCGTCAACAGCTTGCTGACCATGA
TGTCATCACCCAACTCCAAGAGCTCGGTTGCTATCCGTAAAGAAGAGC
AAACCACCTTCAGAGACTGTGCGGAAATCTTCAAGTCAGGACTCACCA
CCAGTGGCATCTACACACTGACCTTCCCCAACTCCACAGAGGAGATCA
AGGCCTACTGTGACATGGACGTGGGTGGAGGAGGGTGGACAGTCATCC
AACACCGAGAAGATGGCAGTGTGGACTTCCAGAGGACGTGGAAAGAA
TACAAAGAGGGCTTCGGGAGCCCTCTGGGAGAGTACTGGCTGGGCAAT
GAGTTTGTCTCCCAGCTGACCGGTCAGCACCGCTACGTGCTTAAGATCC
AGCTGAAGGACTGGGAAGGCAACGAGGCGCATTCGCTGTATGATCACT
TCTACCTCGCTGGTGAAGAGTCCAACTACAGGATTCACCTTACAGGACT
CACGGGGACCGCGGGCAAAATAAGTAGCATCAGCCAACCAGGAAGTG
ATTTTAGCACAAAGGATTCGGACAATGACAAATGCATCTGCAAGTGTT
CCCAGATGCTCTCAGGAGGCTGGTGGTTTGACGCATGTGGTCCTTCCAA
CTTGAATGGACAGTACTACCCACAAAAACAGAATACAAATAAGTTTAA
CGGTATCAAGTGGTACTACTGGAAGGGGTCCGGCTACTCGCTCAAGGC
CACAACCATGATGATCCGGCCAGCAGATTTCTAAATGCCTGCCTACACT
ACCAGAAGAACTTGCTGCATCCAAAGATTAACTCCAAGGCACTGAGAG
ACACCAATGCATAGCAGCCCCTTTCCACATCAGGAAGTGCTCCTGGGG
GTGGGGAGGGTCTGTGTGTACCAGACTGAAGCGCATCACTTAAGCCTG
CACCGCTAACCAACCAAAGGCACTGCAGTCTGGAGAAACACTTCTGGG
AAGGTTGTGGCTGAGGATCAGAAGGACAGCGTGCAGACTCTGTCACAG
GGAAGAATGTTCCGTGGGAGTTCAGCAGTAAATAACTGGAAAACAGAA
CACTTAGATGGTGCAGATAAATCTTGGGACCACATTCCTCTAAGCACGG
TTTCTAGAGTGAATACATTCACAGCTCGGCTGTCACAATGACAAGGCCG
TGTCCTCGCACTGTGGCAGCCAGTATCCAGGGACTTCTAAGTGGTGGGC
ACAGGTTATCATCTGGAGAAGCACACATTCATTGTTTTCCTCTTGGGTG
CTTTACATGTTCATTTGAAAACAACACATTTACCTATCTTGATGGCTTA
GTTTTTAATGGCTGGCTACTATTTACTATATGGCAAAAATGCCCACATC
TCTGGAATAACCACCAAATAAGCGCCATGTTGGTGAATGCGGAGACTG
TACTATTTTGTTTTCTTCCTGGCTGTTAAATATGAAGGTATTTTTAGTA
ATTAAATATAAGTTATT

1. A nucleic acid molecule that reduces expression of an angiopoietin-1 (Ang-1), an angiopoietin-2 (Ang-2), or a tyrosine kinase with immunoglobulin and EGF factor homology domains (Tie2) gene, wherein the nucleic acid molecule comprises or targets any one of SEQ ID NOs: 1-648.

2. A nucleic acid molecule that reduces expression of an Ang-2 gene, wherein the nucleic acid molecule comprises or targets any one of SEQ ID NOs: 487, 489, 525, 526, 553, 554, 639, 640, 643, and 644.

3. The nucleic acid molecule of claim 1, wherein the nucleic acid molecule is a short interfering RNA (siRNA) molecule.

4. The siRNA molecule of claim 3, wherein the siRNA molecule is a 25-basepair blunt-ended siRNA molecule.

5. A composition comprising the nucleic acid molecule of claim 1.

6. The composition of claim 5, further comprising a pharmaceutically acceptable carrier.

7. The composition of claim 5, further comprising a nanoparticle.

8. The composition of claim 7, further comprising a histidine-lysine copolymer.

9. The composition of claim 7, further comprising a targeting moiety.

10. The composition of claim 5, further comprising one or more additional therapeutic agents.

11. The composition of claim 5, further comprising one or more additional nucleic acid molecules that induce RNA interference and decrease the expression of a gene of interest.

12. The composition of claim 11, wherein the one or more additional nucleic acid molecules decrease the expression of Ang-1, Ang-2, or Tie-2.

13. A method for reducing protein level expression of Ang-1, Ang-2, or Tie-2 genes in a cell, comprising introducing into the cell the nucleic acid molecule of claim 1.

14. A method of reducing angiogenesis in a subject in need thereof, comprising administering to the subject the nucleic acid molecule of claim 1.

15. A method of treating cancer in a subject in need thereof, comprising administering to the subject the nucleic acid molecule of claim 1.

16. A method for reducing protein level expression of Ang-1, Ang-2, or Tie-2 genes in a cell, comprising introducing into the cell the nucleic acid molecule of claim 2.

17. A method of reducing angiogenesis in a subject in need thereof, comprising administering to the subject the nucleic acid molecule of claim 2.

18. A method of treating cancer in a subject in need thereof, comprising administering to the subject the nucleic acid molecule of claim 2.

19. The nucleic acid molecule of claim 1, wherein the nucleic acid molecule comprises at least one chemical analogue of a nucleotide.

20. The nucleic acid molecule of claim 2, wherein the nucleic acid molecule comprises at least one chemical analogue of a nucleotide.
